(19) United States
(12) Patent Application Publication
(10) Pub. No.: US 2025/0345524 A1
(43) Pub. Date: Nov. 13, 2025

(54) SYRINGES CONTAINING PHARMACEUTICAL COMPOSITIONS COMPRISING RNA

(71) Applicant: CureVac SE, Tübingen (DE)

(72) Inventors: Alexander SCHWENGER, Tübingen (DE); Stefan ARNOLD, Tübingen (DE); Thomas PRUSS, Tübingen (DE); Stefan HEINZ, Tübingen (DE); Tilmann ROOS, Tübingen (DE); Sandra WEIK, Tübingen (DE); Ronja-Katharina KROONEN, Tübingen (DE)

(73) Assignee: CureVac SE, Tübingen (DE)

(21) Appl. No.: 18/553,531

(22) PCT Filed: Mar. 31, 2022
(86) PCT No.: PCT/EP2022/058690

§ 371 Date: Sep. 29, 2023

(30) Foreign Application Priority Data
Mar. 31, 2021 (WO) PCT/EP2021/058486

(51) Int. Cl. A61M 5/31; A61K 31/713; A61M 5/34

(52) U.S. Cl. CPC A61M 5/3129; A61K 31/713; A61M 5/3148; A61M 5/345; A61M 2005/3114; A61M 2205/0222; A61M 2205/0238

(57) ABSTRACT

The present invention is inter alia directed to syringes suitable for compositions comprising RNA, preferably RNA formulated in lipid-based carriers. In particular, kit or kit of parts comprising said suitable syringes are provided. The invention also relates to pre-filled syringes containing pharmaceutical compositions comprising RNA, preferably RNA formulated in lipid-based carriers. Also provided are methods of treating or preventing disorders or diseases, and first and second medical uses. Further the invention is directed to different analytic methods for determining the suitability of syringes for compositions comprising RNA and for detecting RNA agglomerations. Moreover, uses of suitable syringes e.g. for reducing or preventing RNA agglomeration, are described.

This application is a national phase application under 35 U.S.C. § 371 of International Application No. PCT/EP2022/058690, filed Mar. 31, 2022, the entire contents of which are hereby incorporated by reference. International Application No. PCT/EP2022/058690 claims benefit of International Application No. PCT/EP2021/058486, filed Mar. 31, 2021.

### INTRODUCTION

The present invention is inter alia directed to syringes suitable for compositions comprising RNA, preferably RNA formulated in lipid-based carriers. In particular, kit or kit of parts comprising said suitable syringes are provided. The invention also relates to pre-filled syringes containing pharmaceutical compositions comprising RNA, preferably RNA formulated in lipid-based carriers. Also provided are methods of treating or preventing disorders or diseases, and first and second medical uses. Further the invention is directed to different analytic methods for determining the suitability of syringes for compositions comprising RNA and for detecting RNA agglomerations. Moreover, uses of suitable syringes e.g. for reducing or preventing RNA agglomeration, are described.

Therapeutic RNA molecules represent an emerging class of drugs. RNA-based therapeutics include mRNA molecules encoding antigens for use as vaccines. In addition, it is envisioned to use RNA molecules for replacement therapies, e.g. providing missing proteins such as growth factors or enzymes to patients. Furthermore, the therapeutic use of noncoding immunostimulatory RNA molecules (e.g. WO2009/095226A2) and other noncoding RNAs such as microRNAs and long noncoding RNAs or RNAs suitable for genome editing (e.g. CRISPR/Cas9 guide RNAs) is considered. Accordingly, RNA-based therapeutics with the use in immunotherapy, gene therapy, and vaccination belong to the most promising and quickly developing therapeutics in modern medicine. For being effective, RNA is typically delivered by formulating the RNA in lipid-based carrier systems, e.g. liposomes and lipid nanoparticles (LNPs), or by complexation of the RNA in cationic or polycationic compounds (PEI, cationic or polycationic peptides or proteins, e.g. Protamine).

For administration of RNA, e.g. RNA formulated in lipid-based carriers, injection via syringes is typically used (e.g. intramuscular, intradermal, intravenous, intraocular injection, etc.).

It has been described in the art that syringe lubricants may have an impact on the quality of the medicament the syringes contain. For example U.S. Ser. No. 10/471,212B2 describes syringes and pre-filled syringes that are particularly suitable for insulin, vaccines, antibodies, blood products, hormones, cytokines, and the like. The problems to be solved with U.S. Ser. No. 10/471,212B2 are reducing the contamination, degradation, and protein aggregation in peptide or protein-based medicaments.

In regards of drug safety and stability of RNA medicaments, it is of course desirable that the syringe used for administration does not alter the physiochemical characteristics of the composition comprising RNA, e.g. RNA formulated in lipid-based carriers.

This is even more important in the case of pre-filled syringes where the pharmaceutical compositions are stored in the syringe for a long period of time (e.g. weeks or months). Accordingly, careful selection of suitable syringe may be of important for the safe and effective administration of pharmaceuticals comprising RNA, e.g. RNA delivered by lipid-based carrier systems. However, it is not known in the art whether the type of syringe has any negative effect on a contained RNA-based medicament.

The underlying object is therefore to provide syringes that are suitable for the administration and/or storage of pharmaceutical composition comprising RNA, in particular RNA formulated in lipid-based carriers.

A further object of the invention is to provide solutions for the as yet undescribed problem of RNA agglomerations that can be produced by certain types of syringes.

Accordingly, objects of the invention are inter alia to provide syringes (in form of kits or pre-filled syringes) that do not alter the physio-chemical and functional properties of the RNA (and, optionally the lipid-based carrier) comprised in the pharmaceutical composition and also to provide methods for identifying such suitably syringes, uses of the syringes, and medical applications.

The objects mentioned above are solved by the underlying description and the accompanying claims.

### SHORT DESCRIPTION OF THE INVENTION

The inventors discovered that certain types of syringes are not suitable for the administration and/or storage of pharmaceutical compositions comprising RNA as such syringes produce an agglomeration of the RNA. This finding was unexpected, as a negative impact of certain syringes on RNA has not yet been reported in the art. RNA agglomeration is an unwanted effect on the RNA pharmaceutical and has to be avoided, as side-effects after administration to a subject can not be excluded. In further experiments, the inventors identified certain syringes that are suitable for RNA, in particular syringes that produce less RNA agglomeration. Moreover, the inventors developed test procedures for determining the suitability of syringes for compositions comprising RNA, and for determining RNA agglomeration. These methods can be used as quality controls (see Example section).

In a first aspect, the invention provides a kit or kit of parts comprising (A) a syringe and (B) a pharmaceutical composition comprising RNA, wherein the syringe of component A is characterized by at least one of the following features:
(i) the inner surface of the syringe barrel and/or the syringe plunger stopper of is essentially free of silicone oils;
(ii) the syringe of produces less than about 20% RNA agglomeration when incubated with an aqueous test formulation comprising RNA encapsulated in LNPs for 6 hours at 20° C.;
(iii) the syringe produces less than about 20% RNA agglomeration when incubated with the pharmaceutical composition of component B for 6 hours at 20° C.; or
(iv) the syringe produces less than 10 mAU*min of extractable compounds in 20 μl of a 2-Propanol extract as determined by analytical (RP)HPLC.

In preferred embodiments of the first aspect, the kit or kit of parts comprising the following components
(A) a syringe for injection, and (B) a pharmaceutical composition comprising RNA, wherein the syringe of component A is characterized by that (i) the inner surface of the syringe barrel and/or the syringe plunger stopper is essentially free of silicone oils, wherein, optionally, the RNA of component B is formulated in lipid-based carriers and/or the RNA of component B is a single stranded RNA.

In a second aspect, the invention provides a pre-filled syringe containing a pharmaceutical composition comprising RNA, preferably wherein less than 20% of the RNA of the pharmaceutical composition is agglomerated in the syringe, wherein the syringe used for obtaining the pre-filled syringe is characterized by at least one of the following features
(i) the inner surface of the syringe barrel and/or the syringe plunger stopper of is essentially free of silicone oils;
(ii) the syringe produces less than about 20% RNA agglomeration when incubated with an aqueous test formulation comprising RNA encapsulated in LNPs for 6 hours at 20° C.;
(iii) the syringe produces less than about 20% RNA agglomeration when incubated with the pharmaceutical composition comprising RNA for 6 hours at 20° C.; or
(iv) the syringe produces less than 10 mAU*min of extractable compounds in 20 μl of a 2-Propanol extract as determined by analytical RP-HPLC.

In preferred embodiments of the second aspect, the pre-filled syringe for injection containing a pharmaceutical composition comprising RNA, preferably wherein less than 20% of the RNA of the contained pharmaceutical composition is agglomerated, wherein the syringe used for obtaining the pre-filled syringe is characterized by that (i) the inner surface of the syringe barrel and/or the syringe plunger stopper is essentially free of silicone oils, wherein, optionally, the RNA of component B is formulated in lipid-based carriers and/or the RNA of component B is a single stranded RNA.

Accordingly, in particularly preferred embodiments, the second aspect relates to a silicone-oil free pre-filled syringe containing a pharmaceutical composition comprising RNA, wherein, optionally, the RNA of component B is formulated in lipid-based carriers and/or the RNA of component B is a single stranded RNA.

In a third aspect, the invention relates to the medical use of the kit or kit of parts as defined in the first aspect, or the pre-filled syringe as defined in the second aspect.

In a fourth aspect, the invention relates to the medical use of the kit or kit of parts as defined in the first aspect, or the pre-filled syringe as defined in the second aspect as a vaccine.

In a further aspect, the invention relates to the medical use of the kit or kit of parts as defined in the first aspect, or the pre-filled syringe as defined in the second aspect for use in the treatment or prophylaxis of a tumour disease, or of a disorder related to such tumour disease or for use in the treatment or prophylaxis of a genetic disorder or condition or for use in the treatment or prophylaxis of a protein or enzyme deficiency or protein replacement or for use in the treatment or prophylaxis of an infection, or of a disorder related to such an infection.

In a fifth aspect, the invention provides a method of treating or preventing a disorder or condition in a subject wherein the method comprises the following steps
(A) obtaining a pre-filled syringe as defined in the second aspect; or
(B) obtaining a kit or kit of parts as defined the first aspect and (B1) preparing a syringe comprising RNA, preferably RNA formulated in lipid-based carriers; and
(C) injecting the pharmaceutical composition comprising RNA into the subject in need thereof using the syringe obtained in A) or B1).

In a sixth aspect, the present invention relates to a method for providing stable storage of a pharmaceutical composition comprising RNA:
a) obtaining a liquid pharmaceutical composition comprising RNA;
b) transferring the liquid composition to a syringe, wherein the syringe is characterized by at least one of the following features
   (i) the inner surface of the syringe barrel and/or the syringe plunger stopper of is essentially free of silicone oils;
   (ii) the syringe produces less than about 20% RNA agglomeration when incubated with an aqueous test formulation comprising RNA encapsulated in LNPs for 6 hours at 20° C.;
   (iii) the syringe produces less than about 20% RNA agglomeration when incubated with the pharmaceutical composition comprising RNA for 6 hours at 20° C.; or
   (v) the syringe produces less than 10 mAU*min of extractable compounds in 20 μl of a 2-Propanol extract as determined by analytical RP-HPLC.

In an seventh aspect, the invention provides a method for determining the suitability of a syringe for storing a pharmaceutical composition comprising RNA, the method comprising the following steps:
A) drawing a solvent into an empty syringe;
B) emptying the syringe to obtain a solvent extract;
C) determining the amount of compounds in the obtained solvent extract produced by the syringe;
D) assigning suitability of the syringe based on the amount of compounds determined in the solvent extract.

In an eighth aspect, the invention provides a method for determining the suitability of a syringe for storing a pharmaceutical composition comprising RNA, the method comprising the following steps:
A) drawing a composition comprising RNA into an empty syringe;
B) incubating the syringe containing the composition;
C) determining the amount of RNA agglomeration in the composition after incubation produced by the syringe; D) assigning suitability of the syringe based on the amount of RNA agglomeration.

In a ninth aspect, the invention provides a method for determining RNA agglomeration upon exposure of a composition comprising RNA with an article, the method comprising the following steps
A) adding a composition comprising RNA into an article;
B) incubating the article comprising the composition;
C) determining the amount of RNA agglomeration in the composition after incubation produced by the article.

In a tenth aspect, the invention provides the use of a syringe for storing a pharmaceutical composition or vaccine comprising RNA, wherein the syringe is characterized by at least one of the following features
(i) the inner surface of the syringe barrel and/or the syringe plunger stopper of is essentially free of silicone oils;
(ii) the syringe produces less than about 20% RNA agglomeration when incubated with an aqueous test formulation comprising RNA encapsulated in LNPs for 6 hours at 20° C.;
(iii) the syringe produces less than about 20% RNA agglomeration when incubated with the pharmaceutical composition comprising RNA for 6 hours at 20° C.; or
(v) the syringe produces less than 10 mAU*min of extractable compounds in 20 μl of a 2-Propanol extract as determined by analytical RP-HPLC.

In a eleventh aspect, the invention provides the use of a syringe for reducing or preventing RNA agglomeration of a pharmaceutical composition or vaccine comprising RNA, wherein the syringe is characterized by at least one of the following features
(i) the inner surface of the syringe barrel and/or the syringe plunger stopper of is essentially free of silicone oils;
(ii) the syringe produces less than about 20% RNA agglomeration when incubated with an aqueous test formulation comprising RNA encapsulated in LNPs for 6 hours at 20° C.;
(iii) the syringe produces less than about 20% RNA agglomeration when incubated with the pharmaceutical composition comprising RNA for 6 hours at 20° C.; or
(v) the syringe produces less than 10 mAU*min of extractable compounds in 20 μl of a 2-Propanol extract as determined by analytical RP-HPLC.

In a twelfth aspect, the invention provides the medical use of a pharmaceutical composition comprising RNA formulated in lipid-based carriers, wherein the pharmaceutical composition is administered to a subject using a syringe for injection that is characterized by that (i) the inner surface of the syringe barrel and/or the syringe plunger stopper is essentially free of silicone oils.

In a thirteenth aspect, the invention provides a method of treating or preventing a disease, disorder or condition, wherein the method comprises applying or administering to a subject in need thereof an effective amount of a pharmaceutical composition comprising RNA formulated in lipid-based carriers, wherein the applying or administering is an injection using a syringe for injection that is characterized by that (i) the inner surface of the syringe barrel and/or the syringe plunger stopper is essentially free of silicone oils

### Definitions

For the sake of clarity and readability the following definitions are provided. Any technical feature mentioned for these definitions may be read on each and every embodiment of the invention. Additional definitions and explanations may be specifically provided in the context of these embodiments.

Percentages in the context of numbers should be understood as relative to the total number of the respective items. In other cases, and unless the context dictates otherwise, percentages should be understood as percentages by weight (wt.-%).

About: The term “about” is used when determinants or values do not need to be identical, i.e. 100% the same. Accordingly, “about” means, that a determinant or values may diverge by 0.1% to 20%, preferably by 0.1% to 10%; in particular, by 0.5%, 1%, 2%, 3%, 4%, 5%, 6%, 7%, 8%, 9%, 10%, 11%, 12%, 13%, 14%, 15%, 16%, 17%, 18%, 19%, 20%. The skilled person knows that e.g. certain parameters or determinants can slightly vary based on the method how the parameter has been determined. For example, if a certain determinants or value is defined herein to have e.g. a length of “about 1000 nucleotides”, the length may diverge by 0.1% to 20%, preferably by 0.1% to 10%; in particular, by 0.5%, 1%, 2%, 3%, 4%, 5%, 6%, 7%, 8%, 9%, 10%, 11%, 12%, 13%, 14%, 15%, 16%, 17%, 18%, 19%, 20%. Accordingly, the skilled person knows that in that specific example, the length may diverge by 1 to 200 nucleotides, preferably by 1 to 200 nucleotides; in particular, by 5, 10, 20, 30, 40, 50, 60, 70, 80, 90, 100, 110, 120, 130, 140, 150, 160, 170, 180, 190, 200 nucleotides.

Adaptive immune response: The term “adaptive immune response” as used herein will be recognized and understood by the person of ordinary skill in the art, and is e.g. intended to refer to an antigen-specific response of the immune system (the adaptive immune system). Antigen specificity allows for the generation of responses that are tailored to specific pathogens or pathogen-infected cells. The ability to mount these tailored responses is usually maintained in the body by “memory cells” (B-cells). In the context of the invention, the antigen is provided by an RNA encoding at least one antigenic peptide or protein derived from a pathogen (e.g. a virus).

Antigen: The term “antigen” as used herein will be recognized and understood by the person of ordinary skill in the art, and is e.g. intended to refer to a substance which may be recognized by the immune system, preferably by the adaptive immune system, and is capable of triggering an antigen-specific immune response, e.g. by formation of antibodies and/or antigen-specific T cells as part of an adaptive immune response. Typically, an antigen may be or may comprise a peptide or protein which may be presented by the MHC to T-cells. Also fragments, variants and derivatives of peptides or proteins comprising at least one epitope are understood as antigens in the context of the invention. In the context of the present invention, an antigen may be the product of translation of a provided RNA as specified herein.

Antigenic peptide or protein: The term “antigenic peptide or protein” or “immunogenic peptide or protein” will be recognized and understood by the person of ordinary skill in the art, and is e.g. intended to refer to a peptide, protein derived from a (antigenic or immunogenic) protein which stimulates the body's adaptive immune system to provide an adaptive immune response. Therefore an antigenic/immunogenic peptide or protein comprises at least one epitope (as defined herein) or antigen (as defined herein) of the protein it is derived from.

Cationic: Unless a different meaning is clear from the specific context, the term “cationic” means that the respective structure bears a positive charge, either permanently or not permanently, but in response to certain conditions such as pH. Thus, the term “cationic” covers both “permanently cationic” and “cationisable”. The term “permanently cationic” means, e.g., that the respective compound, or group, or atom, is positively charged at any pH value or hydrogen ion activity of its environment. Typically, the positive charge results from the presence of a quaternary nitrogen atom. Where a compound carries a plurality of such positive charges, it may be referred to as permanently polycationic.

Cationisable: The term “cationisable” as used herein means that a compound, or group or atom, is positively charged at a lower pH and uncharged at a higher pH of its environment. Also in non-aqueous environments where no pH value can be determined, a cationisable compound, group or atom is positively charged at a high hydrogen ion concentration and uncharged at a low concentration or activity of hydrogen ions. It depends on the individual properties of the cationisable or polycationisable compound, in particular the pKa of the respective cationisable group or atom, at which pH or hydrogen ion concentration it is charged or uncharged. In diluted aqueous environments, the fraction of cationisable compounds, groups or atoms bearing a positive charge may be estimated using the so-called Henderson-Hasselbalch equation which is well-known to a person skilled in the art. E.g., in some embodiments, if a compound or moiety is cationisable, it is preferred that it is positively charged at a pH value of about 1 to 9, preferably 4 to 9, 5 to 8 or even 6 to 8, more preferably of a pH value of or below 9, of or below 8, of or below 7, most preferably at physiological pH values, e.g. about 7.3 to 7.4, i.e. under physiological conditions, particularly under physiological salt conditions of the cell in vivo. In other embodiments, it is preferred that the cationisable compound or moiety is predominantly neutral at physiological pH values, e.g. about 7.0-7.4, but becomes positively charged at lower pH values. In some embodiments, the preferred range of pKa for the cationisable compound or moiety is about 5 to about 7.

Cationic or polycationic compound: The term “cationic or polycationic compound” as used herein will be recognized and understood by the person of ordinary skill in the art, and is for example intended to refer to a charged molecule, which is positively charged at a pH value ranging from about 1 to 9, at a pH value ranging from about 3 to 8, at a pH value ranging from about 4 to 8, at a pH value ranging from about 5 to 8, more preferably at a pH value ranging from about 6 to 8, even more preferably at a pH value ranging from about 7 to 8, most preferably at a physiological pH, e.g. ranging from about 7.2 to about 7.5. Accordingly, a cationic lipid (including lipidoids) may be any positively charged compound or polymer which is positively charged under physiological conditions.

Coding sequence/coding region: The terms “coding sequence” or “coding region” and the corresponding abbreviation “cds” as used herein will be recognized and understood by the person of ordinary skill in the art, and are e.g. intended to refer to a sequence of several nucleotide triplets, which may be translated into a peptide or protein. A coding sequence in the context of the present invention may be an RNA sequence consisting of a number of nucleotides that may be divided by three, which starts with a start codon and which preferably terminates with a stop codon.

Derived from: The term “derived from” as used throughout the present specification in the context of a nucleic acid, i.e. for a nucleic acid “derived from” (another) nucleic acid, means that the nucleic acid, which is derived from (another) nucleic acid, shares e.g. at least 60%, 70%, 80%, 81%, 82%, 83%, 84%, 85%, 86%, 87%, 88%, 89%, 90%, 91%, 92%, 93%, 94%, 95%, 96%, 97%, 98%, or 99% sequence identity with the nucleic acid from which it is derived. The skilled person is aware that sequence identity is typically calculated for the same types of nucleic acids, i.e. for DNA sequences or for RNA sequences. Thus, it is understood, if a DNA is “derived from” an RNA or if an RNA is “derived from” a DNA, in a first step the RNA sequence is converted into the corresponding DNA sequence (in particular by replacing the uracils (U) by thymidines (T) throughout the sequence) or, vice versa, the DNA sequence is converted into the corresponding RNA sequence (in particular by replacing the T by U throughout the sequence). Thereafter, the sequence identity of the DNA sequences or the sequence identity of the RNA sequences is determined. Preferably, a nucleic acid “derived from” a nucleic acid also refers to nucleic acid, which is modified in comparison to the nucleic acid from which it is derived, e.g. in order to increase RNA stability even further and/or to prolong and/or increase protein production. In the context of amino acid sequences (e.g. antigenic peptides or proteins) the term “derived from” means that the amino acid sequence, which is derived from (another) amino acid sequence, shares e.g. at least 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence from which it is derived.

Fragment: The term “fragment” as used throughout the present specification in the context of a nucleic acid sequence (e.g. RNA or a DNA) or an amino acid sequence may typically be a shorter portion of a full-length sequence of e.g. a nucleic acid sequence or an amino acid sequence. Accordingly, a fragment, typically, consists of a sequence that is identical to the corresponding stretch within the full-length sequence. A preferred fragment of a sequence in the context of the present invention, consists of a continuous stretch of entities, such as nucleotides or amino acids corresponding to a continuous stretch of entities in the molecule the fragment is derived from, which represents at least 40%, 50%, 60%, 70%, 80%, 90%, 95% of the total (i.e. full-length) molecule from which the fragment is derived (e.g. a virus protein). The term “fragment” as used throughout the present specification in the context of proteins or peptides may, typically, comprise a sequence of a protein or peptide as defined herein, which is, with regard to its amino acid sequence, N-terminally and/or C-terminally truncated compared to the amino acid sequence of the original protein. Such truncation may thus occur either on the amino acid level or correspondingly on the nucleic acid level. A sequence identity with respect to such a fragment as defined herein may therefore preferably refer to the entire protein or peptide as defined herein or to the entire (coding) nucleic acid molecule of such a protein or peptide. Fragments of proteins or peptides may comprise at least one epitope of those proteins or peptides.

Heterologous: The terms “heterologous” or “heterologous sequence” as used throughout the present specification in the context of a nucleic acid sequence or an amino acid sequence refers to a sequence (e.g. RNA, DNA, amino acid) has to be understood as a sequence that is derived from another gene, another allele, or e.g. another species or virus. Two sequences are typically understood to be “heterologous” if they are not derivable from the same gene or from the same allele. I.e., although heterologous sequences may be derivable from the same organism or virus, in nature, they do not occur in the same nucleic acid or protein.

Humoral immune response: The terms “humoral immunity” or “humoral immune response” will be recognized and understood by the person of ordinary skill in the art, and are e.g. intended to refer to B-cell mediated antibody production and optionally to accessory processes accompanying antibody production. A humoral immune response may be typically characterized, e.g. by Th2 activation and cytokine production, germinal center formation and isotype switching, affinity maturation and memory cell generation. Humoral immunity may also refer to the effector functions of antibodies, which include pathogen and toxin neutralization, classical complement activation, and opsonin promotion of phagocytosis and pathogen elimination.

Identity (of a sequence): The term “identity” as used throughout the present specification in the context of a nucleic acid sequence or an amino acid sequence will be recognized and understood by the person of ordinary skill in the art, and is e.g. intended to refer to the percentage to which two sequences are identical. To determine the percentage to which two sequences are identical, e.g. nucleic acid sequences or amino acid (aa) sequences as defined herein, preferably the aa sequences encoded by the nucleic acid sequence as defined herein or the aa sequences themselves, the sequences can be aligned in order to be subsequently compared to one another. Therefore, e.g. a position of a first sequence may be compared with the corresponding position of the second sequence. If a position in the first sequence is occupied by the same residue as is the case at a position in the second sequence, the two sequences are identical at this position. If this is not the case, the sequences differ at this position. If insertions occur in the second sequence in comparison to the first sequence, gaps can be inserted into the first sequence to allow a further alignment. If deletions occur in the second sequence in comparison to the first sequence, gaps can be inserted into the second sequence to allow a further alignment.

The percentage to which two sequences are identical is then a function of the number of identical positions divided by the total number of positions including those positions which are only occupied in one sequence. The percentage to which two sequences are identical can be determined using an algorithm, e.g. an algorithm integrated in the BLAST program.

Immunogen, immunogenic: The terms “immunogen” or “immunogenic” will be recognized and understood by the person of ordinary skill in the art, and are e.g. intended to refer to a compound that is able to stimulate/induce an immune response. An immunogen in the sense of the present invention is the product of translation of a provided nucleic acid, comprising at least one coding sequence encoding at least one antigenic peptide, protein derived from e.g. a coronavirus protein as defined herein. Typically, an immunogen elicits an adaptive immune response.

Immune response: The term “immune response” will be recognized and understood by the person of ordinary skill in the art, and is e.g. intended to refer to a specific reaction of the adaptive immune system to a particular antigen (so called specific or adaptive immune response) or an unspecific reaction of the innate immune system (so called unspecific or innate immune response), or a combination thereof.

Immune system: The term “immune system” will be recognized and understood by the person of ordinary skill in the art, and is e.g. intended to refer to a system of the organism that protects the organisms from infection. If a pathogen succeeds in passing a physical barrier of an organism and enters this organism, the innate immune system provides an immediate non-specific response. If pathogens evade this innate response, vertebrates possess a second layer of protection, the adaptive immune system. The immune system adapts its response during an infection to improve its recognition of the pathogen. This improved response is then retained after the pathogen has been eliminated, in the form of an immunological memory, and allows the adaptive immune system to mount faster and stronger attacks each time this pathogen is encountered. According to this, the immune system comprises the innate and the adaptive immune system. Each of these two parts typically contains so called humoral and cellular components.

Innate immune system: The term “innate immune system” (also known as non-specific or unspecific immune system) will be recognized and understood by the person of ordinary skill in the art, and is e.g. intended to refer to a system typically comprising the cells and mechanisms that defend the host from infection by other organisms in a non-specific manner. This means that the cells of the innate system may recognize and respond to pathogens in a generic way, but unlike the adaptive immune system, it does not confer long-lasting or protective immunity to the host. The innate immune system may be activated by ligands of pattern recognition receptor e.g. Toll-like, NOD-like, or RIG-1 like receptors etc.

Nucleic acid, nucleic acid molecule: The terms “nucleic acid” or “nucleic acid molecule” as used herein, will be recognized and understood by the person of ordinary skill in the art. The terms “nucleic acid” or “nucleic acid molecule” preferably refers to DNA (molecules) or RNA (molecules). The term is used synonymously with the term polynucleotide. Preferably, a nucleic acid or a nucleic acid molecule is a polymer comprising or consisting of nucleotide monomers that are covalently linked to each other by phosphodiester-bonds of a sugar/phosphate-backbone. The terms “nucleic acid” or “nucleic acid molecule” also encompasses modified nucleic acid (molecules), such as base-modified, sugar-modified or backbone-modified DNA or RNA (molecules) as defined herein.

Nucleic acid sequence. DNA sequence, RNA sequence: The terms “nucleic acid sequence”, “DNA sequence”, “RNA sequence” will be recognized and understood by the person of ordinary skill in the art, and e.g. refer to a particular and individual order of the succession of its nucleotides.

Nucleic acid species. DNA species, RNA species: In the context of the invention, the term “nucleic acid species”, “DNA species”, “RNA species” is not restricted to mean one single nucleic acid, DNA or RNA molecule but is understood to comprise an ensemble of essentially identical nucleic acid, DNA or RNA molecules. Accordingly, it may relate to a plurality of essentially identical nucleic acid molecules, e.g. DNA or RNA molecules.

RNA: The term “RNA” is the usual abbreviation for ribonucleic acid. It is a nucleic acid molecule, i.e. a polymer consisting of nucleotide monomers. These nucleotides are usually adenosine-monophosphate (AMP), uridine-monophosphate (UMP), guanosine-monophosphate (GMP) and cytidine-monophosphate (CMP) monomers or analogs thereof, which are connected to each other along a so-called backbone. The backbone is formed by phosphodiester bonds between the sugar, i.e. ribose, of a first and a phosphate moiety of a second, adjacent monomer. The specific order of the monomers, i.e. the order of the bases linked to the sugar/phosphate-backbone, is called the RNA sequence. RNA can be obtained by transcription of a DNA sequence, e.g., inside a cell. In eukaryotic cells, transcription is typically performed inside the nucleus or the mitochondria. In vivo, transcription of DNA usually results in the so-called premature RNA which has to be processed into so-called messenger-RNA, usually abbreviated as mRNA. Processing of the premature RNA, e.g. in eukaryotic organisms, comprises a variety of different posttranscriptional modifications such as splicing, 5′-capping, polyadenylation, export from the nucleus or the mitochondria and the like. The sum of these processes is also called maturation of RNA. The mature messenger RNA usually provides the nucleotide sequence that may be translated into an amino acid sequence of a particular peptide or protein. Typically, a mature mRNA comprises a 5′-cap, optionally a 5′UTR, a coding sequence, optionally a 3′UTR and a poly(A) sequence. If RNA molecules are of synthetic origin, the RNA molecules are meant not to be produced in vivo, i.e. inside a cell or purified from a cell, but in an in vitro method. An examples for a suitable in vitro method is in vitro transcription.

In addition to messenger RNA, several non-coding types of RNA exist which may be involved in regulation of transcription and/or translation, and immunostimulation and which may also be produced by in vitro transcription.

RNA in vitro transcription: The terms “RNA in vitro transcription” or “in vitro transcription” relate to a process wherein RNA is synthesized in a cell-free system (in vitro). RNA may be obtained by DNA-dependent in vitro transcription of an appropriate DNA template, which is typically a linear DNA template. The promoter for controlling RNA in vitro transcription can be any promoter for any DNA-dependent RNA polymerase. Reagents used in RNA in vitro transcription typically include a DNA template, ribonucleotide triphosphates, a cap analogue, a DNA-dependent RNA polymerase, a ribonuclease (RNase) inhibitor, MgCl₂, a buffer which can also contain antioxidants (e.g. DTT), and/or polyamines such as spermidine at optimal concentrations. After RNA transcription, the DNA template is typically removed using a DNAse digestion step, followed by several purification steps.

T-cell responses: The terms “cellular immunity” or “cellular immune response” or “cellular T-cell responses” as used herein will be recognized and understood by the person of ordinary skill in the art, and are for example intended to refer to the activation of macrophages, natural killer cells (NK), antigen-specific cytotoxic T-lymphocytes, and the release of various cytokines in response to an antigen. In more general terms, cellular immunity is not based on antibodies, but on the activation of cells of the immune system. Typically, a cellular immune response may be characterized e.g. by activating antigen-specific cytotoxic T-lymphocytes that are able to induce apoptosis in cells, e.g. specific immune cells like dendritic cells or other cells, displaying epitopes of foreign antigens on their surface.

Variant (of a sequence): The term “variant” as used throughout the present specification in the context of a nucleic acid sequence will be recognized and understood by the person of ordinary skill in the art, and is e.g. intended to refer to a variant of a nucleic acid sequence derived from another nucleic acid sequence. E.g., a variant of a nucleic acid sequence may exhibit one or more nucleotide deletions, insertions, additions and/or substitutions compared to the nucleic acid sequence from which the variant is derived. A variant of a nucleic acid sequence may at least 50%, 60%, 70%, 80%, 90%, or 95% identical to the nucleic acid sequence the variant is derived from. The variant is a functional variant in the sense that the variant has retained at least 50%, 60%, 70%, 80%, 90%, or 95% or more of the function of the sequence where it is derived from. A “variant” of a nucleic acid sequence may have at least 70%, 75%, 80%, 85%, 90%, 95%, 98% or 99% nucleotide identity over a stretch of at least 10, 20, 30, 50, 75 or 100 nucleotide of such nucleic acid sequence.

The term “variant” as used throughout the present specification in the context of proteins or peptides is e.g. intended to refer to a proteins or peptide variant having an amino acid sequence which differs from the original sequence in one or more mutation(s)/substitution(s), such as one or more substituted, inserted and/or deleted amino acid(s). Preferably, these fragments and/or variants have the same, or a comparable specific antigenic property (immunogenic variants, antigenic variants). Insertions and substitutions are possible, in particular, at those sequence positions which cause no modification to the three-dimensional structure or do not affect the binding region. Modifications to a three-dimensional structure by insertion(s) or deletion(s) can easily be determined e.g. using CD spectra (circular dichroism spectra). A “variant” of a protein or peptide may have at least 70%, 75%, 80%, 85%, 90%, 95%, 98% or 99% amino acid identity over a stretch of at least 10, 20, 30, 50, 75 or 100 amino acids of such protein or peptide. Preferably, a variant of a protein comprises a functional variant of the protein, which means, in the context of the invention, that the variant exerts essentially the same, or at least 40%, 50%, 60%, 70%, 80%, 90% of the immunogenicity as the protein it is derived from.

### DETAILED DESCRIPTION OF THE INVENTION

Where reference is made to “SEQ ID NOs” of other patent applications or patents, said sequences, e.g. amino acid sequences or nucleic acid sequences, are explicitly incorporated herein by reference. For “SEQ ID NOs” provided herein, information under identifier <223> (in the sequence protocol) is also explicitly included herein in its entirety.

### A Kit or Kit of Parts Comprising (A) a Syringe and (B) a Pharmaceutical Composition Comprising RNA

In a first aspect, the present invention provides a kit or kit of parts comprising the following components
A) a syringe, preferably a syringe for injection; and
B) a pharmaceutical composition comprising RNA.

In a preferred embodiment of the invention, the syringe of component A is configured to allow stable storage and stable administration of the pharmaceutical composition comprising RNA. Accordingly, the pharmaceutical composition comprising RNA of component B is stable upon storage or exposure to the syringe of component A.

In embodiments, the pharmaceutical composition comprising RNA of component B is stable after exposure to the syringe of component A, e.g. after storage.

As used herein, “stable” refers to a pharmaceutical composition comprising RNA where the measured values for various physiochemical parameters are within a defined range after storage or exposure to the syringe. In one embodiment, the pharmaceutical composition is analyzed to assess stability according to various parameters. Suitable stability parameters include, without limitation, RNA agglomeration, RNA integrity, Z-average particle size of lipid-based carriers, polydispersity index (PDI), the amount of free RNA in the pharmaceutical composition, encapsulation efficiency of the RNA (proportion of the RNA in percent incorporated in the lipid-based carriers), shape and morphology of the lipid-based carriers, pH, osmolality, and/or turbidity.

Further, “stable” refers to a pharmaceutical composition comprising RNA where the measured values for various functional parameters are within a defined range after storage or exposure to the syringe. In one embodiment, the pharmaceutical composition is analyzed to assess the potency of the liquid composition including for example the expression of the encoded peptide or protein in a cell, the induction of specific antibody titers, the induction of neutralizing antibody titers, the induction of T-cell, the reactogenicity of the liquid composition including for example the induction of innate immune responses etc. In preferred embodiments, the term “stable” as used herein refers to RNA agglomeration or RNA integrity.

Preferably, the term stable in the context of the invention does not refer to the absence or reduction of protein agglomeration or protein aggregation and/or does not refer to protein stability.

As used herein, “storage” may be understood as a prolonged exposure or containment of a composition in an article, e.g. a syringe, wherein the duration of storage may be in a range of about 30 minutes to about 6 months. For example, storage may be for at least about 30 minutes, 1 hour, 2 hours, 6 hours, 1 days, 2 days, 1 week, 2 weeks, 3 weeks, 1 month, 2 months, 3 months, 4 months, 5 months, or 6 months. The temperature conditions under which storage according to the invention may occur can range from −80° C. to about 30° C., preferably in a range from about −20° C. to about 30° C., more preferably in a range from about 5° C. to about 20° C. Preferred examples of a storage temperature in the context of the invention are −80° C., −20° C., 5° C., or room temperature (about 20° C.). Suitably, when reference is made to “storage”, such a storage has to be understood as having a duration of about 30 minutes to about 6 months, and as having a temperature in a range from about 5° C. to about 20° C.

According to preferred embodiments, the kit or kit of parts comprises the following components
A) a syringe, preferably a syringe for injection (herein also referred to as “component A”), and
B) a pharmaceutical composition comprising RNA (herein also referred to as “component B”),
   wherein the syringe of component A is characterized by at least one of the following features i) to iv):
(i) the inner surface of the syringe barrel and/or the syringe plunger stopper is essentially free of silicone oils;
(ii) the syringe produces less than about 20% RNA agglomeration when incubated with an aqueous test formulation comprising RNA encapsulated in LNPs for 6 hours at 20° C.;
(iii) the syringe produces less than about 20% RNA agglomeration when incubated with the pharmaceutical composition of component B for 6 hours at 20° C.; or
(iv) the syringe produces less than 10 mAU*min of extractable compounds in 20 μl of a 2-Propanol extract as determined by analytical (RP)HPLC.

In particularly preferred embodiments, the syringe of component A is characterized by features (i), (ii), (iii), and (iv).

In particularly preferred embodiments, the syringe of component A is characterized by features (i), (ii), and (iii).

In particularly preferred embodiments, the syringe of component A is characterized by features (i), (ii), and (iv).

In particularly preferred embodiments, the syringe of component A is characterized by features (i), (iii), and (iv).

In particularly preferred embodiments, the syringe of component A is characterized by features (i), and (ii).

In particularly preferred embodiments, the syringe of component A is characterized by features (i), and (iii).

In particularly preferred embodiments, the syringe of component A is characterized by features (i), and (iv).

In preferred embodiments, the syringe of component A is characterized by features (ii), (iii), and (iv).

In preferred embodiments, the syringe of component A is characterized by features (ii), (iii).

In preferred embodiments, the syringe of component A is characterized by features (ii), (iv).

In preferred embodiments, the syringe of component A is characterized by features (iii), and (iv).

According to preferred embodiments, the kit or kit of parts comprises the following components
A) a syringe, preferably a syringe for injection (herein also referred to as “component A”), and
B) a pharmaceutical composition comprising RNA (herein also referred to as “component B”), wherein the syringe of component A is characterized by that (i) the inner surface of the syringe barrel and/or the syringe plunger stopper is essentially free of silicone oils.

Preferably, the RNA of component B is formulated in lipid-based carriers (as further specified below). Preferably, the RNA of component B is a single stranded RNA. Preferably, the RNA of component B is not an antisense RNA or an siRNA.

Preferably, component B does not comprise a protein based or peptide based medicament. Preferably, the RNA of component B is a long chain RNA, suitably wherein the long-chain RNA is larger than about 100 nucleotides (e.g. about 100 to about 10,000 nucleotides).

In preferred embodiments, the RNA of component B is a single stranded or long chain RNA formulated in lipid-based carriers (as further specified below).

In preferred embodiments, the RNA of component B is a single stranded or long chain RNA formulated in lipid-based carriers (as further specified below) and component B does not comprise a peptide or protein.

In preferred embodiments, the syringe of component A is characterized by that (i) the inner surface of the syringe barrel and/or the syringe plunger stopper is essentially free of silicone oils and component A is further characterized by at least one of the

following features
(ii) the syringe produces less than about 20% RNA agglomeration when incubated with an aqueous test formulation comprising RNA encapsulated in LNPs for 6 hours at 20° C.;
(iii) the syringe produces less than about 20% RNA agglomeration when incubated with the pharmaceutical composition of component B for 6 hours at 20° C.;
(iv) the syringe produces less than 10 mAU*min of extractable compounds in 20 μl of a 2-Propanol extract as determined by analytical (RP)HPLC; or
(v) a combination of any of (ii) to (iv),
   preferably, wherein the RNA of component B is formulated in lipid-based carriers and/or wherein the RNA of component B is a single stranded or long chain RNA.

Typically, a syringe, in particular a syringe for injection comprises, besides other optional elements, a syringe barrel, a syringe plunger including a stopper, and a needle for injection. A syringe for injection may additionally comprise a needle adapter, a needle hub, and other syringe elements. Typically, some elements of a syringe are configured to reduce the gliding force needed for an injection (e.g. an intramuscular injection) using the syringe. Lubricants or surface coatings can be used to improve the gliding of syringe elements. Often, the surface of a syringe barrel and/or the surface of a syringe plunger stopper are treated with lubricants or surface coatings to reduce the gliding force needed for an injection.

Without whishing to be bound to theory, the presence of silicone oils in syringes for injection (e.g. on the surface of the syringe barrel and/or the plunger stopper) can produce unwanted RNA agglomeration.

Accordingly, the syringe for injection is configured so that the materials of the syringe that are in contact with the pharmaceutical composition comprising RNA, preferably RNA formulated in lipid-based carriers, only comprise a low amount of silicone oils. In that context, a low amount of silicone oil has to be understood as less than about 100 μg/mm² silicone oil. For example, it is preferred that the syringe barrel and/or the syringe plunger stopper comprises less than than about 100 μg/mm² silicone oil, preferably less than about 50 μg/mm² silicone oil.

Suitably, the syringe for injection is configured so that the materials of the syringe that are in contact with the pharmaceutical composition comprising RNA, preferably RNA formulated in lipid-based carriers, are essentially free of silicone oils. Essentially free in the context of the invention relates to an amount of silicone oil of less than about 25 μg/mm², less than about 20 μg/mm², less than about 15 μg/mm², less than about 10 μg/mm², or less than about 5 μg/mm² silicone oil. For example, it is preferred that the syringe barrel and/or the syringe plunger stopper comprises less than about 25 μg/mm² silicone oil, preferably less than about 20 μg/mm², less than about 15 μg/mm², less than about 10 μg/mm², or less than 5 μg/mm².

In preferred embodiments, the inner surface of the syringe barrel is essentially free of silicone oils, the syringe plunger is essentially free of silicone oils, the syringe plunger stopper is essentially free of silicone oils, the needle adapter is essentially free of silicone oils, the needle hub is essentially free of silicone oils, and/or the needle is essentially free of silicone oils.

In particularly preferred embodiments of the invention, the syringe for injection comprises a syringe barrel that has an inner surface that is essentially free of silicone oils and/or a syringe plunger stopper that is essentially free of silicone oils.

In preferred embodiments, the syringe for injection is essentially latex-free, essentially pyrogen-free, essentially PVC-free and/or essentially DEHP-free.

In preferred embodiments, the syringe barrel, the syringe plunger, and/or the syringe plunger stopper comprises a material that is suitable for use in a syringe (system) essentially without silicone oil or with a low amount of silicone oil.

In preferred embodiments, the syringe barrel comprises a polymer, preferably an organic polymer.

Suitably, the polymer is selected from olefin polymer, cyclic olefin copolymer (COP), polypropylene, polysterene, polyethylene and/or polycarbonate. In preferred embodiments, the syringe barrel comprises organic polymer selected from polypropylene, polyethylene and/or polycarbonate. In particularly preferred embodiments, the syringe barrel comprises polypropylene. In particularly preferred embodiments, the syringe barrel comprises cyclic olefin copolymer (COP).

Preferably, the syringe barrel is an essentially silicone-oil free syringe barrel and, accordingly, does not comprise a coating with silicone oil of the inner surface of the syringe barrel.

In preferred embodiments, the syringe barrel comprises glass or a glass coating of the inner surface. Further, the syringe barrel may comprise a silicon dioxide coating.

In preferred embodiments, the syringe plunger comprises a material that is suitable for use in a syringe essentially without silicone oil or with a low amount of silicone oil.

In preferred embodiments, the syringe plunger comprises a polymer, preferably an organic polymer.

Suitably, the polymer of the syringe plunger is selected from olefin polymer, cyclic olefin copolymer, polypropylene, polysterene, polyethylene and/or polycarbonate. In preferred embodiments, the syringe plunger comprises organic polymer selected from polypropylene, polyethylene and/or polycarbonate. Preferably, the syringe plunger is a silicone-oil free syringe plunger and, accordingly, does not comprise a coating with silicone oil.

In preferred embodiments, the syringe plunger stopper comprises a material that is suitable for use in a syringe essentially without silicone oil or with a low amount of silicone oil.

In preferred embodiments, the syringe plunger stopper comprises an elastomer, preferably a thermoplastic elastomer, silicone polymer, or rubber.

A suitable syringe plunger stopper may additionally comprise a coating that reduces the gliding force needed for an injection using the syringe, wherein the additional coating is preferably not a silicone oil. A preferred coating in that context may comprise fluoropolymer coating, expanded fluoropolymer coating, or a silicone polymer coating.

In preferred embodiments, the syringe is configured for intramuscular injection, intradermal injection, intratumoral injection, intravenous injection, or intraocular injection (e.g. intravitreal injection). In some preferred embodiments, the syringe is configured for intramuscular injection.

In preferred embodiments, the syringe has a volume of about 0.01 ml to about 25 ml, preferably, 0.1 ml to about 10 ml, even more preferably about 0.1 ml to 2 ml, still more preferably 0.1 ml to about 1.0 ml.

Particular examples of syringes for injection are provided in the example section.

Particularly preferred syringes may be selected from the following materials or syringe systems, or combinations thereof:
Silicone-oil free syringe S1 (SOF-S1): Terumo Plajex 1 mL Long Luer Lock SOF Ref: PJ-B1LL2FTF1; PJ-R1 LNBM1 Plajex 1 mL Long Plunger Lot: 190517R1; Cyclo Olefin Polymer (COP) barrel. Supplier Teruma Europe.
Silicone-oil free syringe S2 (SOF-S2): SiO2 medical products; 1 mL Luer+OVS Quadlayer+Crosslinked; 1 mL Long Plunger NovaPure RU SP 4023/50G West Item 11402014; Cyclic olefin polymer (COP). Supplier SiO2 Materials Science.
Silicone-oil free syringe S3 (SOF-S3): BD PIR3-090 Hypak SCF1 mL PRT REF: 47406710; Hypak PR1 mL PSTYP Cristal lot: 974165 Ref: 47404008; Hypak TSCF1-3 mL 4023 Flur S Ref:47190510; glass barrel. Supplier BD Medical.

Further details regarding the syringe barrel and syringe plunger stopper of SOF-S1, SOF-S2 and SOF-S3 are provided in Tables A and B.

TABLE A
Preferred syringe barrels of SOF-S1, SOF-S2 and SOF-S3

| | Barrel | | |
|---|---|---|---|
| Syringe | Material | Coating | Lubrication |
| SOF-S1 | Cyclic olefin | None | Silicone oil- |
| | polymer | | free |
| SOF-S2 | Cyclic olefin | Cross-linked silicone coating | Silicone oil- |
| | polymer | with lubricant properties | free |
| SOF-S3 | Glass | None | Baked-on |
| | | | silicone |

TABLE B
Preferred syringe plunger stopper of SOF-S1, SOF-S2 and SOF-S3

| | Plunger stopper | | |
|---|---|---|---|
| Syringe | Material | Coating | Lubrication |
| SOF-S1 | Chlorobutyl | Silicone-based resin | Silicone oil-free |
| | | (i-coating ®) | |
| SOF-S2 | Bromobutyl | Fluorocarbon film | Silicone oil-free |
| | | (FluroTec ®) | |
| SOF-S3 | Bromobutyl | Fluorocarbon film | Silicone oil-free |

In preferred embodiments, upon storage in the syringe, less than 20%, 19%, 18%, 17%, 16%, 15%, 14%, 13%, 12%, 11%, 10%, 9%, 8%, 7%, 6%, 5%, 4%, 3% or 2% of the RNA of the pharmaceutical composition of the invention (e.g. component B) is agglomerated. In preferred embodiments, storage is for about 30 minutes to about 6 months, at a temperature in a range from about 5° C. to about 25° C., preferably at least 6 hours at 5° C.

Accordingly, it is preferred in the context of the invention that an exposure of the pharmaceutical composition (e.g. component B) with the syringe does not generate RNA agglomeration in the pharmaceutical composition.

In preferred embodiments, the syringe produces less than about 20%, 19%, 18%, 17%, 16%, 15%, 14%, 13%, 12%, 11%, 10%, 9%, 8%, 7%, 6%, 5%, 4%, 3% or 2% RNA agglomeration when incubated with an aqueous test formulation comprising RNA encapsulated in LNPs for 6 hours at 20° C. or when incubated with the pharmaceutical composition of the invention (e.g. component B) for 6 hours at 20° C.

As used herein, the term “aqueous test formulation” refers to a pharmaceutical composition comprising RNA encapsulated in LNPs. The aqueous test formulation comprises LNPs which have a molar ratio of approximately 47.4:10:40.9:1.7 proportion (mol %) of cationic lipid III-3, DSPC, cholesterol and PEG-lipid of formula (IVa) (with n=49 or with n=45). The LNPs encapsulate an RNA, wherein the RNA is the RNA according to SEQ ID NO: 5. The RNA comprises a 5′ Cap1 structure and does not comprise chemically modified nucleotides. The RNA integrity is at least about 80% (as determined using RP(HPLC)) and the encapsulation efficiency is at least 80% (as determined by a Ribogreen assay) and the Z-average particle size is in a range of about 60 nm to about 115 nm (as determined by DLS). N/P ratio of the LNPs to the RNA in the aqueous test formulation is about 6, and the wt/wt ratio of lipid to the RNA in the aqueous test formulation is about 25:1. Typically, the aqueous test formulation comprising LNPs encapsulating an RNA is obtainable by Tee-piece based formulation, preferably according to Example 1.4.

Preferably, the volume of the aqueous test formulation is adjusted to the typically used syringe volume. Preferably, the respective volume of aqueous test formulation is drawn into the syringe through the needle and subsequently incubated for 6 hours at 20° C. Preferably, 0.5 mL of aqueous test formulation is drawn into the syringe through the needle and subsequently incubated in the needle barrel for 6 hours at 20° C.

The term “RNA agglomeration” as used herein generally describes RNA molecules that are accumulated or clumped to form aggregates. RNA agglomerates can comprise more than one RNA molecule and/or a different molecule compound. Without wishing to be bound to theory, RNA agglomeration in the context of the invention can be caused by commonly used lubricant oils of syringes e.g. silicone oils. Accordingly, RNA agglomerates may additionally comprise lubricant oils, e.g. silicone oils.

“RNA agglomeration” in percent (%) as used herein has to be understood as the ratio of RNA agglomerates in an RNA sample, preferably detectable in an analytical HPLC. Atypical HPLC chromatogram of an RNA comprises a lead/fronting fraction (corresponding to short RNA fragments), a main fraction (corresponding to the expected full length RNA, e.g. the RNA with about 100% integrity), and a tail fraction (the RNA with a longer elution time that the RNA with the correct RNA length, e.g. comprising the RNA fraction potentially comprising RNA agglomeration). In case HPLC is used (e.g. RP-HPLC), the analysis of the RNA agglomeration may be based on determining the relative peak area of the tail fractions in a corresponding chromatogram. The relative peak area of the tail fractions may be determined by any suitable software which evaluates the signals of the detector system. The process of determining the relative peak area of the tail fractions is also referred to as integration. The relative peak area of the tail representing the fraction of RNA agglomeration is typically set in relation to the peak area of the total RNA in a respective sample. To arrive at a respective RNA agglomeration value, the peak areas of the tail in % of a corresponding control sample (the same composition not exposed to a syringe) has to be subtracted. The RNA agglomeration may therefore be expressed as % RNA agglomeration.

Accordingly, in preferred embodiments, the RNA agglomeration is measured using analytical (RP)HPLC (UV 260 nm) of RNA isolated from the components of the aqueous test formulation or the pharmaceutical composition of the invention (e.g. component B).

In preferred embodiments, the RNA agglomeration in % is determined based on the proportion of the relative peak area of the tail in the obtained chromatogram, e.g. the obtained HPLC chromatogram. The relative peak area of the tail represents the fraction of RNA agglomeration that is set in relation to the peak area of the total RNA in a respective sample.

In the context of the invention, RNA agglomeration is determined using analytical (RP)HPLC. Typically, a test sample of the pharmaceutical composition or the aqueous test formulation may be treated with a detergent (e.g. about 2% Triton X100) to dissociate the lipid based carrier (if used as a formulation) and to release the encapsulated RNA. In embodiments where the RNA is formulated in cationic or polycationic peptides of proteins, the RNA may be treated with e.g. heparin. The released RNA (also referred to as “RNA isolated from the components of the aqueous test formulation or the pharmaceutical composition of the invention”) may be captured using suitable binding compounds, e.g. Agencourt AMPure XP beads (Beckman Coulter, Brea, CA, USA) essentially according to the manufacturer's instructions. Following preparation of the RNA sample, analytical (RP)HPLC may be performed to determine the amount of RNA agglomeration.

Typically, for determining RNA agglomeration, the RNA samples may be diluted to e.g. an RNA concentration of 0.05 g/I using e.g. water for injection (WFI). A certain volume (e.g. 10 μl) of the diluted RNA sample may be injected into an HPLC column (e.g. a monolithic poly(styrene-divinylbenzene), e.g. 4.6 mm×50 mm). Analytical (RP)HPLC may be performed using the following conditions: Gradient 1: Buffer A (0.1 M TEAA (pH 7.0); Buffer B (0.1 M TEAA (pH 7.0) containing 25% acetonitrile. Starting at 35% buffer B followed by an extension to 55% buffer B over 20 minutes at a flow rate of 1 ml/min (70° C. column temperature). HPLC chromatograms are typically recorded at a wavelength of 260 nm. A typical HPLC chromatogram of an RNA comprises a lead/fronting fraction, a main fraction (corresponding to the expected full length RNA), and a tail fraction (representing the RNA fraction with RNA agglomeration). The obtained chromatograms may be evaluated using a software and the relative peak area of the tail fractions corresponding to the agglomerated RNA may be determined in percent (%) as described herein. The relative area of the tail fractions in the chromatogram indicates the amount of RNA agglomeration, e.g. the amount of RNA that has a longer elution time than the RNA with the expected size. Since the amount of the RNA injected into the HPLC is typically known, the analysis of the relative peak area of the tail fractions provides information on the amount of RNA agglomeration. That value may be used to calculate an agglomerated value (RNA agglomeration [%]). To arrive at a respective RNA agglomeration value, the peak areas of the tail in % of a corresponding control sample (the same composition not exposed to a syringe) has to be subtracted. For example, if the relative peak area of the tail in the control sample is 10%, and the relative peak area of the tail in the sample subjected to a syringe is 30%, the calculated RNA agglomeration value is 20%. That value is indicated as “RNA agglomeration in %”.

In preferred embodiments, analytical (RP)HPLC is performed on an analytical monolithic poly(styrene-divinylbenzene column. In preferred embodiments, RNA agglomeration in % is determined as described in Example 2.

In preferred embodiments, the syringe produces less than 10 mAU*min, 9 mAU*min, 9 mAU*min, 7 mAU*min, 6 mAU*min, 5 mAU*min of detectable compounds in 20 μl of a 2-Propanol extract as determined by analytical RP-HPLC (UV 260 nm).

Typically, for determining compounds in 2-Propanol extracts, 20 μl of a 2-Propanol extract is injected into an HPLC column (e.g. an analytic C18 column). Analytical (RP)HPLC may be performed using the following conditions:
Gradient 1: Buffer A: 0.1 M TEAA (pH 7.0); Buffer B: acetonitrile/methanol (50%/50%), 0.1% NH4OH.

Starting at 10% buffer B, holding 10% B for 1 min, following to 100% buffer B in 15 min. Additional holding for 6 min at 100% buffer B, followed by decreasing to 10% buffer B. Flow rate of 0.5 ml/min (50° C. column temperature).

HPLC chromatograms are typically recorded at a wavelength of 260 nm. The obtained chromatograms may be evaluated using a software and the total area of detectable compounds may be determined (mAU*min). The total area of detectable compounds in the chromatogram expressed as “mAU*min” indicates the amount of detectable compounds that have been extracted from the syringe (compounds that can potentially cause RNA agglomeration). Such detectable compounds comprise extractable compounds that are soluble in organic solvents, e.g. lubricant oils comprising silicone oil. For determining the identity of extracted compounds, methods such as mass spectrometry may be used.

In preferred embodiments, the analytical RP-HPLC (UV 260 nm) for analyzing the 2-Propanol extract is performed on a C18 modified analytical HPLC column, preferably a BEH C18 column. In preferred embodiments, the 2-Propanol extract is determined as described in Example 3.

In preferred embodiments, the 2-Propanol extract is obtained by repeated draw/eject cycles using 1 mL 2-Propanol at room temperature. Preferably, the draw/eject procedure is repeated three times.

Accordingly, to generate a 2-Propanol extract for determining compounds that may cause RNA aggregation, 1 mL 2-Propanol (isopropanol) is drawn into a syringe (through the needle), followed by ejection of the full volume of 2-Propanol into a vial. The ejected 1 mL 2-Propanol is again drawn from the vial into the syringe (through the needle), followed by another ejection into a vial. The ejected 2-Propanol is again drawn from the vial into the syringe ((through the needle) and ejected into a vial. The obtained 2-Propanol extract (3 times extracted) is then analyzed using RP-HPLC as explained above. The extraction procedure as described herein ensures that all parts of the syringe that are in contact with the pharmaceutical composition, e.g. needle, syringe barrel, plunger stopper, are extracted with the organic solvent.

Accordingly, in preferred embodiments, upon storage in the syringe of the invention (e.g. component A), the pharmaceutical composition of the invention (e.g. component B) or the aqueous test formulation comprising RNA encapsulated in LNPs is essentially free of silicone oils, lubricants oils or extractable lubricant oils that are soluble in an organic solvent. In preferred embodiments, storage is for about 30 minutes to about 6 months, at a temperature in a range from about 5° C. to about 25° C., more preferably at least 6 hours at 5° C.

As specified herein, the pharmaceutical composition of the invention, e.g. the pharmaceutical composition of the kit or kit of parts of the first aspect (herein also referred to as component B), or the pre-filled syringe of the second aspect comprises RNA.

In various embodiments, the pharmaceutical composition of the invention (e.g. component B) comprises a certain concentration of RNA.

The concentration of the RNA in the pharmaceutical composition of the invention (e.g. component B) may be in a range of about 0.1 μg/ml to about 10 mg/ml, 0.1 μg/ml to about 5 mg/ml, 0.1 μg/ml to about 2 mg/ml, 0.1 μg/ml to about 1 mg/ml, or 0.1 μg/ml to about 500 μg/ml.

In preferred embodiments, the concentration of the RNA in the pharmaceutical composition of the invention (e.g. component B) is in a range of about 0.1 μg/ml to about 500 μg/ml, preferably in a range of about 0.1 μg/ml to about 100 μg/ml, more preferably in a range of about 1 μg/ml to about 100 μg/ml.

In embodiments, the concentration of the RNA in the pharmaceutical composition of the invention (e.g. component B) is for example about 1 μg/ml, about 2 μg/ml, about 3 μg/ml, about 4 μg/ml, about 5 μg/ml, about 10 μg/ml, about 20 μg/ml, about 30 μg/ml, about 40 μg/ml, about 50 μg/ml.

Without whishing to be bound to theory, the observed effect of RNA agglomeration may depend on the concentration of RNA in the composition, wherein the problem of RNA agglomeration increases with decreasing RNA concentrations.

Accordingly, the invention as provided herein may particularly suitable or advantageous for pharmaceutical compositions comprising low-concentrations or RNA.

Accordingly, in preferred embodiments, the concentration of the RNA in the pharmaceutical composition of the invention (e.g. component B) is lower than 200 μg/ml, preferably lower than at least 100 μg/ml, more preferably lower than 50 μg/ml.

In various embodiments, the RNA of the pharmaceutical composition of the invention (e.g. component B) has a certain RNA integrity.

The term “RNA integrity” generally describes whether the complete RNA sequence with the correct RNA length is present in the pharmaceutical composition. Low RNA integrity could be due to, amongst others, RNA agglomeration, RNA degradation, RNA cleavage, incorrect or incomplete chemical synthesis of the RNA, incorrect base pairing, integration of modified nucleotides or the modification of already integrated nucleotides, lack of capping or incomplete capping, lack of polyadenylation or incomplete polyadenylation, or incomplete RNA in vitro transcription. RNA is a fragile molecule that can easily degrade, which may be caused e.g. by temperature, ribonucleases, pH or other factors (e.g. nucleophilic attacks, hydrolysis etc.), which may reduce the RNA integrity and, consequently, the functionality of the RNA.

The skilled person can choose from a variety of different chromatographic or electrophoretic methods for determining an RNA integrity. Chromatographic and electrophoretic methods are well-known in the art. In case chromatography is used (e.g. RP-HPLC), the analysis of the integrity of the RNA may be based on determining the peak area (or “area under the peak”) of the expected full length RNA (the RNA with the correct RNA length) in a corresponding chromatogram. A typical HPLC chromatogram of an RNA comprises a lead/fronting fraction (corresponding to short RNA fragments), a main fraction (corresponding to the expected full length RNA, e.g. the RNA with about 100% integrity), and a tail fraction (the RNA with a longer elution time that the RNA with the correct RNA length, e.g. representing the RNA fraction with RNA agglomeration). The main peak area may be determined by any suitable software which evaluates the signals of the detector system. The process of determining the main peak area is also referred to as integration. The main peak area representing the full length RNA (the RNA with the correct RNA length) is typically set in relation to the peak area of the total RNA in a respective sample. For example, RNA species that are shorter than the expected size (lead/fronting fraction, e.g. abortive RNA sequences) and RNA sequences that are longer than the expected size (tail fraction, e.g. RNA agglomerates) can reduce the RNA integrity value. The RNA integrity may be expressed in % RNA integrity.

In the context of the invention, RNA integrity may be determined using analytical (RP)HPLC. Typically, a test sample of the pharmaceutical composition comprising RNA, e.g. lipid based carrier encapsulating RNA, may be treated with a detergent (e.g. about 2% Triton X100) to dissociate the lipid based carrier and to release the encapsulated RNA. In embodiments where the RNA is formulated in cationic or polycationic peptides of proteins, the RNA may be treated with e.g. heparin. The released RNA may be captured using suitable binding compounds, e.g. Agencourt AMPure XP beads (Beckman Coulter, Brea, CA, USA) essentially according to the manufacturer's instructions. Following preparation of the RNA sample, analytical (RP)HPLC may be performed to determine the integrity of RNA. Typically, for determining RNA integrity, the RNA samples may be diluted to an RNA concentration of 0.05 g/I using e.g. water for injection (WFI). About 10 μl of the diluted RNA sample may be injected into an HPLC column (e.g. a monolithic poly(styrene-divinylbenzene), e.g. 4.6 mm×50 mm). Analytical (RP)HPLC may be performed using the following conditions: Gradient 1: Buffer A (0.1 M TEAA (pH 7.0)); Buffer B (0.1 M TEAA (pH 7.0) containing 25% acetonitrile). Starting at 35% buffer B followed by an extension to 55% buffer B over 20 minutes at a flow rate of 1 ml/min (70° C. column temperature). HPLC chromatograms are typically recorded at a wavelength of 260 nm. A typical HPLC chromatogram of an RNA comprises a lead/fronting fraction, a main fraction (corresponding to the expected full length RNA), and a tail fraction (corresponding to the RNA fraction with RNA agglomeration). The obtained chromatograms may be evaluated using a software and the relative peak area of the main fraction corresponding to the expected RNA may be determined in percent (%) as commonly known in the art. The relative peak area of the main fraction indicates the amount of RNA that has 100% RNA integrity, e.g. the amount of RNA that has the expected size. Since the amount of the RNA injected into the HPLC is typically known, the analysis of the relative peak area of the main fraction provides information on the integrity of the RNA. Thus, if e.g. 100 ng RNA have been injected in total, and 100 ng are determined as the relative peak area of the main fraction, the RNA integrity would be 100%. If, for example, the relative peak area of the main fraction would correspond to 80 ng, and 20 ng would correspond to later eluting RNA agglomerates (e.g. the tail fraction), the RNA integrity would be 80%. Accordingly, RNA integrity in the context of the invention is determined using analytical HPLC, preferably analytical RP-HPLC.

In embodiments, the RNA of the pharmaceutical composition of the invention (e.g. component B) has an RNA integrity ranging from about 40% to about 100%. In embodiments, the RNA has an RNA integrity ranging from about 50% to about 100%. In embodiments, the RNA has an RNA integrity ranging from about 60% to about 100%. In embodiments, the RNA has an RNA integrity ranging from about 70% to about 100%. In embodiments, the RNA integrity is for example about 50%, about 60%, about 70%, about 80%, or about 90%. RNA is suitably determined using analytical HPLC, preferably analytical RP-HPLC.

In preferred embodiments, the RNA of the pharmaceutical composition of the invention (e.g. component B) has an RNA integrity of at least about 50%, preferably of at least about 60%, more preferably of at least about 70%, most preferably of at least about 80%. RNA is suitably determined using analytical HPLC, preferably analytical RP-HPLC.

In preferred embodiments, the RNA of the pharmaceutical composition of the invention (e.g. component B) has an RNA integrity of at least about 50%, preferably of at least about 60%, more preferably of at least about 70%, most preferably of at least about 80% upon storage or exposure to the syringe (e.g. component A). RNA is suitably determined using analytical HPLC, preferably analytical RP-HPLC as described herein. In preferred embodiments, storage is for about 30 minutes to about 6 months, at a temperature in a range from about 5° C. to about 25° C., more preferably at least 6 hours at 5° C.

For calculating the effect of the syringe on RNA integrity, RNA integrity values obtained after syringe incubation may be subtracted with the RNA integrity values of a control (not incubated in a syringe). That value is indicated as “Delta RNA integrity in %”. For example, the value “12” means for the respective syringe, that the RNA integrity is 12% lower than the RNA integrity of the control.

In preferred embodiments, the syringe of component A produces less than about 20%, 19%, 18%, 17%, 16%, 15%, 14%, 13%, 12%, 11%, 10%, 9%, 8%, 7%, 6%, 5%, 4%, 3% or 2% reduction in RNA integrity incubated with an aqueous test formulation comprising RNA encapsulated in LNPs, preferably for 6 hours at 20° C. (also referred to as Delta RNA integrity in %). In preferred embodiments, the syringe of component A produces less than about 20%, 19%, 18%, 17%, 16%, 15%, 14%, 13%, 12%, 11%, 10%, 9%, 8%, 7%, 6%, 5%, 4%, 3% or 2% reduction in RNA integrity when incubated with the pharmaceutical composition of component B, preferably for 6 hours at 20° C. (also referred to as Delta RNA integrity in %).

In various embodiments, RNA of the pharmaceutical composition of the invention (e.g. component B) does not exceed a certain proportion of free RNA.

The term “free RNA” or “non-complexed RNA” or “non-encapsulated RNA” comprise the RNA molecules that are not formulated, e.g. encapsulated in a lipid-based carrier (as specified below). During formulation of the pharmaceutical composition, free RNA may represent a contamination or an impurity. A large proportion of non-encapsulated or free RNA may also be an indicator for destabilization of the formulation, e.g. destabilization of lipid-based carriers. Free RNA may be prone to RNA agglomeration produced by the syringe.

The skilled person can choose from a variety of different methods for determining the amount and/or the proportion of free RNA in the pharmaceutical composition. Free RNA in the pharmaceutical composition may be determined by chromatographic methods (e.g. AEX, SEC) or by using probes (e.g. dyes) that bind to free RNA in the composition. In the context of the invention, the amount of free RNA or non-encapsulated RNA may be determined using a dye based assay. Suitable dyes that may be used to determine the amount and/or the proportion of free RNA comprise RiboGreen®, PicoGreen® dye, OliGreen® dye, QuantiFluor® RNA dye, Qubit® RNA dye, Quant-iT™ RNA dye, TOTO®-1 dye, YOYO®-1 dye. Such dyes are suitable to discriminate between free RNA and encapsulated RNA. Reference standards consisting of defined amounts of free RNA or encapsulated RNA may be used and mixed with the respective reagent (e.g. RiboGreen® reagent (Excitation 500 nm/Emission 525 nm)) as recommended by the supplier's instructions. Typically, the free RNA of the pharmaceutical composition is quantitated using the Quant-iT RiboGreen RNA Reagent according to the manufacturer's instructions. The proportion of free RNA in the context of the invention is typically determined using a RiboGreen assay.

In embodiments, the pharmaceutical composition of the invention (e.g. component B) comprises less than about 20% free RNA, preferably less than about 15% free RNA, more preferably less than about 10% free RNA, most preferably less than about 5% free RNA.

In preferred embodiments, the pharmaceutical composition of the invention (e.g. component B) comprises RNA formulated in lipid-based carriers (as specified in detail below), wherein the lipid-based carriers encapsulate the RNA. Accordingly, in preferred embodiments, the pharmaceutical composition of the invention (e.g. component B) comprises lipid-based carriers that encapsulate the RNA.

The term “encapsulated RNA” comprise the RNA molecules that are encapsulated in the lipid-based carriers as defined herein. The proportion of encapsulated RNA in the context of the invention is typically determined using a RiboGreen assay.

Accordingly, in various embodiments, about 70% to about 100% of the RNA in the pharmaceutical composition of the invention (e.g. component B) is encapsulated in the lipid-based carriers. In embodiments, the pharmaceutical composition comprises about 80% encapsulated RNA (and about 20% free RNA), about 85% encapsulated RNA (and about 15% free RNA), about 90% encapsulated RNA (and about 10% free RNA), or about 95% encapsulated RNA (and 5% about free RNA).

In various embodiments, the pharmaceutical composition of the invention (e.g. component B) comprises purified RNA. It may be suitable to apply certain purification steps during RNA production to achieve certain RNA purity levels in regards of various impurities. Accordingly, the RNA used for formulation has been purified (before formulation/encapsulation) to remove various RNA impurities. The various RNA purification steps (e.g. RP-HPLC, tangential flow filtration) may be employed to remove various contaminations including divalent metal ions. Suitably, the RNA used for formulation/encapsulation in the lipid-based carriers has been purified to remove divalent metal ions.

In embodiments, the RNA of the pharmaceutical composition is a purified RNA.

The term “purified RNA” or “purified mRNA” as used herein has to be understood as RNA which has a higher purity after certain purification steps (e.g. HPLC, TFF, Oligo d(T) purification, precipitation, filtration, AEX, cellulose purification) than the starting material (e.g. crude in vitro transcribed RNA). Typical impurities that are essentially not present in purified RNA comprise peptides or proteins (e.g. enzymes derived from RNA in vitro transcription, e.g. RNA polymerases, RNases, pyrophosphatase, restriction endonuclease, DNase), spermidine, BSA, short abortive RNA sequences, RNA fragments (short double stranded RNA fragments, short single stranded RNA fragments, abortive RNA sequences etc.), free nucleotides (modified nucleotides, conventional NTPs, cap analogue), template DNA fragments, buffer components (HEPES, TRIS, MgCl2, CaCl2) etc. Other potential impurities may be derived from e.g. fermentation procedures and comprise bacterial impurities (bioburden, bacterial DNA, bacterial RNA) or impurities derived from purification procedures (organic solvents etc.). Accordingly, it is desirable in this regard for the “degree of RNA purity” to be as close as possible to 100%.

Accordingly, “purified RNA” as used herein has a degree of purity of more than 75%, 80%, 85%, very particularly 90%, 91%, 92%, 93%, 94%, 95%, 96%, 97%, 98% and most favourably 99% or more. The degree of purity may for example be determined by an analytical HPLC, wherein the percentages provided above correspond to the ratio between the area of the peak for the target RNA and the total area of all peaks representing all the by-products. Alternatively, the degree of purity may for example be determined by an analytical agarose gel electrophoresis or capillary gel electrophoresis.

In embodiments, the RNA of the pharmaceutical composition is an RP-HPLC purified RNA and/or a tangential flow filtration (TFF) purified RNA.

In preferred embodiments, the RNA of pharmaceutical composition is an RP-HPLC purified RNA, wherein the RNA has been purified using a method as described in published patent application WO2008/077592, the specific disclosure relating to the published PCT claims 1 to 28 herewith incorporated by reference.

In embodiments, the RNA of the pharmaceutical composition has been purified by at least one step of TFF against a salt buffer, preferably against an NaCl buffer. In preferred embodiments, a tangential flow filtration method as described in published patent application WO2016/193206 may be used, the specific disclosure relating to the published PCT claims 1 to 48 herewith incorporated by reference.

In preferred embodiments, the RNA of the pharmaceutical composition is an artificial RNA.

The term “artificial RNA” as used herein is intended to refer to an RNA that does not occur naturally. In other words, an artificial RNA may be understood as a non-natural RNA molecule. Such RNA molecules may be non-natural due to its individual sequence (e.g. G/C content modified coding sequence, heterologous UTRs) and/or due to other modifications, e.g. structural modifications of nucleotides. Typically, artificial RNA may be designed and/or generated by genetic engineering to correspond to a desired artificial sequence of nucleotides. In this context, an artificial RNA is a sequence that may not occur naturally, i.e. a sequence that differs from the wild type sequence/the naturally occurring sequence by at least one nucleotide (via e.g. codon modification as further specified below). The term “artificial RNA” is not restricted to mean “one single molecule” but is understood to comprise an ensemble of essentially identical RNA molecules. Accordingly, the term may relate to a plurality of essentially identical RNA molecules.

In various embodiments, the RNA of the pharmaceutical composition does not comprise (chemically) modified nucleotides.

In the context of the invention, the terms “modified nucleotides” or “chemically modified nucleotides” do not encompass 5′ cap structures (e.g. cap0, cap1 as defined herein). Additionally, the term “modified nucleotides” does not relate to modifications of the codon usage of e.g. a respective coding sequence. The terms “modified nucleotides” or “chemically modified nucleotides” do encompass all potential natural and non-natural chemical modifications of the building blocks of an RNA, namely the ribonucleotides A, G, C, U, with the exception of the natural cap0, cap1 or cap2 structure.

According to preferred embodiments, the RNA of the pharmaceutical composition of the invention (e.g. component B) consists of non-modified A, U, G, and C ribonucleotides, optionally comprising a 5′cap structure (e.g. a cap1 structure).

Accordingly, the RNA of the pharmaceutical composition is not a (chemically)modified RNA, wherein the modification may refer to chemical modifications comprising backbone modifications as well as sugar modifications or base modifications.

A chemically modified RNA may comprise nucleotide analogues/modifications, e.g. backbone modifications, sugar modifications or base modifications. A backbone modification is a chemical modification in which phosphates of the backbone of the nucleotides of the RNA are modified. A sugar modification is a chemical modification of the sugar of the nucleotides of the RNA. Furthermore, a base modification is a chemical modification of the base moiety of the nucleotides of the RNA.

In preferred embodiments, the RNA of the pharmaceutical composition does not comprise chemically modified nucleotides selected from pseudouridine (ψ), N1-methylpseudouridine (m1ψ), N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine.

In alternative and equally preferred embodiments, the RNA of the pharmaceutical composition comprises chemically modified nucleotides preferably selected from pseudouridine (ψ), N1-methylpseudouridine (m1ψ), N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine.

In such embodiments, essentially all, e.g. essentially 100% of the uracil in the coding sequence of the RNA have a chemical modification, preferably a chemical modification that is in the 5-position of the uracil. Incorporating modified nucleotides such as e.g. pseudouridine (ψ), N1-methylpseudouridine (m1ψ), 5-methylcytosine, and/or 5-methoxyuridine into the coding sequence may be advantageous as unwanted innate immune responses (upon administration of the composition) may be adjusted or reduced (if required). Accordingly, in embodiments, the RNA of the pharmaceutical composition comprises modified nucleotides selected from pseudouridine (ψ), N1-methylpseudouridine (m1ψ), 5-methylcytosine, and/or 5-methoxyuridine.

Particularly preferred chemically modified nucleotides in the context of the invention are pseudouridine (ψ) or N1-methylpseudouridine (m1ψ).

In preferred embodiments, the RNA of the pharmaceutical composition is an in vitro transcribed RNA, preferably wherein RNA in vitro transcription has been performed in the presence of a sequence optimized mixture of, preferably, (chemically) non-modified nucleotides (that are A, U, G, and C ribonucleotides) and, optionally, in the presence of a cap analogue (e.g. a cap1 antilog).

In embodiments, the RNA of the pharmaceutical composition of the invention (e.g. component B) has a length ranging from about 100 nucleotides to about 10000 nucleotides, preferably ranging from about 500 nucleotides to about 10000 nucleotides, more preferably ranging from about 1000 nucleotides to about 10000 nucleotides.

In embodiments, the RNA of the pharmaceutical composition of the invention (e.g. component B) has a length ranging from about 100 nucleotides to about 5000 nucleotides, preferably ranging from about 500 nucleotides to about 5000 nucleotides, more preferably ranging from about 1000 nucleotides to about 5000 nucleotides.

Without whishing to be bound to theory, the observed effect of RNA agglomeration may depend on the length of the RNA of the composition, wherein the problem of RNA agglomeration increases with increasing RNA length.

Accordingly, the invention as provided herein may particularly suitable or advantageous for pharmaceutical compositions comprising long chain RNA (e.g. RNA that is at least about 100 nucleotides in length).

Accordingly, in preferred embodiments, the RNA of the pharmaceutical composition of the invention (e.g. long chain RNA of component B) is at least about 100 nucleotides in length, 500 nucleotides in length, preferably the RNA is at least 1000 nucleotides in length, more preferably the RNA is at least 1500 nucleotides in length.

In various embodiments, the RNA of the pharmaceutical composition is a therapeutic RNA.

The term “therapeutic RNA” relates to an RNA providing a therapeutic modality. The term “therapeutic” in that context has to be understood as “providing a therapeutic function” or as “being suitable for therapy or administration”. However, “therapeutic” in that context should not at all to be understood as being limited to a certain therapeutic modality. Examples for therapeutic modalities may be the provision of a coding sequence (via said therapeutic RNA) that encodes for a peptide or protein (wherein said peptide or protein has a certain therapeutic function, e.g. an antigen for a vaccine, or an enzyme for protein replacement therapies). A further therapeutic modality may be genetic engineering, wherein the RNA provides or orchestrates factors to e.g. manipulate DNA and/or RNA in a cell or a subject. Typically, the term “therapeutic RNA” does not include natural RNA extracts or RNA preparations (e.g. obtained from bacteria, or obtained from plants) that are not suitable for administration to a subject (e.g. animal, human). For being suitable for a therapeutic purpose, the RNA of the invention may be an artificial RNA.

In embodiments, the RNA of the pharmaceutical composition, preferably the therapeutic RNA, is selected from viral RNA, retroviral RNA, replicon RNA, small interfering RNA (siRNA), antisense RNA, saRNA (small activating RNA), CRISPR RNA (small guide RNA, sgRNA), ribozymes, aptamers, riboswitches, immunostimulating RNA, transfer RNA (tRNA), ribosomal RNA (rRNA), small nuclear RNA (snRNA), small nucleolar RNA (snoRNA), microRNA (miRNA), Piwi-interacting RNA (piRNA), self-replicating RNA, circular RNA, or mRNA.

In embodiments, the RNA of the pharmaceutical composition, preferably the therapeutic RNA, is a non-coding RNA, preferably a CRISPR/Cas9 guide RNA or a small interfering RNA (siRNA).

As used herein, the term “guide RNA” (gRNA) relates to an RNA molecule capable of targeting a CRISPR-associated protein or a CRISPR-associated endonuclease to a target DNA sequence of interest. The term guide RNA has to be understood in its broadest sense, and may comprise two-molecule gRNAs (“tracrRNA/crRNA”) comprising crRNA (“CRISPR RNA” or “targeter-RNA” or “crRNA” or “crRNA repeat”) and a corresponding tracrRNA (“trans-acting CRISPR RNA” or “activator-RNA” or “tracrRNA”) molecule, or single-molecule gRNAs. A “sgRNA” typically comprises a crRNA connected at its 3′ end to the 5′ end of a tracrRNA through a “loop” sequence.

In preferred embodiments, the RNA of the pharmaceutical composition is not an RNA for RNA interference (RNAi). In particular, the RNA of the pharmaceutical composition is not an siRNA.

In preferred embodiments, the RNA of the pharmaceutical composition is a single stranded RNA.

In preferred embodiments, the RNA of the pharmaceutical composition is a coding RNA.

A coding RNA can be any type of RNA construct (for example a double stranded RNA, a single stranded RNA, a circular double stranded RNA, or a circular single stranded RNA) characterized in that said coding RNA comprises at least one coding sequence (cds) that is translated into at least one amino-acid sequence (upon administration to e.g. a cell).

Most preferably, said coding RNA may be selected from an mRNA, a (coding) self-replicating RNA, a (coding) circular RNA, a (coding) viral RNA, or a (coding) replicon RNA.

In embodiments, the RNA of the pharmaceutical composition is a circular RNA. As used herein, the terms “circular RNA” or “circRNAs” have to be understood as a circular polynucleotide constructs that may encode at least one peptide or protein.

Preferably, such a circRNA is a single stranded RNA molecule. In preferred embodiments, said circRNA comprises at least one coding sequence encoding at least one peptide or protein as defined herein, or a fragment or variant thereof.

In embodiments, the RNA of the pharmaceutical composition is a replicon RNA. The term “replicon RNA” is e.g. intended to be an optimized self-replicating RNA. Such constructs may include replicase elements derived from e.g. alphaviruses (e.g. SFV, SIN, VEE, or RRV) and the substitution of the structural virus proteins with the nucleic acid of interest (that is, the coding sequence encoding an antigenic peptide or protein as defined herein). Alternatively, the replicase may be provided on an independent coding RNA construct or a coding DNA construct. Downstream of the replicase may be a sub-genomic promoter that controls replication of the replicon RNA.

In particularly preferred embodiments, the RNA of the pharmaceutical composition of the invention (e.g. component B) is an mRNA. Preferably, the mRNA of the invention is not a replicon RNA and not a self-replicating RNA.

The terms “RNA” and “mRNA” are e.g. intended to be a ribonucleic acid molecule, i.e. a polymer consisting of nucleotides.

These nucleotides are usually adenosine-monophosphate, uridine-monophosphate, guanosine-monophosphate and cytidine-monophosphate monomers which are connected to each other along a so-called backbone. The backbone is formed by phosphodiester bonds between the sugar, i.e. ribose, of a first and a phosphate moiety of a second, adjacent monomer. The specific succession of the monomers is called the RNA-sequence. The mRNA (messenger RNA) provides the nucleotide coding sequence that may be translated into an amino-acid sequence of a particular peptide or protein.

Accordingly, in preferred embodiments, the RNA of the pharmaceutical composition comprises at least one coding sequence.

In embodiments, the length the coding sequence (of the RNA) may be at least or greater than about 200, 250, 300, 350, 400, 450, 500, 600, 700, 800, 900, 1000, 1200, 1400, 1600, 1800, 2000, 2500, 3000, 3500, 4000, 5000, or 6000 nucleotides. In embodiments, the length of the coding sequence may be in a range of from about 500 to about 2000 nucleotides.

In preferred embodiments, the RNA of the pharmaceutical composition comprises at least one codon modified coding sequence.

In preferred embodiments, the at least one coding sequence of the RNA is a codon modified coding sequence. Suitably, the amino acid sequence encoded by the at least one codon modified coding sequence is not being modified compared to the amino acid sequence encoded by the corresponding wild type or reference coding sequence.

The term “codon modified coding sequence” relates to coding sequences that differ in at least one codon (triplets of nucleotides coding for one amino acid) compared to the corresponding wild type or reference coding sequence. Suitably, a codon modified coding sequence in the context of the invention may show improved resistance to in vivo degradation and/or improved stability in vivo, and/or improved translatability in vivo and/or improved temperature stability upon storage. Codon modifications in the broadest sense make use of the degeneracy of the genetic code wherein multiple codons may encode the same amino acid and may be used interchangeably to optimize/modify the coding sequence for in vivo applications as outlined above.

In preferred embodiments, the at least one coding sequence of the RNA is a codon modified coding sequence, wherein the codon modified coding sequence is selected from C maximized coding sequence (according to WO2015/062738), codon adaptation index (CAI) maximized coding sequence, human codon usage adapted coding sequence, G/C content modified coding sequence, and G/C optimized coding sequence, or any combination thereof.

In preferred embodiments, the RNA of the pharmaceutical composition comprises a codon modified coding sequence, wherein the G/C content of the coding sequence is optimized compared to the G/C content of the corresponding wild type or reference coding sequence (herein referred to as “G/C content optimized coding sequence”). “Optimized” in that context refers to a coding sequence wherein the G/C content is preferably increased to the essentially highest possible G/C content. The amino acid sequence encoded by the G/C content optimized coding sequence of the RNA is preferably not modified as compared to the amino acid sequence encoded by the respective wild type or reference coding sequence. The generation of a G/C content optimized RNA sequences may be carried out using a method according to WO2002/098443. In this context, the disclosure of WO2002/098443 is included in its full scope in the present invention. Advantageously, RNA sequences having an increased G/C content may be more stable or may show a better expression than sequences having an increased A/U.

Suitably, the G/C content of the coding sequence of the RNA of the pharmaceutical composition is increased by at least 10%, 20%, 30%, preferably by at least 40% compared to the G/C content of the corresponding wild type or reference coding sequence.

In various embodiments, the RNA of the pharmaceutical composition of the invention (e.g. component B) has a GC content of about 50% to about 80%. In preferred embodiments, the RNA of the pharmaceutical composition has a GC content of at least about 50%, preferably at least about 55%, more preferably of at least about 60%. In specific embodiments, the RNA of the pharmaceutical composition has a GC content of about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, or about 70%.

In various embodiments, the coding sequence of the RNA has a GC content of about 60% to about 90%. In preferred embodiments, the coding sequence of the RNA has a GC content of at least about 60%, preferably at least about 65%, more preferably of at least about 70%. In specific embodiments, the RNA of the composition has a GC content of about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, or about 80%.

In various embodiments, the RNA of the pharmaceutical composition comprises a 5′-cap structure, preferably a cap1 structure.

Accordingly, in preferred embodiments, the RNA of the pharmaceutical composition comprises a 5′-cap structure, preferably m7G, cap0, cap1, cap2, a modified cap0 or a modified cap1 structure.

The term “5′-cap structure” as used herein is intended to refer to the 5′ structure of the RNA, particularly a guanine nucleotide, positioned at the 5′-end of an RNA, e.g. an mRNA. Preferably, the 5′-cap structure is connected via a 5′-5′-triphosphate linkage to the RNA. Notably, a “5′-cap structure” or a “cap analogue” is not considered to be a “modified nucleotide” or “chemically modified nucleotides” in the context of the invention. 5′-cap structures which may be suitable in the context of the present invention are cap0 (methylation of the first nucleobase, e.g. m7GpppN), cap1 (additional methylation of the ribose of the adjacent nucleotide of m7GpppN), cap2 (additional methylation of the ribose of the 2nd nucleotide downstream of the m7GpppN), cap3 (additional methylation of the ribose of the 3rd nucleotide downstream of the m7GpppN), cap4 (additional methylation of the ribose of the 4th nucleotide downstream of the m7GpppN), ARCA (anti-reverse cap analogue), modARCA (e.g. phosphothioate modARCA), inosine, N1-methyl-guanosine, 2′-fluoro-guanosine, 7-deaza-guanosine, 8-oxo-guanosine, 2-amino-guanosine, LNA-guanosine, and 2-azido-guanosine.

A 5′-cap (cap0 or cap1) structure may be formed in chemical RNA synthesis, using capping enzymes, or in RNA in vitro transcription (co-transcriptional capping) using cap analogues.

The term “cap analogue” as used herein is intended to refer to a non-polymerizable di-nucleotide or tri-nucleotide that has cap functionality in that it facilitates translation or localization, and/or prevents degradation the RNA when incorporated at the 5′-end of the RNA. Non-polymerizable means that the cap analogue will be incorporated only at the 5′-terminus because it does not have a 5′ triphosphate and therefore cannot be extended in the 3′-direction by a template-dependent polymerase, (e.g. a DNA-dependent RNA polymerase). Examples of cap analogues include m7GpppG, m7GpppA, m7GpppC; unmethylated cap analogues (e.g. GpppG); dimethylated cap analogue (e.g. m2,7GpppG), trimethylated cap analogue (e.g. m2,2,7GpppG), dimethylated symmetrical cap analogues (e.g. m7Gpppm7G), or anti reverse cap analogues (e.g. ARCA; m7,2′OmeGpppG, m7,2′dGpppG, m7,3′OmeGpppG, m7,3′dGpppG and their tetraphosphate derivatives). Further cap analogues have been described previously (WO2008/016473, WO2008/157688, WO2009/149253, WO2011/015347, and WO2013/059475). Further suitable cap analogues in that context are described in WO2017/066793, WO2017/066781, WO2017/066791, WO2017/066789, WO2017/053297, WO2017/066782, WO2018/075827 and WO2017/066797 wherein the disclosures relating to cap analogues are incorporated herewith by reference.

In embodiments, a cap1 structure is generated using tri-nucleotide cap analogue as disclosed in WO2017/053297, WO2017/066793, WO2017/066781, WO2017/066791, WO2017/066789, WO2017/066782, WO2018/075827 and WO2017/066797. In particular, any cap analog derivable from the structure disclosed in claim 1-5 of WO2017/053297 may be suitably used to co-transcriptionally generate a cap1 structure. Further, any cap analog derivable from the structure defined in claim 1 or claim 21 of WO2018/075827 may be suitably used to co-transcriptionally generate a cap1 structure.

In preferred embodiments, the RNA of the pharmaceutical composition of the invention (e.g. component B) comprises a cap1 structure.

In preferred embodiments, the cap1 structure of the RNA is formed using co-transcriptional capping using tri-nucleotide cap analog m7G(5′)ppp(5′)(2′OMeA)pG or m7G(5′)ppp(5′)(2′OMeG)pG. A preferred cap1 analog in that context is m7G(5′)ppp(5′)(2′OMeA)pG.

In embodiments, about 70%, 75%, 80%, 85%, 90%, 95% of the RNA (species) of the pharmaceutical composition comprises a cap structure, preferably a cap1 structure, as determined using a capping assay. In preferred embodiments, less than about 20%, 15%, 10%, 5%, 4%, 3%, 2%, 1% of the RNA (species) does not comprises a cap structure as determined using a capping assay.

In preferred embodiments, at least 70%, 80%, or 90% of the RNA of the pharmaceutical composition comprise a cap1 structure.

For determining the presence/absence of a cap0 or a cap1 structure, a capping assays as described in published PCT application WO2015/101416, in particular, as described in claims 27 to 46 of published PCT application WO2015/101416 may be used. Other capping assays that may be used to determine the presence/absence of a cap0 or a cap1 structure of an RNA are described in published PCT application WO2020/127959.

In preferred embodiments, the RNA of the pharmaceutical composition comprises an m7G(5′)ppp(5′)(2′OMeA) cap structure. In such embodiments, the RNA comprises a 5-terminal m7G cap, and an additional methylation of the ribose of the adjacent nucleotide of m7GpppN, in that case, a 2′O methylated Adenosine. Preferably, about 70%, 75%, 80%, 85%, 90%, 95% of the RNA (species) comprises such a cap1 structure as determined using a capping assay. Preferably, about 95% of the RNA (species) comprises a cap1 structure in the correct orientation (and less that about 5% in reverse orientation) as determined using a capping assay.

In other preferred embodiments, the RNA of the pharmaceutical composition comprises an m7G(5′)ppp(5′)(2′OMeG) cap structure. In such embodiments, the RNA comprises a 5-terminal m7G cap, and an additional methylation of the ribose of the adjacent nucleotide, in that case, a 2′O methylated guanosine. Preferably, about 70%, 75%, 80%, 85%, 90%, 95% of the coding RNA (species) comprises such a cap1 structure as determined using a capping assay.

Accordingly, the first nucleotide of said RNA or mRNA sequence, that is, the nucleotide downstream of the m7G(5′)ppp structure, may be a 2′O methylated guanosine or a 2′O methylated adenosine.

In preferred embodiments, the RNA of the pharmaceutical composition of the invention (e.g. component B) comprises at least one poly(A) sequence, and/or at least one poly(C) sequence, and/or at least one histone stem-loop and/or at least one 5′-UTR and/or at least one 3′-UTR.

In preferred embodiments, the RNA of the pharmaceutical composition comprises at least one heterologous untranslated region (UTR).

The term “untranslated region” or “UTR” or “UTR element” are intended to refer to a part of an RNA typically located 5′ or 3′ of a coding sequence. An UTR is not translated into protein. An UTR typically comprises elements for controlling gene expression, also called regulatory elements. Such regulatory elements may be, e.g., ribosomal binding sites, miRNA binding sites, promotor elements etc.

UTRs may harbor regulatory sequence elements that determine RNA turnover, stability, and localization. Moreover, UTRs may harbor sequence elements that enhance translation. In medical application of and RNA, translation into at least one peptide or protein may be of paramount importance to therapeutic efficacy. Certain combinations of 3′-UTRs and/or 5′-UTRs may enhance the expression of operably linked coding sequences encoding peptides or proteins of the invention. RNA harboring said UTR combinations advantageously enable rapid and transient expression of peptides or proteins after administration to a subject.

In embodiments, the RNA comprises at least one 5′-UTR, preferably a heterologous 5′-UTR and/or at least one 3′-UTR, preferably a heterologous 3′-UTR.

Heterologous 5′-UTRs or 3′-UTRs may be derived from naturally occurring genes or may be synthetically engineered. In preferred embodiments, the RNA comprises at least one coding sequence as defined herein operably linked to at least one (heterologous) 3′-UTR and/or at least one (heterologous) 5′-UTR.

In preferred embodiments, the RNA of the pharmaceutical composition comprises at least one heterologous 3′-UTR.

The term “3′-untranslated region” or “3′-UTR” or “3′-UTR element” are intended to refer to a part of an RNA molecule located 3′ (i.e. downstream) of a coding sequence and which is not translated into protein. A 3′-UTR may be part of an RNA, located between a coding sequence and an optional terminal poly(A) sequence. A 3′-UTR typically comprises elements for controlling gene expression, also called regulatory elements. Such regulatory elements may be, e.g., ribosomal binding sites, miRNA binding sites etc. The 3′-UTR may be post-transcriptionally modified, e.g. by enzymatic or post-transcriptional addition of a Poly-A tail.

Preferably, the RNA comprises a 3′-UTR, which may be derivable from a gene that relates to an RNA with enhanced half-life (i.e. that provides a stable RNA).

In some embodiments, a 3′-UTR comprises one or more of a polyadenylation signal, a binding site for proteins that affect a nucleic acid stability of location in a cell, or one or more miRNA or binding sites for miRNAs.

In preferred embodiments, the RNA of the pharmaceutical composition comprises at least one heterologous 3′-UTR, wherein the at least one heterologous 3′-UTR comprises a nucleic acid sequence that is derived or that is selected from a 3′-UTR of a gene selected from PSMB3, ALB7, alpha-globin (referred to as “muag”), CASP1, COX6B1, GNAS, NDUFA1 and RPS9, or from a homolog, a fragment or variant of any one of these genes, preferably according to nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 253-268 of PCT/EP2021/052455 or WO2021156267 or a fragment or a variant of any of these. Particularly preferred nucleic acid sequences in that context can be derived from published PCT application WO2019/077001A1, in particular, claim 9 of WO2019/077001A1. The corresponding 3′-UTR sequences of claim 9 of WO2019/077001A1 are herewith incorporated by reference (e.g., SEQ ID NOs: 23-34 of WO2019/077001A1, or fragments or variants thereof).

In embodiments, the RNA of the pharmaceutical composition comprises a 3′-UTR derived from an alpha-globin gene. Said 3′-UTR derived from a alpha-globin gene (“muag”) may comprise or consist of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 267 or 268 of PCT/EP2021/052455 or WO2021156267 or a fragment or a variant thereof.

In preferred embodiments, the RNA of the pharmaceutical composition comprises a 3′-UTR derived from a PSMB3 gene. Said 3′-UTR derived from a PSMB3 gene may comprise or consist of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 253 or 254 of PCT/EP2021/052455 or WO2021156267 or a fragment or a variant thereof.

In other embodiments, the RNA of the pharmaceutical composition may comprise a 3′-UTR as described in WO2016/107877, the disclosure of WO2016/107877 relating to 3′-UTR sequences herewith incorporated by reference. Suitable 3′-UTRs are SEQ ID NOs: 1-24 and SEQ ID NOs: 49-318 of WO2016/107877, or fragments or variants of these sequences. In other embodiments, the RNA may comprise a 3′-UTR as described in WO2017/036580, the disclosure of WO2017/036580 relating to 3′-UTR sequences herewith incorporated by reference. Suitable 3′-UTRs are SEQ ID NOs: 152-204 of WO2017/036580, or fragments or variants of these sequences. In other embodiments, the RNA may comprise a 3′-UTR as described in WO2016/022914, the disclosure of WO2016/022914 relating to 3′-UTR sequences herewith incorporated by reference. Particularly preferred 3′-UTRs are nucleic acid sequences according to SEQ ID NOs: 20-36 of WO2016/022914, or fragments or variants of these sequences.

In preferred embodiments, the RNA of the pharmaceutical composition comprises at least one heterologous 5′-UTR.

The terms “5′-untranslated region” or “5′-UTR” or “5′-UTR element” are intended to refer to a part of an RNA molecule located 5′ (i.e. “upstream”) of a coding sequence and which is not translated into protein. A 5′-UTR may be part of an RNA located 5′ of the coding sequence. Typically, a 5′-UTR starts with the transcriptional start site and ends before the start codon of the coding sequence. A 5′-UTR may comprise elements for controlling gene expression, also called regulatory elements. Such regulatory elements may be, e.g., ribosomal binding sites, miRNA binding sites etc. The 5′-UTR may be post-transcriptionally modified, e.g. by enzymatic or post-transcriptional addition of a 5′-cap structure.

Preferably, the RNA of the pharmaceutical composition comprises a 5′-UTR, which may be derivable from a gene that relates to an RNA with enhanced half-life (i.e. that provides a stable RNA).

In some embodiments, a 5′-UTR comprises one or more of a binding site for proteins that affect an RNA stability or RNA location in a cell, or one or more miRNA or binding sites for miRNAs.

In preferred embodiments, the RNA of the pharmaceutical composition comprises at least one heterologous 5′-UTR, wherein the at least one heterologous 5′-UTR comprises a nucleic acid sequence is derived or selected from a 5′-UTR of gene selected from HSD17B4, RPL32, ASAH1, ATP5A1, MP68, NDUFA4, NOSIP, RPL31, SLC7A3, TUBB4B, and UBQLN2, or from a homolog, a fragment or variant of any one of these genes according to nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 231-252 of PCT/EP2021/052455 or WO2021156267 or a fragment or a variant of any of these. Particularly preferred nucleic acid sequences in that context can be selected from published PCT application WO2019/077001A1, in particular, claim 9 of WO2019/077001A1. The corresponding 5′-UTR sequences of claim 9 of WO2019/077001A1 are herewith incorporated by reference (e.g., SEQ ID NOs: 1-20 of WO2019/077001A1, or fragments or variants thereof).

In preferred embodiments, the RNA of the pharmaceutical composition comprises a 5′-UTR derived or selected from a HSD17B4 gene, wherein said 5′-UTR derived from a HSD17B4 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 231 or 232 of PCT/EP2021/052455 or WO2021156267 or a fragment or a variant thereof.

In other embodiments, the RNA of the pharmaceutical composition may comprise a 5′-UTR as described in WO2013/143700, the disclosure of WO2013/143700 relating to 5′-UTR sequences herewith incorporated by reference. Particularly preferred 5′-UTRs are nucleic acid sequences derived from SEQ ID NOs: 1-1363, SEQ ID NO: 1395, SEQ ID NO: 1421 and SEQ ID NO: 1422 of WO2013/143700, or fragments or variants of these sequences. In other embodiments, the RNA may comprises a 5′-UTR as described in WO2016/107877, the disclosure of WO2016/107877 relating to 5′-UTR sequences herewith incorporated by reference. Particularly preferred 5′-UTRs are nucleic acid sequences according to SEQ ID NOs: 25-30 and SEQ ID NOs: 319-382 of WO2016/107877, or fragments or variants of these sequences. In other embodiments, the nucleic acid comprises a 5′-UTR as described in WO2017/036580, the disclosure of WO2017/036580 relating to 5′-UTR sequences herewith incorporated by reference. Particularly preferred 5′-UTRs are nucleic acid sequences according to SEQ ID NOs: 1-151 of WO2017/036580, or fragments or variants of these sequences. In other embodiments, the RNA may comprise a 5′-UTR as described in WO2016/022914, the disclosure of WO2016/022914 relating to 5′-UTR sequences herewith incorporated by reference. Particularly preferred 5′-UTRs are nucleic acid sequences according to SEQ ID NOs: 3-19 of WO2016/022914, or fragments or variants of these sequences.

In various embodiments, the RNA of the pharmaceutical composition may comprise a 5-terminal sequence element according to SEQ ID NOs: 176 or 177 of PCT/EP2021/052455 or WO2021156267, or a fragment or variant thereof. Such a 5-terminal sequence element comprises e.g. a binding site for T7 RNA polymerase. Further, the first nucleotide of said 5′-terminal start sequence may preferably comprise a 2′O methylation, e.g. 2′O methylated guanosine or a 2′O methylated adenosine (which is an element of a Cap1 structure).

In particularly preferred embodiments, the RNA of the pharmaceutical composition comprises at least one coding sequence as defined wherein said coding sequence is operably linked to a HSD17B4 5′-UTR and a PSMB3 3′-UTR (HSD17B4/PSMB3).

In particularly preferred embodiments, the RNA of the pharmaceutical composition comprises at least one coding sequence as defined herein, wherein said coding sequence is operably linked to an alpha-globin (“muag”) 3′-UTR.

In various embodiments, the RNA of the pharmaceutical composition comprises at least one poly(N) sequence, e.g. at least one poly(A) sequence, at least one poly(U) sequence, at least one poly(C) sequence, or combinations thereof.

In preferred embodiments, the RNA of the pharmaceutical composition comprises at least one poly(A) sequence.

The terms “poly(A) sequence”, “poly(A) tail” or “3′-poly(A) tail” as used herein will be recognized and understood by the person of ordinary skill in the art, and are e.g. intended to be a sequence of adenosine nucleotides, typically located at the 3′-end of an RNA of up to about 1000 adenosine nucleotides. Preferably, said poly(A) sequence is essentially homopolymeric, e.g. a poly(A) sequence of e.g. 100 adenosine nucleotides has essentially the length of 100 nucleotides.

In other embodiments, the poly(A) sequence may be interrupted by at least one nucleotide different from an adenosine nucleotide, e.g. a poly(A) sequence of e.g. 100 adenosine nucleotides may have a length of more than 100 nucleotides (comprising 100 adenosine nucleotides and in addition said at least one nucleotide—or a stretch of nucleotides—different from an adenosine nucleotide). For example, the poly(A) sequence may comprise about 100 A nucleotides being interrupted by at least one nucleotide different from A (e.g. a linker (L), typically about 2 to 20 nucleotides in length), e.g. A30-L-A70 or A70-L-A30.

The poly(A) sequence may comprise about 10 to about 500 adenosine nucleotides, about 10 to about 200 adenosine nucleotides, about 40 to about 200 adenosine nucleotides, or about 40 to about 150 adenosine nucleotides. Suitably, the length of the poly(A) sequence may be at least about or even more than about 10, 50, 64, 75, 100, 200, 300, 400, or 500 adenosine nucleotides. In preferred embodiments, the at least one nucleic acid comprises at least one poly(A) sequence comprising about 30 to about 200 adenosine nucleotides. In particularly preferred embodiments, the poly(A) sequence comprises about 64 adenosine nucleotides (A64). In other particularly preferred embodiments, the poly(A) sequence comprises about 100 adenosine nucleotides (A100). In other embodiments, the poly(A) sequence comprises about 150 adenosine nucleotides.

The poly(A) sequence as defined herein may be located directly at the 3′ terminus of the at least one nucleic acid, preferably directly located at the 3′ terminus of an RNA. In such embodiments, the 3-terminal nucleotide (that is the last 3′-terminal nucleotide in the polynucleotide chain) is the 3′-terminal A nucleotide of the at least one poly(A) sequence. The term “directly located at the 3′ terminus” has to be understood as being located exactly at the 3′ terminus—in other words, the 3′ terminus of the nucleic acid consists of a poly(A) sequence terminating with an A nucleotide.

In embodiments, the RNA of the pharmaceutical composition may comprise a poly(A) sequence obtained by enzymatic polyadenylation, wherein the majority of nucleic acid molecules comprise about 100 (+/−20) to about 500 (+/−50), preferably about 250 (+/−20) adenosine nucleotides.

In embodiments, the RNA of the pharmaceutical composition comprises a poly(A) sequence derived from a template DNA and additionally comprises at least one poly(A) sequence generated by enzymatic polyadenylation, e.g. as described in WO2016/091391.

In embodiments, the RNA of the pharmaceutical composition comprises at least one polyadenylation signal.

In embodiments, the RNA of the pharmaceutical composition comprises at least one poly(C) sequence.

The term “poly(C) sequence” as used herein is intended to be a sequence of cytosine nucleotides of up to about 200 cytosine nucleotides. In preferred embodiments, the poly(C) sequence comprises about 10 to about 200 cytosine nucleotides, about 10 to about 100 cytosine nucleotides, about 20 to about 70 cytosine nucleotides, about 20 to about 60 cytosine nucleotides, or about 10 to about 40 cytosine nucleotides. In a particularly preferred embodiment, the poly(C) sequence comprises about 30 cytosine nucleotides.

In embodiments, the RNA of the pharmaceutical composition comprises at least one histone stem-loop (hSL) or histone stem loop structure.

The term “histone stem-loop” (abbreviated as “hSL” in e.g. the sequence listing) is intended to refer to nucleic acid sequences that form a stem-loop secondary structure predominantly found in histone mRNAs.

Histone stem-loop sequences/structures may suitably be selected from histone stem-loop sequences as disclosed in WO2012/019780, the disclosure relating to histone stem-loop sequences/histone stem-loop structures incorporated herewith by reference. A histone stem-loop sequence may preferably be derived from formulae (I) or (II) of WO2012/019780. According to a further preferred embodiment, the RNA comprises at least one histone stem-loop sequence derived from at least one of the specific formulae (Ia) or (Ila) of the patent application WO2012/019780.

In preferred embodiments, the RNA of the pharmaceutical composition comprises at least one histone stem-loop, wherein said histone stem-loop (hSL) comprises or consists a nucleic acid sequence identical or at least 70%, 80%, 90%, 91%, 92%, 93%, 94%, 95%, 96%, 97%, 98%, or 99% identical to SEQ ID NOs: 178 or 179 of PCT/EP2021/052455 or WO2021156267, or fragments or variants thereof.

In embodiments, the RNA of the pharmaceutical composition comprises a 3-terminal sequence element. Said 3′-terminal sequence element comprises a poly(A) sequence and a histone-stem-loop sequence. Accordingly, the RNA comprises at least one 3′-terminal sequence element comprising or consisting of a nucleic acid sequence being identical or at least 70%, 80%, 90%, 91%, 92%, 93%, 94%, 95%, 96%, 97%, 98%, or 99% identical to SEQ ID NOs: 182 to 230 of PCT/EP2021/052455 or WO2021156267, or a fragment or variant thereof.

In embodiments, the RNA of the pharmaceutical composition may be monocistronic, bicistronic, or multicistronic.

The term “monocistronic” will be recognized and understood by the person of ordinary skill in the art, and is e.g. intended to refer to an RNA that comprises only one coding sequence. The terms “bicistronic”, or “multicistronic” as used herein will be recognized and understood by the person of ordinary skill in the art, and are e.g. intended to refer to an RNA that may comprise two (bicistronic) or more (multicistronic) coding sequences.

In preferred embodiments, the RNA of the pharmaceutical composition is monocistronic.

In embodiments, the RNA of the pharmaceutical composition may be bicistronic or multicistronic and comprises at least two coding sequences. Accordingly, the coding sequences in a bicistronic or multicistronic RNA suitably encodes distinct proteins or peptides as defined herein or fragments or variants thereof. Preferably, the coding sequences in said bicistronic or multicistronic constructs may be separated by at least one IRES (internal ribosomal entry site) sequence. In that context, suitable IRES sequences may be selected from the list of nucleic acid sequences according to SEQ ID NOs: 1566-1662 of the patent application WO2017/081082, or fragments or variants of these sequences. In this context, the disclosure of WO2017/081082 relating to IRES sequences is herewith incorporated by reference.

In embodiments, the A/U (A/T) content in the environment of the ribosome binding site of the RNA may be increased compared to the A/U (A/T) content in the environment of the ribosome binding site of its respective wild type nucleic acid. This modification (an increased A/U (A/T) content around the ribosome binding site) increases the efficiency of ribosome binding to the RNA. An effective binding of the ribosomes to the ribosome binding site in turn has the effect of an efficient translation the RNA. Accordingly, in a particularly preferred embodiment, the RNA of the composition comprises a ribosome binding site, also referred to as “Kozak sequence” identical to or at least 80%, 85%, 90%, 95% identical to any one of the sequences SEQ ID NOs: 180 or 181 of PCT/EP2021/052455 or WO2021156267, or fragments or variants thereof.

In particularly preferred embodiments, the RNA of the pharmaceutical composition is an in vitro transcribed RNA.

In various embodiments the RNA comprises, preferably in 5′- to 3′-direction, the following elements:
A) 5′-cap structure, preferably as specified herein;
B) 5′-terminal start element, preferably as specified herein;
C) optionally, a 5′-UTR, preferably as specified herein;
D) a ribosome binding site, preferably as specified herein;
E) at least one coding sequence, preferably as specified herein;
F) 3′-UTR, preferably as specified herein;
G) optionally, poly(A) sequence, preferably as specified herein;
H) optionally, poly(C) sequence, preferably as specified herein;
I) optionally, histone stem-loop preferably as specified herein;
J) optionally, 3′-terminal sequence element, preferably as specified herein.

In particularly preferred embodiments the RNA, comprises the following elements in 5′- to 3′-direction:
A) cap1 structure as defined herein;
B) 5′-terminal start element, preferably as specified herein;
C) coding sequence as specified herein, preferably according to SEQ ID NOs: 3 or 4;
D) 3′-UTR derived from a 3′-UTR of a muag gene as defined herein, preferably according to SEQ ID NO: 267 or 268 of PCT/EP2021/052455 or WO2021156267;
E) poly(A) sequence comprising about 64 A nucleotides.
F) poly(C) sequence comprising about 10 to about 100 cytosines;
G) histone stem-loop selected from SEQ ID NOs: 178 or 179 of PCT/EP2021/052455 or WO2021156267;

In particularly preferred embodiments the at least one nucleic acid, preferably the mRNA, comprises the following elements in 5′- to 3′-direction:
A) cap1 structure as defined herein;
B) 5-terminal start element, preferably as specified herein;
C) 5′-UTR derived from a HSD17B4 gene as defined herein, preferably according to SEQ ID NO: 231 or 232 of PCT/EP2021/052455 or WO2021156267;
D) coding sequence selected as specified herein, preferably according to SEQ ID NOs: 3 or 4;
E) 3′-UTR derived from a 3′-UTR of a PSMB3 gene as defined herein, preferably according to SEQ ID NO: 253 or 254 of PCT/EP2021/052455 or WO2021156267;
F) optionally, a histone stem-loop selected from SEQ ID NOs: 178 or 179 of PCT/EP2021/052455 or WO2021156267;
G) poly(A) sequence comprising about 100 A nucleotides, preferably representing the 3′ terminus.

In the context of the invention, the RNA of the pharmaceutical composition may provide at least one coding sequence encoding a peptide or protein that is translated into a (functional) peptide or protein after administration (e.g. after administration to a subject, e.g. a human subject).

In preferred embodiments, the coding sequence of the RNA encodes at least one peptide or protein, wherein said at least one peptide or protein is selected or derived from a therapeutic peptide or protein.

In preferred embodiments, the RNA of the pharmaceutical composition of the invention (e.g. component B) comprises at least one coding sequence encoding at least one peptide or protein suitable for use in treatment or prevention of a disease, disorder or condition.

In various embodiments, the length of the encoded peptide or protein, e.g. the therapeutic peptide or protein, may be at least or greater than about 20, 50, 100, 150, 200, 300, 400, 500, 600, 700, 800, 900, 1000, or 1500 amino acids.

In preferred embodiments, the at least one (therapeutic) peptide or protein is selected or is derived from an antibody, an intrabody, a receptor, a receptor agonist, a receptor antagonist, a binding protein, a CRISPR-associated endonuclease, a chaperone, a transporter protein, an ion channel, a membrane protein, a secreted protein, a transcription factor, an enzyme, a peptide or protein hormone, a growth factor, a structural protein, a cytoplasmic protein, a cytoskeletal protein, a viral antigen, a bacterial antigen, a protozoan antigen, an allergen, a tumor antigen, or fragments, variants, or combinations of any of these.

In embodiments, the peptide or protein is selected from an antigen or epitope of a pathogen selected or derived from List 1 provided below.

### List 1: Suitable Pathogens of the Invention

Acinetobacter baumannii, Anaplasma genus, Anaplasma phagocytophilum, Ancylostoma braziliense, Ancylostoma duodenale, Arcanobacterium haemolyticum, Ascaris lumbricoides, Aspergillus genus, Astroviridae, Babesia genus, Bacillus anthracis, Bacillus cereus, Bartonella henselae, BK virus, Blastocystis hominis, Blastomyces dermatitidis, Bordetella pertussis, Borrelia burgdorferi, Borrelia genus, Borrelia spp, Brucella genus, Brugia malayi, Bunyaviridae family, Burkholderia cepacia and other Burkholderia species, Burkholderia mallei, Burkholderia pseudomallei, Caliciviridae family, Campylobacter genus, Candida albicans, Candida spp, Chlamydia trachomatis, Chlamydophila pneumoniae, Chlamydophila psittaci, CJD prion, Clonorchis sinensis, Clostridium botulinum, Clostridium difficile, Clostridium perfringens, Clostridium perfringens, Clostridium spp, Clostridium tetani, Coccidioides spp, coronaviruses, Corynebacterium diphtheriae, Coxiella burnetii, Crimean-Congo hemorrhagic fever virus, Cryptococcus neoformans, Cryptosporidium genus, Cytomegalovirus (CMV), Dengue viruses (DEN-1, DEN-2, DEN-3 and DEN-4), Dientamoeba fragilis, Ebolavirus (EBOV), Echinococcus genus, Ehrlichia chaffeensis, Ehrlichia ewingii, Ehrlichia genus, Entamoeba histolytica, Enterococcus genus, Enterovirus genus, Enteroviruses, mainly Coxsackie A virus and Enterovirus 71 (EV71), Epidermophyton spp, Epstein-Barr Virus (EBV), Escherichia coli 0157:H7, 0111 and 0104:H4, Fasciola hepatica and Fasciola gigantica, FFI prion, Filarioidea superfamily, Flaviviruses, Francisella tularensis, Fusobacterium genus, Geotrichum candidum, Giardia intestinalis, Gnathostoma spp, GSS prion, Guanarito virus, Haemophilus ducreyi, Haemophilus influenzae, Helicobacter pylori, Henipavirus (Hendra virus Nipah virus), Hepatitis A Virus, Hepatitis B Virus (HBV), Hepatitis C Virus (HCV), Hepatitis D Virus, Hepatitis E Virus, Herpes simplex virus 1 and 2 (HSV-1 and HSV-2), Histoplasma capsulatum, HIV (Human immunodeficiency virus), Hortaea werneckii, Human bocavirus (HBoV), Human herpesvirus 6 (HHV-6) and Human herpesvirus 7 (HHV-7), Human metapneumovirus (hMPV), Human papillomavirus (HPV), Human parainfluenza viruses (HPIV), Japanese encephalitis virus, JC virus, Junin virus, Kingella kingae, Klebsiella granulomatis, Kuru prion, Lassa virus, Legionella pneumophila, Leishmania genus, Leptospira genus, Listeria monocytogenes, Lymphocytic choriomeningitis virus (LCMV), Machupo virus, Malassezia spp, Marburg virus, Measles virus, Metagonimus yokagawai, Microsporidia phylum, Molluscum contagiosum virus (MCV), Mumps virus, Mycobacterium leprae and Mycobacterium lepromatosis, Mycobacterium tuberculosis, Mycobacterium ulcerans, Mycoplasma pneumoniae, Naegleria fowleri, Necator americanus, Neisseria gonorrhoeae, Neisseria meningitidis, Nocardia asteroides, Nocardia spp, Onchocerca volvulus, Orientia tsutsugamushi, Orthomyxoviridae family (Influenza), Paracoccidioides brasiliensis, Paragonimus spp, Paragonimus westermani, Parvovirus B19, Pasteurella genus, Plasmodium genus, Pneumocystis jirovecii, Poliovirus, Rabies virus, Respiratory syncytial virus (RSV), Rhinovirus, rhinoviruses, Rickettsia akari, Rickettsia genus, Rickettsia prowazekii, Rickettsia rickettsii, Rickettsia typhi, Rift Valley fever virus, Rotavirus, Rubella virus, Sabia virus, Salmonella genus, Sarcoptes scabiei, SARS coronavirus, SARS-CoV-2 coronavirus, Schistosoma genus, Shigella genus, Sin Nombre virus, Hantavirus, Sporothrix schenckii, Staphylococcus genus, Staphylococcus genus, Streptococcus agalactiae, Streptococcus pneumoniae, Streptococcus pyogenes, Strongyloides stercoralis, Taenia genus, Taenia solium, Tick-borne encephalitis virus (TBEV), Toxocara canis or Toxocara cati, Toxoplasma gondii, Treponema pallidum, Trichinella spiralis, Trichomonas vaginalis, Trichophyton spp, Trichuris trichiura, Trypanosoma brucei, Trypanosoma cruzi, Ureaplasma urealyticum, Varicella zoster virus (VZV), Varicella zoster virus (VZV), Variola major or Variola minor, vCJD prion, Venezuelan equine encephalitis virus, Vibrio cholerae, West Nile virus, Western equine encephalitis virus, Wuchereria bancrofti, Yellow fever virus, Yersinia enterocolitica, Yersinia pestis, and Yersinia pseudotuberculosis.

In particularly preferred embodiments, the peptide or protein (encoded by the RNA of the pharmaceutical composition) is selected from an antigen or epitope of a pathogen, preferably selected or derived from a Coronavirus, e.g. SARS-CoV-2, or a fragment or variant of any of these.

According to preferred embodiments, the RNA of the pharmaceutical composition of the invention (e.g. component B) comprises a coding sequence encoding at least one antigen or epitope selected or derived from a Coronavirus, preferably SARS-CoV-2.

In specific embodiments, the RNA of the pharmaceutical composition of the invention (e.g. component B) comprises a coding sequence encoding at least one antigen or epitope selected or derived from a Coronavirus, preferably SARS-CoV-2 spike protein (S). Preferably, the spike protein (S) is a pre-fusion stabilized spike protein comprising at least one pre-fusion stabilizing mutation. Suitably, the at least one pre-fusion stabilizing mutation comprises the following amino acid substitutions: K986P and V987P.

In specific embodiments, the RNA of the pharmaceutical composition of the invention (e.g. component B) comprises a coding sequence encoding at least one antigen or epitope selected or derived from a Coronavirus, preferably SARS-CoV-2 spike protein (S), wherein the spike protein (S) is a pre-fusion stabilized spike protein comprising at least one pre-fusion stabilizing mutation, wherein the at least one antigenic peptide or protein comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 1 or 2 or an immunogenic fragment or immunogenic variant of any of these.

In specific embodiments, the RNA of the pharmaceutical composition of the invention (e.g. component B) comprises a coding sequence encoding an S protein comprising a pre-fusion stabilizing K986P and V987P mutation, wherein the coding sequence comprises or consists of a G/C optimized coding sequence comprising a nucleic acid sequence being identical to SEQ ID NOs: 3 or 4 or a fragment or variant thereof. Preferably, the coding sequences do not comprise chemically modified nucleotides.

In a preferred embodiment, the RNA encoding the antigen or epitope selected or derived from a SARS-CoV-2 virus comprises or consists of a nucleic acid sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 5 to 10 or a fragment or variant of that sequence. Preferably, the RNA sequence does not comprise chemically modified nucleotides. Preferably, the RNA comprises a 5′ Cap1 structure.

In embodiments, pharmaceutical composition of the invention (e.g. component B) comprises a plurality or at least more than one of the RNA species as defined herein. Preferably, the pharmaceutical composition as defined herein may comprise 2, 3, 4, 5, 6, 7, 8, 9, or 10 different nucleic acids each as defined herein.

In embodiments, the pharmaceutical composition comprises at least 2, 3, 4, 5, 6, 7, 8, 9, 10 or even more different RNA species as defined herein, each encoding at least one peptide or protein derived from a genetically different pathogen, in particular coronavirus (e.g. a different coronavirus isolate), or a fragment or variant thereof. The terms “different pathogen” or “different coronavirus” have to be understood as the difference between at least two respective pathogens or coronavirus (e.g. a different coronavirus isolates), wherein the difference is manifested on the genome of the respective different coronavirus. Particularly, said (genetically) different coronavirus may express at least one different protein, peptide or polyprotein, wherein the at least one different protein, peptide or polyprotein differs in at least one amino acid.

In preferred embodiments, the RNA of the pharmaceutical composition is complexed or associated with at least one further compound to obtain a formulated composition. A formulation in that context may have the function of a transfection agent. A formulation in that context may also have the function of protecting the nucleic acid from degradation.

In various embodiment, the RNA of the pharmaceutical composition is formulated in a pharmaceutically acceptable carrier or excipient.

In preferred embodiments, the RNA of the pharmaceutical composition is formulated in at least one cationic or polycationic compound, e.g. cationic or polycationic peptides, proteins, lipids, polysaccharides, and/or polymers.

Accordingly, the RNA of the pharmaceutical composition may be complexed or associated with, or at least partially complexed or partially associated with one or more cationic or polycationic compound, preferably cationic or polycationic polymer, cationic or polycationic polysaccharide, cationic or polycationic lipid, cationic or polycationic protein, cationic or polycationic peptide, or any combinations thereof.

In preferred embodiments, the RNA is formulated in lipid-based carriers. Accordingly, in particularly preferred embodiments, the pharmaceutical composition of the invention, e.g. the pharmaceutical composition of the kit or kit of parts of the first aspect, or the syringe of the second aspect, comprises RNA formulated in lipid-based carriers.

In the context of the invention, the term “lipid-based carriers” encompass lipid based delivery systems for RNA that comprise a lipid component. A lipid-based carrier may additionally comprise other components suitable for encapsulating/incorporating/complexing an RNA including a cationic or polycationic polymer, a cationic or polycationic polysaccharide, a cationic or polycationic protein, a cationic or polycationic peptide, or any combinations thereof.

In the context of the invention, a typical “lipid-based carrier” is selected from liposomes, lipid nanoparticles (LNPs), lipoplexes, and/or nanoliposomes. The RNA of the pharmaceutical composition may completely or partially incorporated or encapsulated in a lipid-based carrier, wherein the RNA may be located in the interior space of the lipid-based carrier, within the lipid layer/membrane of the lipid-based carrier, or associated with the exterior surface of the lipid-based carrier. The incorporation of an RNA into lipid-based carriers is also referred to as “encapsulation”. A “lipid-based carrier” is not restricted to any particular morphology, and include any morphology generated when e.g. an aggregation reducing lipid and at least one further lipid are combined, e.g. in an aqueous environment in the presence of an RNA. For example, an LNP, a liposome, a lipid complex, a lipoplex and the like are within the scope of the term “lipid-based carrier”. Lipid-based carriers can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 nm and 500 nm in diameter. Liposomes, a specific type of lipid-based carrier, are characterized as microscopic vesicles having an interior aqua space sequestered from an outer medium by a membrane of one or more bilayers. In a liposome, the RNA is typically located in the interior aqueous space enveloped by some or the entire lipid portion of the liposome. Bilayer membranes of liposomes are typically formed by amphiphilic molecules, such as lipids of synthetic or natural origin that comprise spatially separated hydrophilic and hydrophobic domains. Lipid nanoparticles (LNPs), a specific type of lipid-based carrier, are characterized as microscopic lipid particles having a solid core or partially solid core. Typically, an LNP does not comprise an interior aqua space sequestered from an outer medium by a bilayer. In an LNP, the RNA may be encapsulated or incorporated in the lipid portion of the LNP enveloped by some or the entire lipid portion of the LNP. An LNP may comprise any lipid capable of forming a particle to which the RNA may be attached, or in which the RNA may be encapsulated.

In preferred embodiments, the RNA is encapsulated in the lipid-based carriers of the pharmaceutical composition of the invention (e.g. component B).

The term “encapsulated”, e.g. incorporated, complexed, encapsulated, partially encapsulated, associated, partially associated, refers to the essentially stable combination of RNA with one or more lipids into lipid-based carriers (e.g. larger complexes or assemblies) without covalent binding of the RNA. The lipid-based carriers—encapsulated RNA may be completely or partially located in the interior of the lipid-based carrier (e.g. the lipid portion and/or an interior space) and/or within the lipid layer/membrane of the lipid-based carriers. The encapsulation of an RNA into lipid-based carriers is also referred to herein as “incorporation” as the RNA is preferably contained within the interior of the lipid-based carriers.

Without wishing to be bound to theory, the purpose of incorporating or encapsulating RNA into lipid-based carriers may be to protect the RNA from an environment which may contain enzymes, chemicals, or conditions that degrade the RNA.

Moreover, incorporating RNA into lipid-based carriers may promote the uptake of the RNA, and hence, may enhance the therapeutic effect of the RNA when administered to a cell or a subject.

In various embodiments, the pharmaceutical composition comprises a certain concentration of lipid (or the lipid-based carriers encapsulating the RNA).

In embodiments, the concentration of lipid (or lipid-based carriers) in the pharmaceutical composition is in a range of about 2.5 μg/ml to about 250 mg/ml, 2.5 μg/ml to about 125 mg/ml, 2.5 μg/ml to about 50 mg/ml, 2.5 μg/ml to about 25 mg/ml, or 2.5 μg/ml to about 12.5 mg/ml.

In preferred embodiments, the concentration of lipid (or lipid-based carriers) in the pharmaceutical composition of the invention (e.g. component B) is in a range of about 2.5 μg/ml to about 12.5 mg/ml, more preferably in a range of about 25 μg/ml to about 5 mg/ml.

In embodiments, the concentration of lipid (or lipid-based carriers) in the pharmaceutical composition of the invention (e.g. component B) is for example about 25 μg/ml, about 50 μg/ml, about 75 μg/ml, about 100 μg/ml, about 125 μg/ml, about 250 μg/ml, about 500 μg/ml, about 750 μg/ml, about 1000 μg/ml, about 1250 μg/ml.

In embodiments, the concentration of lipid (or lipid-based carriers) in the liquid composition is lower than 5 mg/ml, preferably lower than 2.5 mg/ml, more preferably lower than 1.25 mg/ml.

In that context, “the concentration of lipid (or lipid-based carriers) in the liquid composition” relates to the total concentration of lipid (or lipid-based carriers) in the composition, and does not include any lipid or lipid-like contamination derived from storage in the syringe, e.g. silicone oil derived lipids or lipid-like compounds or lubricant oil derived lipids or lipid-like compounds.

In embodiments, the weight to weight (wt/wt) ratio of lipid to the RNA (in the lipid-based carriers) is from about 10:1 to about 60:1. In preferred embodiments, the weight to weight (wt/wt) ratio of lipid to the RNA (in the lipid-based carriers) is from about 20:1 to about 30:1. In embodiments, the weight to weight (wt/wt) ratio of lipid to the RNA (in the lipid-based carriers) is for example about 20:1, about 21:1, about 22:1, about 23:1, about 24:1, about 25:1, about 26:1, about 27:1, about 28:1, about 29:1, or about 30:1. In particularly preferred embodiments, the wt/wt ratio of lipid to the RNA (in the lipid-based carriers) is about 25:1.

In embodiments, the RNA to total lipid ratio in the lipid based carriers is less than about 0.1 w/w, preferably less than about 0.06 w/w. In preferred embodiments, the RNA to total lipid ratio in the lipid based carriers is between about 0.03 w/w and 0.05 w/w. In particularly preferred embodiments, the RNA to total lipid ratio in the lipid based carriers is between about 0.04 w/w.

The amount of lipid comprised in the lipid-based carriers may be selected taking the amount of the RNA cargo into account. In one embodiment, these amounts are selected such as to result in an N/P ratio of the lipid-based carriers encapsulating the RNA in the range of about 0.1 to about 20. The N/P ratio is defined as the mole ratio of the nitrogen atoms (“N”) of the basic nitrogen-containing groups of the lipid to the phosphate groups (“P”) of the RNA which is used as cargo. The N/P ratio may be calculated on the basis that, for example, 1 ug RNA typically contains about 3 nmol phosphate residues, provided that the RNA exhibits a statistical distribution of bases. The “N”-value of the lipid or lipidoid may be calculated on the basis of its molecular weight and the relative content of permanently cationic and—if present—cationisable groups.

In embodiments, the N/P ratio of the lipid-based carriers to the RNA is in a range from about 1 to about 10, preferably in a range from about 1 to about 7, more preferably in a range from about 5 to about 7, e.g. about 5.5, about 5.6, about 5.7, about 5.8, about 5.9, about 6, about 6.1, about 6.2, bout 6.3, about 6.4, about 6.5. In preferred embodiments, the N/P ratio of the lipid-based carriers to the RNA is about 6.

In various embodiments, the lipid-based carriers are monodisperse, meaning that the lipid-based carriers comprised in the composition have a uniform size. Typically, the distribution of size populations within a composition is expressed by the polydispersity index (PDI) value.

The term “polydispersity index” (PDI) is used herein as a measure of the size distribution of an ensemble of particles, e.g., lipid-based carriers. The polydispersity index is calculated based on dynamic light scattering measurements by the so-called cumulant analysis. Typically, the PDI is determined by dynamic light scattering at an angle of 90° or 173°, typically measured at a temperature of 25° C. PDI is basically a representation of the distribution of size populations within a given sample. The numerical value of PDI ranges from 0.0 (for a perfectly uniform sample with respect to the particle size) to 1.0 (for a highly polydisperse sample with multiple particle size populations).

In embodiments, the lipid-based carriers in the pharmaceutical composition of the invention (e.g. component B) have as a polydispersity index (PDI) value ranging from about 0.50 to about 0.00. In embodiments, the lipid-based carriers encapsulating the RNA have a polydispersity index (PDI) value of less than about 0.3, preferably of less than about 0.2, more preferably of less than about 0.15, most preferably of less than about 0.1.

In various embodiments, the pharmaceutical composition comprises lipid-based carriers that have a defined size (particle size, homogeneous size distribution).

The size of the lipid-based carriers of the pharmaceutical composition of the invention (e.g. component B) is typically described herein as Z-average size. The terms “average diameter”, “mean diameter”, “diameter” or “size” for particles (e.g. lipid-based carrier) are used synonymously with the value of the Z-average. The term “Z-average size” refers to the mean diameter of particles as measured by dynamic light scattering (DLS) with data analysis using the so-called cumulant algorithm, which provides as results the so-called Z-average with the dimension of a length, and the polydispersity index (PI), which is dimensionless (Koppel, D., J. Chem. Phys. 57, 1972, pp 4814-4820, ISO 13321).

The term “dynamic light scattering” or “DLS” refers to a method for analyzing particles in a liquid, wherein the liquid is typically illuminated with a monochromatic light source and wherein the light scattered by particles in the liquid is detected.

Due to Brownian motion, smaller particles typically result in time-dependent scattering intensity fluctuations that are distinct from those observed for larger particles. DLS can thus be used to measure particle sizes in a liquid. Suitable DLS protocols are known in the art. DLS instruments are commercially available (such as the Zetasizer Nano Series, Malvern Instruments, Worcestershire, UK). DLS instruments employ either a detector at 90° (e.g., DynaPro® NanoStar® from Wyatt Technology or Zetasizer Nano S90® from Malvern Instruments) or a backscatter detection system at 173° (e.g., Zetasizer Nano S® from Malvern Instruments) and at 158° (DynaPro Plate Reader® from Malvern Instruments) close to the incident light of 180°. Typically, DLS measurements are performed at a temperature of about 25° C. DLS is also used in the context of the present invention to determine the polydispersity index (PDI) and/or the main peak diameter of the lipid-based carriers incorporating RNA.

In various embodiments, the lipid-based carriers of the pharmaceutical composition of the invention (e.g. component B) have a Z-average size ranging from about 50 nm to about 200 nm, from about 50 nm to about 190 nm, from about 50 nm to about 180 nm, from about 50 nm to about 170 nm, from about 50 nm to about 160 nm, 50 nm to about 150 nm, 50 nm to about 140 nm, 50 nm to about 130 nm, 50 nm to about 120 nm, 50 nm to about 110 nm, 50 nm to about 100 nm, 50 nm to about 90 nm, 50 nm to about 80 nm, 50 nm to about 70 nm, 50 nm to about 60 nm, 60 nm to about 200 nm, from about 60 nm to about 190 nm, from about 60 nm to about 180 nm, from about 60 nm to about 170 nm, from about 60 nm to about 160 nm, 60 nm to about 150 nm, 60 nm to about 140 nm, 60 nm to about 130 nm, 60 nm to about 120 nm, 60 nm to about 110 nm, 60 nm to about 100 nm, 60 nm to about 90 nm, 60 nm to about 80 nm, or 60 nm to about 70 nm, for example about 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 160 nm, 170 nm, 180 nm, 190 nm, or 200 nm. Suitably, the Z-average size may be determined by DLS as commonly known in the art.

In preferred embodiments, the lipid-based carriers of the pharmaceutical composition of the invention (e.g. component B) have a Z-average size ranging from about 50 nm to about 150 nm, preferably in a range from about 50 nm to about 120 nm, more preferably in a range from about 60 nm to about 115 nm. Suitably, the Z-average size may be determined by DLS as commonly known in the art.

In embodiments, the lipid-based carriers have a Z-average size of less than about 150 nm, preferably less than about 120 nm, more preferably less than about 100 nm, most preferably less than about 80 nm. Suitably, the Z-average size may be determined by DLS as commonly known in the art.

In embodiments, the lipid-based carriers of the composition comprise more than about 70%, 75%, 80%, 85%, 90%, 91%, 92%, 93%, 94%, 95%, 96%, 97%, 98%, 99% lipid-based carriers that have a particle size ranging from about 50 nm to about 150 nm, preferably ranging from about 60 nm to about 115 nm, more preferably ranging from about 60 nm to about 80 nm. The particle size may be determined by DLS as commonly known in the art (e.g. MADLS). Alternatively, nanoparticle tracking analysis (NTA) or MFI as commonly known in the art may be used, or electron microscopy may be used (determining the particle size of lipid-based carriers comprised in a certain sample volume of the composition).

In embodiments, the lipid-based carriers of the composition comprise less than about 10%, 9%, 8%, 7%, 6%, 5%, 4%, 3%, 2%, 1% lipid-based carriers that have a particle size exceeding about 500 nm. The particle size may be determined by DLS as commonly known in the art (e.g. MADLS). Alternatively, nanoparticle tracking analysis (NTA) or MFI as commonly known in the art may be used, or electron microscopy may be used (determining the particle size of lipid-based carriers comprised in a certain sample volume of the composition).

In embodiments, the pharmaceutical composition comprising RNA formulated/encapsulated in lipid-based carriers comprises less than about 200,000 sub visible particles ≥2 μm (#per ml). In embodiments, the pharmaceutical composition comprising the lipid-based carriers encapsulating the RNA comprises less than about 100,000 sub visible particles ≥2 μm (#per ml). Preferably, the number of sub visible particles is determined by MFI.

In embodiments, the lipid-based carriers are a liposomes, lipid nanoparticles, lipoplexes, and/or nanoliposomes. Accordingly, the pharmaceutical composition of the pharmaceutical composition of the invention (e.g. component B) comprises liposomes, lipid nanoparticles, lipoplexes, and/or nanoliposomes that suitably encapsulate the RNA.

In preferred embodiments, the lipid-based carriers are lipid nanoparticles (LNPs). Accordingly, the pharmaceutical composition of the pharmaceutical composition of the pharmaceutical composition of the invention (e.g. component B) comprises lipid nanoparticles that suitably encapsulate the RNA.

In preferred embodiments, the RNA is completely or partially encapsulated in a lipid nanoparticle, wherein the RNA may be located in the interior space of the lipid nanoparticle, within the lipid layer/membrane of the lipid nanoparticle, or associated with the exterior surface of the lipid nanoparticle. Lipid nanoparticles (LNPs) are typically characterized as microscopic lipid particles having a solid (lipid) core or partially solid (lipid) core. Typically, an LNP does not comprise an interior aqua space sequestered from an outer medium by a bilayer. However, an LNP may comprise multiple internal (aqueous) droplets in the core of a lipid nanoparticle that may entrap the RNA. In an LNP, the RNA may suitably be encapsulated or incorporated in the lipid portion of the LNP enveloped by some or the entire lipid portion of the LNP. An LNP may comprise any lipid combination capable of forming a particle to which the RNA may be attached, or in which the RNA may be encapsulated.

In preferred embodiments, the lipid-based carriers (e.g. LNPs) of the pharmaceutical composition of the invention (e.g. component B) comprise at least two lipid components, at least three lipid components, preferably at least four lipid components, wherein the lipid components may be selected from at least one aggregation-reducing lipid, at least one cationic lipid, at least one neutral lipid, and/or at least one steroid or steroid analogue.

In preferred embodiments, the lipid-based carriers (e.g. LNPs) of the pharmaceutical composition of the invention (e.g. component B) comprise at least one aggregation-reducing lipid, at least one cationic lipid, at least one neutral lipid, and/or at least one steroid or steroid analogue.

In embodiments, the lipid-based carriers of the pharmaceutical composition of the invention (e.g. component B) comprise at least one aggregation reducing lipid.

In embodiments, the lipid-based carriers comprise the aggregation reducing lipid (e.g. polymer-conjugated lipid) in a molar ratio of about 0.5% to about 15%, preferably in a molar ratio of about 1.0% to about 2.5%, for example in a molar ratio of about 1.4%, about 1.5%, about 1.6%, about 1.7%, about 1.8%, about 1.9%. In preferred embodiments, the lipid-based carriers comprise the aggregation reducing lipid in a molar ratio of about 1.7% (based on 100% total moles of lipids in the lipid-based carriers).

In embodiments, the lipid-based carriers comprise the aggregation reducing lipid in a weight ratio of about 2% to about 10%, preferably in a weight ratio of about 4% to about 10%, for example in a weight ratio of about 5%, about 6%, about 7%, about 8%, about 9%. In embodiments, the lipid-based carriers comprise the aggregation reducing lipid in a weight ratio of about 6.97% (based on 100% total weight of lipids in the lipid-based carriers).

The term “aggregation reducing lipid” refers to a molecule comprising both a lipid portion and a moiety suitable of reducing or preventing aggregation of the lipid-based carriers in a composition. Under storage conditions, the lipid-based carriers may undergo charge-induced aggregation, a condition which can be undesirable for the stability of the composition. Therefore, it can be desirable to include a lipid compound which can reduce aggregation, for example by sterically stabilizing the lipid-based carriers. Such a steric stabilization may occur when a compound having a sterically bulky but uncharged moiety that shields or screens the charged portions of a lipid-based carriers from close approach to other lipid-based carriers in the composition. In the context of the invention, stabilization of the lipid-based carriers is achieved by including lipids which may comprise a lipid bearing a sterically bulky group which, after formation of the lipid-based carrier, is preferably located on the exterior of the lipid-based carrier. Suitable aggregation reducing groups include hydrophilic groups, e.g. polymers, such as poly(oxyalkylenes), e.g., a poly(ethylene glycol) or poly(propylene glycol). Lipids comprising a polymer as aggregation reducing group are herein referred to as “polymer conjugated lipid”.

In preferred embodiments, the aggregation reducing lipid of the lipid-based carriers of the pharmaceutical composition of the invention (e.g. component B) is a polymer conjugated lipid.

The term “polymer conjugated lipid” refers to a molecule comprising both a lipid portion and a polymer portion, wherein the polymer is suitable of reducing or preventing aggregation of lipid-based carriers comprising the RNA. A polymer has to be understood as a substance or material consisting of very large molecules, or macromolecules, composed of many repeating subunits. A suitable polymer in the context of the invention may be a hydrophilic polymer. An example of a polymer conjugated lipid is a PEGylated or PEG-conjugated lipid.

In preferred embodiments, the aggregation reducing lipid of the lipid-based carrier is selected from a polymer conjugated lipid. In preferred embodiments, the polymer conjugated lipid is a PEG-conjugated lipid (or PEGylated lipid or PEG lipid).

In certain embodiments, the lipid-based carriers comprise a polyethylene glycol-lipid (PEG-conjugated). Suitable polyethylene glycol-lipids include PEG-conjugated phosphatidylethanolamine, PEG-conjugated phosphatidic acid, PEG-conjugated ceramides (e.g. PEG-CerC14 or PEG-CerC20), PEG-conjugated dialkylamines, PEG-conjugated diacylglycerols, PEG-conjugated dialkylglycerols. Representative polyethylene glycol-lipids include PEG-c-DOMG, PEG-c-DMA, and PEG-s-DMG. In one embodiment, the polyethylene glycol-lipid is N-[(methoxy poly(ethylene glycol)2000)carbamyl]-1,2-dimyristyloxlpropyl-3-amine (PEG-c-DMA). In a preferred embodiment, the polyethylene glycol-lipid is PEG-2000-DMG. In one embodiment, the polyethylene glycol-lipid is PEG-c-DOMG). In other embodiments, the lipid-based carriers comprise a PEG-conjugated diacylglycerol (PEG-DAG) such as 1-(monomethoxy-polyethyleneglycol)-2,3-dimyristoylglycerol (PEG-DMG), a PEG-conjugated phosphatidylethanoloamine (PEG-PE), a PEG-conjugated succinate diacylglycerol (PEG-S-DAG) such as 4-O-(2′,3′-di(tetradecanoyloxy)propyl-1-O-(ω-methoxy(polyethoxy)ethyl)butanedioate (PEG-S-DMG), a PEG-conjugated ceramide (PEG-cer), or a PEG-conjugated dialkoxypropylcarbamate such as ω-methoxy(polyethoxy)ethyl-N-(2,3di(tetradecanoxy)propyl)carbamate or 2,3-di(tetradecanoxy)propyl-N-(ω-methoxy(polyethoxy)ethyl)carbamate, or 1,2-dimyristoyl-rac-glycero-3-methoxypolyethylene glycol-2000 (PEG2000 DMG).

In preferred embodiments, the polymer conjugated lipid, e.g. the PEG-conjugated lipid is 1,2-dimyristoyl-rac-glycero-3-methoxypolyethylene glycol-2000 (PEG2000 DMG).

In other preferred embodiments, the polymer conjugated lipid, e.g. the PEG-conjugated lipid is preferably derived from formula (IV) of published PCT patent application WO2018/078053A1. Accordingly, the PEG-conjugated lipids derived from formula (IV) of published PCT patent application WO2018/078053A1, and the respective disclosure relating thereto, are herewith incorporated by reference.

In preferred embodiments, the lipid-based carriers (e.g. the LNPs) encapsulating the RNA comprise a polymer conjugated lipid, preferably a PEG-conjugated, wherein the a PEG-conjugated lipid is preferably derived from formula (IVa) of published PCT patent application WO2018/078053A1. Accordingly, a PEG-conjugated lipids derived from formula (IVa) of published PCT patent application WO2018/078053A1, and the respective disclosure relating thereto, are herewith incorporated by reference.

In particularly preferred embodiments, the lipid-based carriers (e.g. the LNPs) of the pharmaceutical composition of the invention (e.g. component B) comprise a PEG-conjugated lipid, wherein said PEG-conjugated lipid is a lipid according to formula (IVa) or derived from formula (IVa):

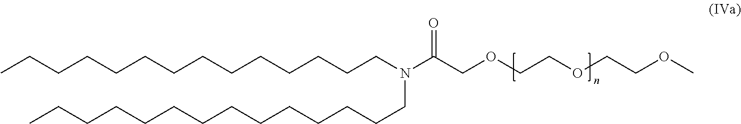

wherein n has a mean value ranging from 30 to 60, such as about 30±2, 32±2, 34±2, 36±2, 38±2, 40±2, 42±2, 44±2, 46±2, 48±2, 50±2, 52±2, 54±2, 56±2, 58±2, or 60±2. In a preferred embodiment n is about 49. In another preferred embodiment n is about 45. Suitably, the aggregation reducing lipid according to formula (IVa) is ALC-0159.

Further examples of PEG-conjugated lipids suitable in that context are provided in US2015/0376115A1 and WO2015/199952, each of which is incorporated by reference in its entirety.

Furthermore, in a specific embodiment, the lipid based carrier encapsulating the RNA comprise a polymer conjugated lipid, preferably a PEG-conjugated lipid, wherein said PEG-conjugated lipid is 1,2-dimyristoyl-rac-glycero-3-methoxypolyethylene glycol 2000 (DMG-PEG 2000) according to or derived from the following structure:

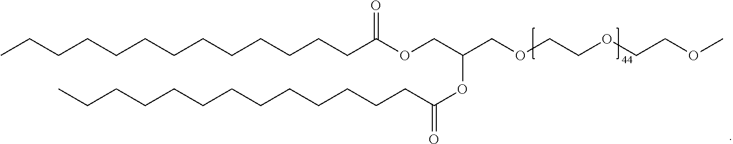

As used in the art, “DMG-PEG 2000” is considered a mixture of 1,2-DMG PEG2000 and 1,3-DMG PEG2000 in ˜97:3 ratio.

According to preferred embodiments of the invention, the aggregation reducing lipid of the lipid-based carrier is a PEG-conjugated lipid selected or derived from DMG-PEG 2000, C10-PEG2K, Cer8-PEG2K, or ALC-0159 (lipid of formula IVa), preferably ALC-0159.

In preferred embodiments, the lipid-based carriers of the pharmaceutical composition of the invention (e.g. component B) comprise at least one cationic lipid.

In various embodiments, the lipid-based carriers of the pharmaceutical composition of the invention (e.g. component B) comprise the cationic lipid in a molar ratio of about 20% to about 60%, preferably in a molar ratio of about 38% to about 57%, for example in a molar ratio of about 42%, 43%, 44%, 45%, 46%, 47%, 48%, 49%, 50%, 51%, or about 52% (based on 100% total moles of lipids in the lipid-based carriers). In preferred embodiments, the lipid-based carriers comprise a cationic lipid in a molar ratio of about 47.4% (based on 100% total moles of lipids in the lipid-based carriers).

In embodiments, the lipid-based carriers of the pharmaceutical composition of the invention (e.g. component B) comprise the cationic lipid in a weight ratio of about 24% to about 72%, preferably in a weight ratio of about 45% to about 68%, for example in a weight ratio of about 50%, 51%, 52%, 53%, 54%, 55%, 56%, 57%, 58%, 59%, 60%, 61%, or about 62% (based on 100% total weight of lipids in the lipid-based carriers). In preferred embodiments, the lipid-based carriers comprise the cationic lipid in a weight ratio of about 56.28% (based on 100% total weight of lipids in the lipid-based carriers).

The cationic lipid of the lipid-based carriers may be cationisable, i.e. it becomes protonated as the pH is lowered below the pK of the ionizable group of the lipid, but is progressively more neutral at higher pH values. At pH values below the pK, the lipid is then able to associate with negatively charged nucleic acids. In certain embodiments, the cationic lipid comprises a zwitterionic lipid that assumes a positive charge on pH decrease.

Suitable cationic lipids or cationisable lipids include, but are not limited to, DSDMA, N,N-dioleyl-N,N-dimethylammonium chloride (DODAC), N,N-distearyl-N,N-dimethylammonium bromide (DDAB), 1,2-dioleoyltrimethyl ammonium propane chloride (DOTAP) (also known as N-(2,3-dioleoyloxy)propyl)-N,N,N-trimethylammonium chloride and 1,2-Dioleyloxy-3-trimethylaminopropane chloride salt), N-(1-(2,3-dioleyloxy)propyl)-N,N,N-trimethylammonium chloride (DOTMA), N,N-dimethyl-2,3-dioleyloxy)propylamine (DODMA), ckk-E12, ckk, 1,2-DiLinoleyloxy-N,N-dimethylaminopropane (DLinDMA), 1,2-Dilinolenyloxy-N,N-dimethylaminopropane (DLenDMA), 1,2-di-y-linolenyloxy-N,N-dimethylaminopropane (γ-DLenDMA), 98N12-5, 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.CI), ICE (Imidazol-based), HGT5000, HGT5001, DMDMA, CLinDMA, CpLinDMA, DMOBA, DOcarbDAP, DLincarbDAP, DLinCDAP, KLin-K-DMA, DLin-K-XTC2-DMA, XTC (2,2-Dilinoleyl-4-dimethylaminoethyl-[1,3]-dioxolane) HGT4003, 1,2-Dilinoleoyl-3-trimethylaminopropane chloride salt (DLin-TAP.CI), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DM A), 2,2-Dilinoleyl-4-dimethylaminomethyl-[1,3]-dioxolane (DLin-K-DMA) or analogs thereof, (3aR,5s,6aS)—N,N-dimethyl-2,2-di((9Z,12Z)-octadeca-9,12-dienyl)tetrahydro-3aH-cyclopenta[d][1,3]dioxol-5-amine, (6Z,9Z,28Z,31 Z)-heptatriaconta-6,9,28,31-tetraen-19-yl-4-(dimethylamino)butanoate (MC3), ALNY-100 ((3aR,5s,6aS)—N,N-dimethyl-2,2-di((9Z,12Z)-octadeca-9,12-dienyl)tetrahydro-3aH-cyclopenta[d][1,3]dioxol-5-amine)), 1,1′-(2-(4-(2-((2-(bis(2-hydroxydodecyl)amino)ethyl)(2-hydroxydodecyl)amino)ethyl)piperazin-1-yl)ethylazanediyl)didodecan-2-ol (C12-200), 2,2-dilinoleyl-4-(2-dimethylaminoethyl)-[1,3]-dioxolane (DLin-K-C2-DMA), 2,2-dilinoleyl-4-dimethylaminomethyl-[1,3]-dioxolane (DLin-K-DMA), NC98-5 (4,7, 13-tris(3-oxo-3-(undecylamino)propyl)-N,N 16-diundecyl-4,7, 10,13-tetraazahexadecane-1,16-diamide), (6Z,9Z,28Z,31Z)-heptatriaconta-6,9,28,31-tetraen-19-yl 4-(dimethylamino) butanoate (DLin-M-C3-DMA), 3-((6Z,9Z,28Z,31Z)-heptatriaconta-6,9,28,31-tetraen-19-yloxy)-N,N-dimethylpropan-1-amine (MC3 Ether), 4-((6Z,9Z,28Z,31 Z)-heptatriaconta-6,9,28,31-tetraen-19-yloxy)-N,N-dimethylbutan-1-amine (MC4 Ether), LIPOFECTIN® (commercially available cationic liposomes comprising DOTMA and 1,2-dioleoyl-sn-3phosphoethanolamine (DOPE), from GIBCO/BRL, Grand Island, N.Y.); LIPOFECTAMINE® (commercially available cationic liposomes comprising N-(1-(2,3dioleyloxy)propyl)-N-(2-(sperminecarboxamido)ethyl)-N,N-dimethylammonium trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.) or any combination of any of the foregoing. Further suitable cationic or cationizable lipids include those described in international patent publications WO2010/053572 (and particularly, CI 2-200 described at paragraph [00225]) and WO2012/170930, both of which are incorporated herein by reference, HGT4003, HGT5000, HGTS001, HGT5001, HGT5002 (see US20150140070A1).

In embodiments, the cationic lipid of the lipid-based carriers is selected from at least one amino lipid.

Suitable amino lipids include, but are not limited to, 1,2-dilinoleyoxy-3-(dimethylamino)acetoxypropane (DLin-DAC), 1,2-dilinoleyoxy-3morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3dimethylaminopropane (DLin-2-DMAP), 1,2-dilinoleyloxy-3-trimethylaminopropane chloride salt (DLin-TMA.CI), 1,2-dilinoleoyl-3-trimethylaminopropane chloride salt (DLin-TAP.CI), 1,2-dilinoleyloxy-3-(N-methylpiperazino)propane (DLin-MPZ), 3-(N,Ndilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), and 2,2-dilinoleyl-4-dimethylaminomethyl-[1,3]-dioxolane (DLin-K-DMA), 2,2-dilinoleyl-4-(2-dimethylaminoethyl)-[1,3]-dioxolane (DLin-KC2-DMA); dilinoleyl-methyl-4-dimethylaminobutyrate (DLin-MC3-DMA); MC3 (US20100324120).

In embodiments, the cationic or cationizable lipid of the lipid-based carriers is selected from at least one aminoalcohol lipidoid.

Aminoalcohol lipidoids which may be used in the present invention may be prepared by the methods described in U.S. Pat. No. 8,450,298, herein incorporated by reference in its entirety. Suitable (ionizable) lipids can also be the compounds as disclosed in Tables 1, 2 and 3 and as defined in claims 1-24 of WO2017/075531 A1, hereby incorporated by reference.

In other embodiments, suitable cationic or cationizable lipids may be selected from compounds as disclosed in WO2015/074085A1 (i.e. ATX-001 to ATX-032 or the compounds as specified in claims 1-26), U.S. Appl. Nos. 61/905,724 and Ser. No. 15/614,499 or U.S. Pat. Nos. 9,593,077 and 9,567,296 hereby incorporated by reference in their entirety. In other embodiments, suitable cationic or cationizable lipids may be selected from compounds as disclosed in WO2017/117530A1 (i.e. lipids 13, 14, 15, 16, 17, 18, 19, 20, or the compounds as specified in the claims), hereby incorporated by reference in its entirety.

In preferred embodiments, cationic or cationizable lipids may be selected from the lipids disclosed in WO2018/078053A1 (i.e. lipids derived from formula I, II, and III of WO2018/078053A1, or lipids as specified in Claims 1 to 12 of WO2018/078053A1), the disclosure of WO2018/078053A1 hereby incorporated by reference in its entirety. In that context, lipids disclosed in Table 7 of WO2018/078053A1 (e.g. lipids derived from formula I-1 to I-41) and lipids disclosed in Table 8 of WO2018/078053A1 (e.g. lipids derived from formula II-1 to II-36) may be suitably used. Accordingly, formula I-1 to formula I-41 and formula II-1 to formula II-36 of WO2018/078053A1, and the specific disclosure relating thereto, are herewith incorporated by reference.

In preferred embodiments, suitable cationic or cationizable lipids may be derived from formula III of published PCT patent application WO2018/078053A1. Accordingly, formula III of WO2018/078053A1, and the specific disclosure relating thereto, are herewith incorporated by reference.

Accordingly, the lipid-based carriers (e.g. LNPs) of the pharmaceutical composition of the invention (e.g. component B) comprise a cationic lipid according to formula (III) or derived from formula (III):

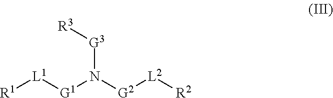

Formula (III) is further defined in that:
one of L1 or L2 is —O(C═O)—, —(C═O)O—, —C(═O)—, —O—, —S(O)x-, —S—S—, —C(═O)S—, SC(═O)—, —NRaC(═O)—, —C(═O)NRa—, —NRaC(═O)NRa—, —OC(═O)NRa— or —NRaC(═O)O—, and the other of L1 or L2 is —O(C═O)—, —(C═O)O—, —C(═O)—, —O—, —S(O)x-, —S S—, —C(═O)S—, SC(═O)—, —NRaC(═O)—, —C(═O)NRa—, —NRaC(═O)NRa—, —OC(═O)NRa— or —NRaC(═O)O— or a direct bond;
G1 and G2 are each independently unsubstituted C1-C12 alkylene or C1-C12 alkenylene;
G3 is C1-C24 alkylene, C1-C24 alkenylene, C3-C8 cycloalkylene, C3-C8 cycloalkenylene;
Ra is H or C1-C12 alkyl;
R1 and R2 are each independently C6-C24 alkyl or C6-C24 alkenyl;
R3 is H, OR5, CN, C(═O)OR4, OC(═O)R4 or —NR5C(═O)R4;
R4 is C1-C12 alkyl;
R5 is H or C1-C6 alkyl; and
x is 0, 1 or 2.

In particularly preferred embodiments, the lipid-based carriers (e.g. LNPs) of the pharmaceutical composition of the invention (e.g. component B) comprise a cationic lipid selected from or derived from structures III-1 to III-36 of Table 9 of published PCT patent application WO2018/078053A1. Accordingly, formula III-1 to III-36 of WO2018/078053A1, and the specific disclosure relating thereto, are herewith incorporated by reference.

In particularly preferred embodiments, the lipid-based carriers (e.g. LNPs) of the pharmaceutical composition of the invention (e.g. component B) comprise a cationic lipid according to formula (III-3) or derived from formula (III-3):

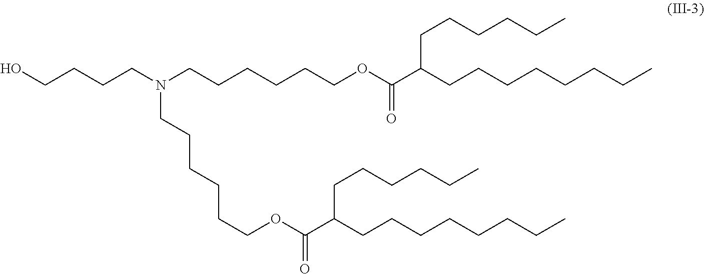 [SMILES: CCCCCCCCC(CCCCCC)C(=O)OCCCCCCN(CCCCO)CCCCCCOC(=O)C(CCCCCC)CCCCCCCC]

A preferred lipid of formula III-3 in the context of the invention is ALC-0315.

In certain embodiments, the cationic lipid as defined herein, more preferably cationic lipid compound III-3, is present in the lipid-based carriers of the pharmaceutical composition of the invention (e.g. component B) in an amount from about 30 to about 95 mole percent, relative to the total lipid content of the lipid-based carrier. If more than one cationic lipid is incorporated within the lipid-based carrier, such percentages apply to the combined cationic lipids.

In some embodiments, the lipid-based carriers comprise a cationic lipid resembled by the cationic lipid COATSOME® SS-EC (former name: SS-33/4PE-15; NOF Corporation, Tokyo, Japan), in accordance with the following formula

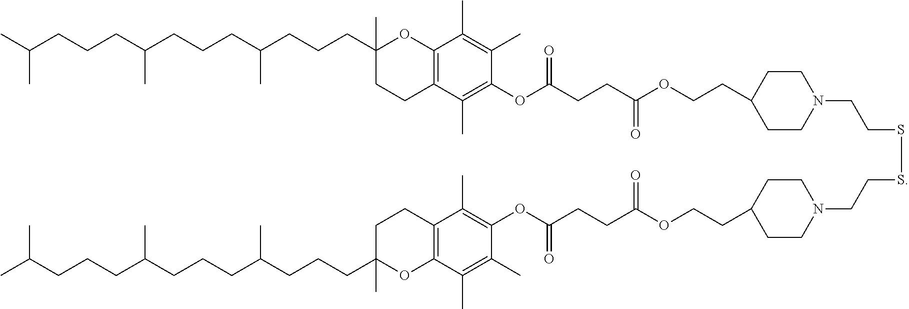 [SMILES: Cc1c(C)c2c(c(C)c1OC(=O)CCC(=O)OCCC1CCN(CCSSCCN3CCC(CCOC(=O)CCC(=O)Oc4c(C)c(C)c5c(c4C)CCC(C)(CCCC(C)CCCC(C)CCCC(C)C)O5)CC3)CC1)CCC(C)(CCCC(C)CCCC(C)CCCC(C)C)O2]

As described further below, those lipid nanoparticles are termed “GN01”.

In some embodiments, the lipid-based carriers comprise a cationic lipid according to or derivable from the following formula (Heptadecan-9-yl 8-((2-hydroxyethyl)(6-oxo-6-(undecyloxy)hexyl)amino)octanoate; SM-102):

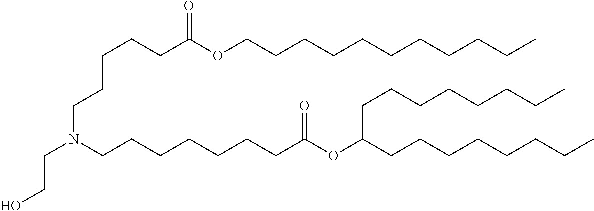 [SMILES: CCCCCCCCCCCOC(=O)CCCCCN(CCO)CCCCCCCC(=O)OC(CCCCCCCC)CCCCCCCC]

Other suitable (cationic or ionizable) lipids that may be comprised in the lipid-based carriers are disclosed in WO2009/086558, WO2009/127060, WO2010/048536, WO2010/054406, WO2010/088537, WO2010/129709, WO2011/153493, WO 2013/063468, US2011/0256175, US2012/0128760, US2012/0027803, U.S. Pat. No. 8,158,601, WO2016/118724, WO2016/118725, WO2017/070613, WO2017/070620, WO2017/099823, WO2012/040184, WO2011/153120, WO2011/149733, WO2011/090965, WO2011/043913, WO2011/022460, WO2012/061259, WO2012/054365, WO2012/044638, WO2010/080724, WO2010/21865, WO2008/103276, WO2013/086373, WO2013/086354, U.S. Pat. Nos. 7,893,302, 7,404,969, 8,283,333, 8,466,122 and 8,569,256 and US Patent Publication No. US2010/0036115, US2012/0202871, US2013/0064894, US2013/0129785, US2013/0150625, US2013/0178541, US2013/0225836, US2014/0039032 and WO2017/112865. In that context, the disclosures of WO2009/086558, WO2009/127060, WO2010/048536, WO2010/054406, WO2010/088537, WO2010/129709, WO2011/153493, WO 2013/063468, US2011/0256175, US2012/0128760, US2012/0027803, U.S. Pat. No. 8,158,601, WO2016/118724, WO2016/118725, WO2017/070613, WO2017/070620, WO2017/099823, WO2012/040184, WO2011/153120, WO2011/149733, WO2011/090965, WO2011/043913, WO2011/022460, WO2012/061259, WO2012/054365, WO2012/044638, WO2010/080724, WO2010/21865, WO2008/103276, WO2013/086373, WO2013/086354, U.S. Pat. Nos. 7,893,302, 7,404,969, 8,283,333, 8,466,122 and 8,569,256 and US Patent Publication No. US2010/0036115, US2012/0202871, US2013/0064894, US2013/0129785, US2013/0150625, US2013/0178541, US2013/0225836 and US2014/0039032 and WO2017/112865 specifically relating to (cationic) lipids suitable for LNPs (or liposomes, nanoliposomes, lipoplexes) are incorporated herewith by reference.

In preferred embodiments of the invention, the at least one cationic lipid of the lipid-based carrier is lipid selected or derived from ALC-0315 (lipid of formula III), SM-102, SS-33/4PE-15, HEXA-CSDE-PipSS, or compound C26, preferably ALC-0315. In embodiments, amino or cationic lipids as defined herein have at least one protonatable or deprotonatable group, such that the lipid is positively charged at a pH at or below physiological pH (e.g. pH 7.4), and neutral at a second pH, preferably at or above physiological pH. It will, of course, be understood that the addition or removal of protons as a function of pH is an equilibrium process, and that the reference to a charged or a neutral lipid refers to the nature of the predominant species and does not require that all of lipids have to be present in the charged or neutral form. Lipids having more than one protonatable or deprotonatable group, or which are zwitterionic, are not excluded and may likewise suitable in the context of the present invention. In some embodiments, the protonatable lipids have a pKa of the protonatable group in the range of about 4 to about 11, e.g., a pKa of about 5 to about 7.

In specific embodiments, the lipid-based carriers of the invention comprise two or more (different) cationic lipids as defined herein.

In preferred embodiments, the lipid-based carriers of the pharmaceutical composition of the invention (e.g. component B) comprise at least one neutral lipid.

In preferred embodiments, the lipid-based carriers of the pharmaceutical composition of the invention (e.g. component B) comprise a neutral lipid in a molar ratio of about 5% to about 25%, preferably in a molar ratio of about 8% to about 12%, for example in a molar ratio of about 9%, 9.5%, 10%, 10.5% or 11%. In preferred embodiments, the lipid-based carriers comprise a neutral lipid in a molar ratio of about 10% (based on 100% total moles of lipids in the lipid-based carriers).

In other preferred embodiments, the lipid-based carriers of the pharmaceutical composition of the invention (e.g. component B) comprise the neutral lipid in a weight ratio of about 3% to about 20%, preferably in a weight ratio of about 9% to about 15%, for example in a weight ratio of about 10%, about 11%, about 12%, about 13%, about 14%. In embodiments, the lipid-based carriers comprise the neutral lipid in a weight ratio of about 12.24% (based on 100% total weight of lipids in the lipid-based carriers).

In various embodiments, the molar ratio of the cationic lipid to the neutral lipid ranges from about 2:1 to about 8:1.

The term “neutral lipid” refers to any one of a number of lipid species that exist in either an uncharged or neutral zwitterionic form at physiological pH. Suitable neutral lipids include diacylphosphatidylcholines, diacylphosphatidylethanolamines, ceramides, sphingomyelins, dihydro sphingomyelins, cephalins, and cerebrosides.

In embodiments, the lipid-based carriers of the pharmaceutical composition of the invention (e.g. component B) comprises one or more neutral lipids, wherein the neutral lipid is selected from the group comprising distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearioyl-2-oleoylphosphatidyethanol amine (SOPE), and 1,2-dielaidoyl-sn-glycero-3-phophoethanolamine (transDOPE), or mixtures thereof. In some embodiments, the lipid-based carriers encapsulating the RNA comprise a neutral lipid selected from DSPC, DPPC, DMPC, DOPC, POPC, DOPE and SM.

In preferred embodiments, the neutral lipid of the lipid-based carriers of the pharmaceutical composition of the invention (e.g. component B) is 1,2-distearoyl-sn-glycero-3-phosphocholine (DSPC).

In other embodiments, the lipid-based carriers of the pharmaceutical composition comprise a neutral lipid being resembled by the structure 1,2-diphytanoyl-sn-glycero-3-phosphoethanolamine (DPhyPE):

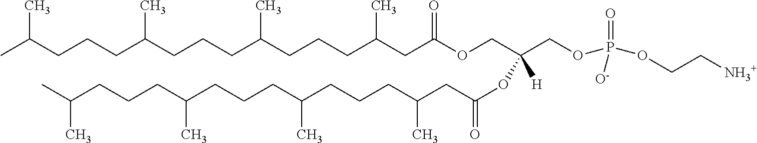 [SMILES: CC(C)CCCC(C)CCCC(C)CCCC(C)CC(=O)OC[C@H](COP(=O)([O-])OCC[NH3+])OC(=O)CC(C)CCCC(C)CCCC(C)CCCC(C)C]

In preferred embodiments of the invention, the at least one neutral lipid of the lipid-based carrier is lipid selected or derived from DSPC, DHPC, or DphyPE, preferably DSPC.

In preferred embodiments, the lipid-based carriers of the pharmaceutical composition comprise at least one steroid or steroid analogue.

In embodiments, the lipid-based carriers of the pharmaceutical composition comprises a steroid or steroid analogue in a molar ratio of about 25% to about 55%, preferably in a molar ratio of about 33% to about 49%, for example in a molar ratio of about 38%, 39%, 40%, 41%, 42%, 43%, or about 44%. In preferred embodiments, the lipid-based carriers comprise a steroid or steroid analogue in a molar ratio of about 40.9% (based on 100% total moles of lipids in the carriers).

In embodiments, the lipid-based carriers of the pharmaceutical composition comprise the steroid or steroid analogue in a weight ratio of about 6% to about 40%, preferably in a weight ratio of about 18% to about 30%, for example in a weight ratio of about 21%, about 22%, about 23%, about 24%, about 25%, about 26%, or about 27%. In preferred embodiments, the lipid-based carriers comprise a steroid or steroid analogue in a weight ratio of about 24.51% (based on 100% total weight of lipids in the lipid-based carriers).

Suitably, the molar ratio of the cationic lipid to steroid or steroid analogue may be in the range from about 2:1 to about 1:1.

In preferred embodiments, the steroid or steroid analogue of the lipid-based carriers of the pharmaceutical composition of the invention (e.g. component B) is cholesterol or cholesteryl hemisuccinate, preferably cholesterol.

In some embodiments, the cholesterol is a polymer conjugated cholesterol or a PEGylated cholesterol.

In preferred embodiments, the lipid-based carriers of the pharmaceutical composition, preferably the LNPs, comprise: (a) the RNA as defined herein, (b) a cationic lipid as defined herein, (c) the aggregation reducing lipid (such as a PEG-conjugated lipid) as defined herein, (d) optionally, a non-cationic lipid (such as a neutral lipid) as defined herein, and (e) optionally, a steroid or steroid analogue as defined herein.

In preferred embodiments, the cationic lipids (as defined above), non-cationic lipids (as defined above), cholesterol (as defined above), and/or aggregation reducing lipid (as defined above) may be combined at various relative molar ratios. For example, the ratio of cationic lipid to non-cationic lipid to cholesterol-based lipid to aggregation reducing lipid (e.g. PEG-conjugated lipid) may be between about 30-60:20-35:20-30:1-15, or at a ratio of about 40:30:25:5, 50:25:20:5, 50:27:20:3, 40:30:20:10, 40:32:20:8, 40:32:25:3 or 40:33:25:2, or at a ratio of about 50:25:20:5, 50:20:25:5, 50:27:20:3 40:30:20:10, 40:30:25:5 or 40:32:20:8, 40:32:25:3 or 40:33:25:2, respectively.

In preferred embodiments, the lipid-based carriers of the pharmaceutical composition comprise a lipid of formula (III) of WO2018/078053A1, at least one RNA as defined herein, a neutral lipid, a steroid, and a Polymer-conjugated lipid. In preferred embodiments, the lipid of formula (III) is lipid compound III-3, the neutral lipid is DSPC, the steroid is cholesterol, and the PEG-conjugated lipid is the compound of formula (IVa).

In particularly preferred embodiments, the lipid-based carriers comprising the RNA comprise
(i) at least one cationic lipid as defined herein, preferably a lipid of formula (III) of WO2018/078053A1, more preferably a lipid according to formula (III-3) or derived from formula (III-3), in particular ALC-0315;
(ii) at least one neutral lipid as defined herein, preferably 1,2-distearoyl-sn-glycero-3-phosphocholine (DSPC);
(iii) at least one steroid or steroid analog as defined herein, preferably cholesterol; and
(iv) at least one aggregation reducing lipid, preferably a polymer-conjugated lipid, more preferably a PEG-conjugated lipid according to formula (IVa) or derived from formula (IVa), in particular ALC-0159;
preferably wherein the lipid-based carriers encapsulate the RNA.

In particularly preferred embodiments, the lipid-based carriers comprising the RNA comprise (i) the cationic lipid ALC-0315 (lipid of formula III), (ii) the neutral lipid DSPC, (iii) cholesterol, and (iv) the aggregation reducing lipid ALC-0159 (lipid of formula IVa).

In particularly preferred embodiments, the lipid-based carriers comprising the RNA comprise (i) the cationic lipid SM-102, (ii) the neutral lipid DSPC, (iii) cholesterol, and (iv) the aggregation reducing lipid DMG-PEG 2000.

In preferred embodiments, the lipid-based carriers comprise (i) to (iv) in a molar ratio of about 20-60% cationic lipid, about 5-25% neutral lipid, about 25-55% steroid or steroid analogue, and about 0.5-15% aggregation reducing lipid, e.g. polymer conjugated lipid, preferably wherein the lipid-based carriers encapsulate the RNA.

In specific embodiments, the lipid-based carriers comprise or consist (i) to (iv) in a molar ratio of about 47.4% cationic lipid, about 10% neutral lipid, about 40.9% steroid or steroid analogue, and about 1.7% aggregation reducing lipid, e.g. polymer conjugated lipid, preferably wherein the lipid-based carriers encapsulate the RNA.

In preferred embodiments, the lipid-based carriers comprise (i) to (iv) in a weight ratio of about 30-70% cationic lipid, about 5-25% neutral lipid, about 10-40% steroid or steroid analogue, and about 2-20% aggregation reducing lipid, e.g. polymer conjugated lipid, preferably wherein the lipid-based carriers encapsulate the RNA.

In specific embodiments, the lipid-based carriers comprise or consist (i) to (iv) in a weight ratio of about 56.28% cationic lipid, about 12.24% neutral lipid, about 24.51% steroid or steroid analogue, and about 6.97% aggregation reducing lipid, preferably wherein the lipid-based carriers encapsulate the RNA.

In embodiments, the composition comprises the lipid-based carriers encapsulating the RNA which have a molar ratio of approximately 50:10:38.5:1.5, preferably 47.5:10:40.8:1.7 or more preferably 47.4:10:40.9:1.7 (i.e. proportion (mol %) of cationic lipid (preferably lipid III-3), DSPC, cholesterol, and aggregation reducing lipid (e.g. polymer conjugated lipid, preferably PEG-lipid (preferably PEG-lipid of formula (IVa) with n=49 or with n=45))).

In embodiments, the composition comprises lipid-based carriers encapsulating the RNA which have a weight ratio of approximately 56:12:25:7, preferably 56.3:12.2:24.5:7 or more preferably 56.28:12.24:24.51:6.97 (i.e. proportion (weight %) of cationic lipid (preferably lipid III-3), DSPC, cholesterol and aggregation reducing lipid (e.g. polymer conjugated lipid, preferably PEG-lipid (preferably PEG-lipid of formula (IVa) with n=49 or with n=45))).

In a specific embodiment, the lipid-based carriers encapsulating the RNA is a GN01 lipid nanoparticle comprising a cationic lipid SS-EC, a neutral lipid 1,2-diphytanoyl-sn-glycero-3-phosphoethanolamine (DPhyPE), cholesterol, and a polymer conjugated lipid 1,2-dimyristoyl-rac-glycero-3-methoxypolyethylene glycol (PEG-DMG).

In embodiments, the GN01 lipid nanoparticles comprise:
(a) cationic lipid SS-EC (former name: SS-33/4PE-15; NOF Corporation, Tokyo, Japan) at an amount of 45-65 mol %;
(b) cholesterol at an amount of 25-45 mol %;
(c) DPhyPE at an amount of 8-12 mol %; and
(d) PEG-DMG 2000 at an amount of 1-3 mol %;
each amount being relative to the total molar amount of all lipidic excipients of the GN01 lipid nanoparticles.

In a preferred embodiment, the GN01 lipid nanoparticles comprises 59 mol % cationic lipid, 10 mol % neutral lipid, 29.3 mol % steroid and 1.7 mol % polymer conjugated lipid, preferably pegylated lipid. In a most preferred embodiment, the GN01 lipid nanoparticles comprise 59 mol % cationic lipid SS-EC, 10 mol % DPhyPE, 29.3 mol % cholesterol and 1.7 mol % DMG-PEG 2000.

In a preferred embodiment, the GN01 lipid nanoparticles comprise 59 mol % cationic lipid COATSOME® SS-EC (former name: SS-33/4PE-15 as apparent from the examples section; NOF Corporation, Tokyo, Japan), 29.3 mol % cholesterol as steroid, 10 mol % DPhyPE as neutral lipid/phospholipid and 1.7 mol % DMG-PEG 2000 as polymer conjugated lipid. For GN01 lipid nanoparticles, N/P (lipid to nucleic acid, e.g. RNA mol ratio) preferably is 14 and total lipid/RNA mass ratio preferably is 40 (m/m).

In other embodiments, the lipid-based carriers encapsulating the RNA comprise the cationic lipid DLin-KC2-DMA (50 mol %) or DLin-MC3-DMA (50 mol %), the neutral lipid DSPC (10 mol %), the aggregation reducing lipid PEG-DOMG (1.5 mol %) and the structural lipid is cholesterol (38.5 mol %).

In other embodiments, the lipid-based carriers encapsulating the RNA comprises the cationic/ionizable lipid Heptadecan-9-yl 8-((2-hydroxyethyl)(6-oxo-6-(undecyloxy)hexyl)amino)octanoate (SM-102), the neutral lipid 1,2-distearoyl-sn-glycero-3 phosphocholine (DSPC), the aggregation reducing lipid 1,2-dimyristoyl-rac-glycero-3-methoxypolyethylene glycol-2000 (PEG2000 DMG), and cholesterol, preferably at mol % 50/10/1.5/38.5.

In other embodiments, the lipid-based carrier may be selected from any lipid-based carrier as described in WO2019/222424, WO2019/226925, WO2019/232095, WO2019/232097, or WO2019/232208, the disclosure of WO2019/222424, WO2019/226925, WO2019/232095, WO2019/232097, or WO2019/232208 relating to lipid-based carriers herewith incorporated by reference.

In other embodiments, the lipid-based carrier encapsulating the RNA may be composed of three lipid components, preferably imidazole cholesterol ester (ICE), 1,2-dioleoyl-sn-glycero-3-phosphoethanolamine (DOPE), and the aggregation reducing lipid 1,2-dimyristoyl-sn-glycerol, methoxypolyethylene glycol (DMG-PEG-2K).

In embodiments, the lipid-based carriers of the pharmaceutical composition is a purified lipid-based carrier. Accordingly, the lipid-based carriers have been purified by at least one purification step. Such a purification step may suitably selected from at least one step of tangential flow filtration and/or at least one step of clarification and/or at least one step of filtration.

The term “purified lipid-based carrier” as used herein has to be understood as lipid-based carriers comprising the RNA, optionally encapsulating the RNA, which have a higher purity after certain purification steps (e.g. tangential flow filtration, clarification filtration, chromatography steps) as compared to the starting material. Typical impurities that are essentially not present in the purified lipid-based carriers comprise e.g. free lipids, organic solvents, empty lipid-based carriers (without RNA cargo), fused lipid-based carriers (lipid-based carriers exceeding the desired size), small micelles (lipid-based carriers that are smaller than the desired size), lipid-based carriers that do not comprise the desired components (e.g. lacking the aggregation reducing lipid), lipid degradation products etc. Other potential impurities may be derived from the synthesis of the individual lipid components. Accordingly, the lipid components used in formulating the lipid-based carriers have a purity level of at least 80%, preferably at least 90%, more preferably at least 95%. It is desirable for the “degree of lipid-based carrier purity” to be as close as possible to 100%. “Purified lipid-based carriers” as used herein have a degree of purity of more than 75%, 80%, 85%, very particularly 90%, 91%, 92%, 93%, 94%, 95%, 96%, 97%, 98% and most favourably 99% or more. The degree of purity may for example be determined by an analytical HPLC (to determine contaminations and to determine the lipid ratio in the carrier) or by determining the size and size distribution of the obtained lipid-based carriers (e.g. using DLS, NTA, MFI) or the shape of the lipid carriers (e.g. by EM analysis).

In various embodiments, the pharmaceutical composition comprising RNA formulated in lipid-based carriers has a certain clarity, e.g. without showing signs of increased turbidity, e.g. caused by contacting the composition with a syringe. The detection of a certain turbidity may be an indicator for agglomeration of lipid-based carriers (or RNA agglomeration) or precipitation of lipid-based carriers (or RNA precipitation). Accordingly the pharmaceutical composition having the desired quality according to the invention is typically clear. An reduced quality of the composition is visible, for example, as turbidity within the pharmaceutical composition, wherein increasing turbidity may be correlated with decreasing product quality and decreasing stability, which may eventually result in the formation of precipitates or agglomerates. An increase of turbidity may be caused by e.g. a storage of the pharmaceutical composition in a syringe that is not suitable and not compatible with said composition.

Turbidity is the measure of relative clarity of a liquid. It is an optical characteristic of a liquid composition that is a measurement of the amount of light that is scattered by material in the water when a light is shined through the water sample. The higher the intensity of scattered light, the higher the turbidity. Turbidity may be measured at 860 nm with a detecting angle of 90° using commercially available instruments and methods known in the art. An example for a commercially available instrument is a NEPHLA turbidimeter, available from Dr. Lange, Düsseldorf, Germany. The system is calibrated with formazin as standard and the results were given in formazin nephelometric units (FNU).

In embodiments, the pharmaceutical composition comprising RNA formulated in lipid-based carriers has a turbidity ranging from about 150 FNU to about 0.0 FNU. In embodiments, the composition has a turbidity of about 100 FNU or less, preferably of about 50 FNU or less, more preferably of about 25 FNU or less.

In preferred embodiments, the pharmaceutical composition comprising RNA, preferably formulated in lipid-based carriers, comprises less than about 500 ppM ethanol, preferably less than about 50 ppM ethanol, more preferably less than about 5 ppM ethanol. Ethanol may represent a contamination e.g. in embodiments where the RNA is formulated in lipid-based carriers as typically lipids are provided as ethanolic lipid compositions in respective formulation methods. It is preferred that the pharmaceutical composition comprising RNA comprises a low amount of ethanol (or other organic solvent) to reduce the risk of extracting compounds from a syringe (e.g. extractable lubricants).

In embodiments, the pharmaceutical composition comprises a buffer e.g. comprising a sugar and/or a salt and/or a buffering agent.

In preferred embodiments, the pharmaceutical composition further comprises a salt, preferably NaCl. In embodiments, the concentration of the salt comprised in the composition is in a range from about 10 mM to about 300 mM, preferably about 150 mM. In embodiments, the salt comprised in composition is NaCl, preferably in a concentration of about 150 mM.

In preferred embodiments, the pharmaceutical composition further comprises a sugar, preferably a disaccharide, more preferably sucrose. In embodiments, the concentration of the sugar comprised in the composition is in a range from about 5 mM to about 300 mM, preferably about 14 mM.

In preferred embodiments, the pharmaceutical composition comprises a buffering agent, preferably selected from Tris, HEPES, NaPO4 or combinations thereof. In preferred embodiments, the buffering agent is in a concentration ranging from about 0.1 mM to about 100 mM. In embodiments, the buffering agent is NaPO4, preferably in a concentration of about 1 mM.

In preferred embodiments, the composition has a pH in a range of about pH 7.0 to about pH 8.0. In preferred embodiments, the composition has a pH of about pH 7.4.

In preferred embodiments, the composition has an osmolality of about 250 mOsmol/kg to about 450 mOsmol/kg, preferably of about 335 mOsmol/kg.

In preferred embodiments, the pharmaceutical composition is free of virus particles e.g. attenuated viruses or virus fragments.

In preferred embodiments, the pharmaceutical composition does not comprise and additionally added adjuvant.

The term “adjuvant” is for example intended to refer to a pharmacological and/or immunological agent that may modify, e.g. enhance, the effect of other agents or that may be suitable to support administration and delivery of the composition. The term “adjuvant” refers to a broad spectrum of substances. Typically, these substances are able to increase the immunogenicity of antigens. For example, adjuvants may be recognized by the innate immune systems and, e.g., may elicit an innate immune response (that is, a non-specific immune response). “Adjuvants” typically do not elicit an adaptive immune response.

In preferred embodiments, the pharmaceutical composition of the invention (e.g. component B) is liquid. Accordingly, the pharmaceutical composition may be provided as a liquid composition, e.g. an aqueous formulation.

In other embodiments, the pharmaceutical composition of the invention (e.g. component B) is frozen. Accordingly, the pharmaceutical composition may be provided as a frozen composition, e.g. frozen at temperatures ranging from −80° C. to 0° C., preferably frozen at −80° C., −60° C., −40° C. or −20° C.

In other embodiments, the pharmaceutical composition of the invention (e.g. component B) is lyophilized or spray(freeze) dried.

Accordingly, the pharmaceutical composition may be provided as a lyophilized composition (e.g. wherein lyophilization is performed according to WO2016/165831 or WO2011/069586) or as a spray-freeze dried composition or as a spray dried composition (e.g. wherein spray-freeze drying or spray drying is performed according to WO2016/184575 or WO2016/184576). Accordingly, in that context the disclosures of WO2016/165831, WO2011/069586, WO2016/184575, and WO2016/184576 are incorporated herewith by reference.

In embodiments where the pharmaceutical composition is provided as a lyophilized or spray-freeze dried or spray dried composition, the kit or kit of parts may suitably comprise a buffer for re-constitution of lyophilized or spray-freeze dried or spray dried composition.

In some embodiments, the pharmaceutical composition is provided as a concentrated multidose composition, which requires a dilution before administration via the syringe of component A.

Accordingly, the kit or kit of parts additionally comprises a buffer for re-constitution and/or dilution of the pharmaceutical composition. Preferably, the dilution buffer or re-constitution buffer is provided in a separate container or vial.

In preferred embodiments, the buffer for re-constitution and/or dilution is a sterile buffer. In preferred embodiments, the buffer comprises a salt, preferably NaCL, optionally in a concentration of about 0.9%.

In preferred embodiments, the buffer for re-constitution and/or dilution may additionally comprise a preservative, e.g. a microbial preservative.

In embodiments, the kit or kit of parts is configured for a multi-dose administration, wherein the kit comprises (A) a syringe for injection as defined herein, (B) a pharmaceutical composition comprising RNA, preferably RNA formulated in lipid-based carriers comprising more than one dose, optionally in a higher concentration or in lyophilized or spray-freeze dried or spray dried form, and, optionally (C) a buffer for re-constitution and/or dilution.

In preferred embodiments, the kit or kit of parts is configured for a multi-dose administration.

In preferred embodiments, the pharmaceutical composition comprising RNA is a vaccine.

In preferred embodiments, the vaccine comprises the pharmaceutical composition comprising RNA as defined herein, wherein the RNA encodes at least one antigenic peptide or protein selected from or derived from a pathogen, preferably as defined in the context of the first aspect. Such pathogens may be bacterial, viral, or protozoological (multicellular) pathogenic organisms. Preferably, the pathogen evokes an immunological reaction or an infection in a subject, in particular a mammalian subject, preferably a human subject.

In embodiments, the vaccine is against a pathogen, for example against a virus, against a bacterium, or against a protozoan.

In embodiments, the vaccine is against at least one pathogen selected from List 1.

In embodiments, the vaccine is against a virus.

In preferred embodiments, the vaccine is against a Coronavirus, more preferably against a SARS-CoV-2 coronavirus.

In particularly preferred embodiments, the composition or vaccine comprises an RNA encoding an antigen or epitope selected or derived from a SARS-CoV-2 virus, wherein the RNA comprises or consists of a nucleic acid sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 5 to 10 or a fragment or variant of that sequence. Preferably, the RNA encoding the antigen or epitope selected or derived from a SARS-CoV-2 virus does not comprise chemically modified nucleotides. The RNA encoding the antigen or epitope selected or derived from a SARS-CoV-2 virus is encapsulated in a lipid-based carrier, preferably LNPs, comprising
(i) at least one cationic lipid, preferably a lipid of formula (III) of WO2018/078053A1, more preferably lipid III-3;
(ii) at least one neutral lipid, preferably 1,2-distearoyl-sn-glycero-3-phosphocholine (DSPC);
(iii) at least one steroid or steroid analogue, preferably 1,2-distearoyl-sn-glycero-3-phosphocholine (DSPC); and
(iv) at least one aggregation reducing lipid, preferably a PEG-conjugated lipid derived from formula (IVa); and wherein (i) to (iv) are in a molar ratio of about 47.4% cationic lipid, 10% neutral lipid, 40.9% steroid or steroid analogue, and 1.7% aggregation reducing lipid. Preferably, the concentration of the RNA in the vaccine ranges from about 0.1 μg/ml to about 500 μg/ml and the concentration of lipid in the Coronavirus vaccine ranges from about 2.5 μg/ml to about 12.5 mg/ml. The vaccine is stable for at least 2 weeks after storage as a liquid at temperatures of about 5° C. The N/P ratio of the lipid-based carriers to the RNA in the SARS-CoV-2 vaccine is in a range from about 1 to about 10, preferably in a range from about 5 to about 7, more preferably about 6.

The composition or vaccine typically comprises a safe and effective amount of the RNA as defined herein. As used herein, “safe and effective amount” means an amount of the RNA that significantly induces a positive modification of a disease or disorder related to an infection with a pathogen (e.g. a virus, a bacterium, a protozoan) as specified herein. At the same time, a “safe and effective amount” is small enough to avoid serious side-effects. In relation to the pharmaceutical composition or vaccine, the expression “safe and effective amount” may preferably mean an amount of the composition or vaccine that is suitable for stimulating the adaptive immune system against a pathogen as specified herein in such a manner that no excessive or damaging immune reactions are achieved.

A “safe and effective amount” of the composition or vaccine as defined herein will vary in connection with the particular condition to be treated and also with the age and physical condition of the patient to be treated, the severity of the condition, the duration of the treatment, the nature of the accompanying therapy, of the particular pharmaceutically acceptable carrier used, and similar factors, within the knowledge and experience of the skilled person. Moreover, the “safe and effective amount” of the composition or vaccine may depend from application/delivery route (intradermal, intramuscular, intranasal), and/or complexation/formulation. Moreover, the “safe and effective amount” of the composition or vaccine may depend from the physical condition of the treated subject (infant, pregnant women, immunocompromised human subject etc.).

In embodiments, the composition/vaccine is preferably administered locally. Accordingly, the composition/vaccine is for local administration.

Routes for local administration in general include, for example, topical administration routes but also intradermal, transdermal, subcutaneous, or intramuscular injections or intralesional, intracranial, intrapulmonal, intracardial, intraarticular and sublingual injections. More preferably, the composition/vaccine may be administered by an intradermal, subcutaneous, or intramuscular route, preferably by injection. Preferred in the context of the invention is intramuscular injection. The suitable amount of the composition/vaccine to be administered can be determined by routine experiments, e.g. by using animal models. Such models include, without implying any limitation, rabbit, sheep, mouse, rat, dog and non-human primate models. Preferred unit dose forms for injection include sterile solutions of water, physiological saline or mixtures thereof. The pH should be adjusted to about 7.4.

The composition/vaccine may be used according to the invention for human medical purposes and also for veterinary medical purposes (mammals, vertebrates, or avian species).

In preferred embodiments, the pharmaceutical composition or vaccine is stable for at least 30 minutes, 1 hour, 2 hours, 6 hours, preferably for at least about 6 hours in the a syringe of component A.

In preferred embodiments, the pharmaceutical composition or vaccine is stable for at least 30 minutes to about 6 months in the a syringe of component A. In preferred embodiments, the pharmaceutical composition or vaccine is stable for at least 6 hours, 1 day, 2 days, 3 days, 4 days, 5 days, 6 days, 7 days, 2 weeks, 3 weeks, 4 weeks, 1 month, 2 months, 3 months, 4 months, 5 months, or 6 months in the a syringe of component A.

In embodiments, the composition or vaccine is stable in the syringe of component A at a storage temperature in a range from about −80° C. to about 30° C., preferably in a range from about −20° C. to about 30° C., more preferably in a range from about 5° C. to about 25° C. In specific embodiments, the composition or vaccine comprising RNA formulated in lipid-based carriers is stable at a temperature of about −80° C., about −60° C., about −40° C., about −20° C., about −10° C., abbot 0° C., about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C.

In preferred embodiments, the pharmaceutical composition or vaccine is stable in the syringe of component A at a temperature of about 5° C. to about 25° C. In embodiments, the pharmaceutical composition or vaccine is stable in the a syringe of component A at a temperature of about 5° C., 6° C., 7° C., 8° C., 9° C., 10° C., 15° C., 20° C., or 25° C.

According to preferred embodiments, the pharmaceutical composition or vaccine is stable in the syringe of component A at a temperature of about 5° C. to about 25° C. for at least 6 hours.

Advantageously, the physiochemical properties of the RNA and/or the lipid-based carriers are stable in the presence of the a syringe of component A.

In preferred embodiments, after exposure (e.g. storage) to the syringe of component A, the integrity of the RNA decreases less than about 30%, preferably less than about 20%, more preferably less than about 10%. RNA integrity is suitably determined using analytical HPLC, preferably analytical RP-HPLC. In preferred embodiments, after exposure to the syringe of component A, the RNA has an RNA integrity ranging from about 40% to about 100%. RNA integrity is suitably determined using analytical HPLC, preferably analytical RP-HPLC.

In preferred embodiments, after exposure (e.g. storage) to the syringe of component A, the amount of free RNA does not increase by more than 20%, preferably by not more than 10%, more preferably by not more than 5%. In embodiments, after exposure to the syringe of component A, the amount of free RNA in the composition or vaccine ranges from about 30% to about 0%. Free RNA is suitably determined using a RiboGreen assay.

In embodiments, after exposure (e.g. storage) to the syringe of component A, the percentage of RNA encapsulation does not decrease by more than 20%, preferably by not more than 10%. In embodiments, after exposure to the syringe of component A, the percentage of RNA encapsulation ranges from about 60% to about 100%. RNA encapsulation is suitably determined using a RiboGreen assay.

In embodiments, after exposure (e.g. storage) to the syringe of component A, the PDI value does not increase by more than a value of about 0.2, preferably by not more than a value of about 0.1. In embodiments, after exposure to the syringe of component A, the PDI value ranges from about 0.4 to about 0.0. PDI is suitably determined using DLS.

In embodiments, after exposure (e.g. storage) to the syringe of component A, the Z-average size of the lipid based carriers does not increase by more than 20%, preferably by not more than 10%. In embodiments, after exposure to the syringe of component A, the Z-average size of the lipid based carriers ranges from about 50 nm to about 150 nm. Z-average size is suitably determined using DLS.

In embodiments, after exposure (e.g. storage) to the syringe of component A, the number of sub visible particles ≥2 μm (#per ml) in the composition or vaccine is not increased by more than 20%, preferably by not more than 10%.

In embodiments, after exposure (e.g. storage) to the syringe of component A, the potency of the composition or vaccine decreases less than about 30%, preferably less than 20%, more preferably less than 10%. In embodiments, potency is the expression of the encoded peptide or protein upon administration of the composition to a cell, and/or the induction of specific antibody titers upon administration of the composition to a cell, and/or the induction of neutralizing antibody titers upon administration of the composition to a cell, and/or the induction of antigen-specific T-cell responses upon administration of the composition to a cell.

In embodiments, after exposure (e.g. storage) to the syringe of component A, the reactogenicity of the composition or vaccine does not increase by more than 20%, preferably by not more than 10%. In embodiments, reactogenicity may be the induction of (undesired) innate immune responses upon administration of the composition to a cell (e.g. cytokine induction).

In embodiments, after exposure (e.g. storage) to the syringe of component A, the syringe of produces less than about 20%, 19%, 18%, 17%, 16%, 15%, 14%, 13%, 12%, 11%, 10%, 9%, 8%, 7%, 6%, 5%, 4%, 3% or 2% RNA agglomeration in the composition or vaccine.

In embodiments, the composition/vaccine elicits an adaptive immune response against at least one pathogen when administered with the syringe of component A to a cell or a subject, wherein the at least one pathogen may be selected from a bacterium, a protozoan, or a virus, for example from a pathogen provided in List 1.

In preferred embodiments the composition/vaccine elicits an adaptive immune response against a Coronavirus (e.g. SARS-CoV-2) when administered with the syringe of component A to a cell or a subject. In preferred embodiments, upon storage of the composition/vaccine in the syringe of component A, said adaptive immune response does not decrease by more than 20%, preferably by not more than 10%.

In embodiments, administration of a therapeutically effective amount of the composition/vaccine elicits neutralizing antibody titers against at least one pathogen, wherein the at least one pathogen may be selected from a bacterium, a protozoan, or a virus, for example from a pathogen provided in List 1. In preferred embodiments the composition/vaccine elicits neutralizing antibody titers against a Coronavirus (e.g. SARS-CoV-2) when administered with the syringe of component A to a cell or a subject. In preferred embodiments, upon storage of the composition/vaccine in the syringe of component A, said neutralizing antibody titers do not decrease by more than 20%, preferably by not more than 10%.

In embodiments, detectable levels of the respective antigen (e.g. SARS-CoV-2 antigen) are in the subject at about 1 to about 72 hours post administration of the composition/vaccine. In embodiments, upon storage of the composition/vaccine in the syringe of component A, detectable levels of the respective antigen are produced in the subject at about 1 to about 72 hours post administration.

In embodiments, the neutralizing antibody titer that is induced upon administration with the syringe of component A of the composition/vaccine to a subject is at least 100 neutralizing units per millilitre (NU/mL), at least 500 NU/mL, or at least 1000 NU/mL. In embodiments, upon storage of the composition/vaccine in the syringe of component A, the neutralizing antibody titer induced upon administration of the composition/vaccine to a subject is at least 100 neutralizing units per milliliter (NU/mL), at least 500 NU/mL, or at least 1000 NU/mL.

In some embodiments, a neutralizing antibody titer of at least 100 NU/ml, at least 500 NU/ml, or at least 1000 NU/ml is produced in the serum of the subject at about 72 hours post administration of the composition/vaccine with the syringe of component A. In embodiments, upon storage of the composition/vaccine in the syringe of component A, a neutralizing antibody titer of at least 100 NU/ml, at least 500 NU/ml, or at least 1000 NU/ml is produced in the serum of the subject at about 72 hours post administration of the composition/vaccine.

In some embodiments, the neutralizing antibody titer is sufficient to reduce infection with a pathogen by at least 50% relative to a neutralizing antibody titer of an unvaccinated control subject or relative to a neutralizing antibody titer of a subject vaccinated with a live attenuated viral vaccine, an inactivated viral vaccine, or a protein sub unit viral vaccine. In embodiments, upon storage of the composition/vaccine in the syringe of component A, the neutralizing antibody titer is sufficient to reduce infection with a pathogen by at least 50% relative to a neutralizing antibody titer of an unvaccinated control subject or relative to a neutralizing antibody titer of a subject vaccinated with a live attenuated viral vaccine, an inactivated viral vaccine, or a protein sub unit viral vaccine.

In some embodiments, the neutralizing antibody titer and/or the T cell immune response is sufficient to reduce the rate of asymptomatic pathogen (e.g. a Coronavirus) infection relative to the neutralizing antibody titer of unvaccinated control subjects. In embodiments, upon storage of the composition/vaccine in the syringe of component A, the neutralizing antibody titer and/or the T cell immune response is sufficient to reduce the rate of asymptomatic pathogen (e.g. a Coronavirus) infection relative to the neutralizing antibody titer of unvaccinated control subjects.

In some embodiments, the neutralizing antibody titer and/or a T cell immune response is sufficient to prevent viral latency in the subject. In embodiments, upon storage of the composition/vaccine in the syringe of component A, the neutralizing antibody titer and/or a T cell immune response is sufficient to prevent viral latency in the subject.

In preferred embodiments, administration of a therapeutically effective amount of the composition/vaccine with the syringe of component A to a subject induces a T cell immune response against a pathogen (e.g. a Coronavirus) in the subject, preferably wherein the T cell immune response comprises a CD4+ T cell immune response and/or a CD8+ T cell immune response. In embodiments, upon storage of the composition/vaccine in the syringe of component A, administration of a therapeutically effective amount of the composition/vaccine to a subject induces a T cell immune response against a pathogen (e.g. a Coronavirus) in the subject, preferably wherein the T cell immune response comprises a CD4+ T cell immune response and/or a CD8+ T cell immune response

In some embodiments, the neutralizing antibody titer is sufficient to block fusion of a pathogen (e.g. a Coronavirus) with epithelial cells of the subject. In embodiments, upon storage of the composition/vaccine in the syringe of component A, the neutralizing antibody titer is sufficient to block fusion of a pathogen (e.g. a Coronavirus) with epithelial cells of the subject

In some embodiments, the neutralizing antibody titer is induced within 20 days following a single 1 ug-100 ug dose of the composition/vaccine, or within 40 days following a second 1 ug-100 μg dose of the composition/vaccine, wherein, preferably, the dose relates to the amount of the RNA. In preferred embodiments, a dose comprises less that about 100 μg, preferably less than about 50 μg, more preferably less than about 20 μg, even more preferably less than about 10 μg, wherein preferably, the dose relates to the amount of the RNA.

In preferred embodiments, the composition/vaccine elicits antigen-specific immune responses in a subject that has an age of about 5 years old or younger. In preferred embodiments, upon storage of the composition/vaccine in the syringe of component A, the composition/vaccine elicits antigen-specific immune responses in a subject that has an age of about 5 years old or younger. Accordingly, the compositions/vaccines of the invention are particularly suitable for infants.

In preferred embodiments, the composition/vaccine elicits antigen-specific immune responses in a subject that has an age of about 60 years old or older. In preferred embodiments, upon storage of the composition/vaccine in the syringe of component A, the composition/vaccine elicits antigen-specific immune responses in a subject that has an age of about 60 years old or older. Accordingly, the compositions/vaccines of the invention are particularly suitable for the elderly.

### A Pre-Filled Syringe Containing a Pharmaceutical Composition Comprising RNA

In a second aspect, the present invention provides a pre-filled syringe for injection containing a pharmaceutical composition comprising RNA.

It has to be noted that features and embodiments that are described in the context of the pharmaceutical composition or vaccine of the kit or kit of parts of the first aspect (also referred to as “component B” in the first aspect) may also be applicable to the pharmaceutical composition that is contained in the pre-filled syringe for injection as further specified herein. Likewise, features and embodiments referring to the pharmaceutical composition that is contained in the pre-filled syringe for injection that are described in the context of the second aspect may also be applicable to the pharmaceutical composition or vaccine of the kit or kit of parts of the first aspect.

Further, it has to be noted that features and embodiments that are described in the context of the syringe for injection of the kit or kit of parts of the first aspect (also referred to as “component A” in the first aspect) may also be applicable to the pre-filled syringe for injection as further specified herein (e.g. features describing structural or functional features of suitable syringes). Likewise, features and embodiments referring to the pre-filled syringe for injection that are described in the context of the second aspect may also be applicable to the syringe for injection of the kit or kit of parts of the first aspect.

Particularly preferred features and embodiments of the pre-filled syringe are provided in form of an item list at the end of that section.

In the context of the invention, a pre-filled syringe has to be understood as a syringe that contains a pharmaceutical composition, wherein the pharmaceutical composition has been loaded into the syringe by a manufacturer. Accordingly, a pre-filled syringe in the context of the invention is not a standard disposable syringe that contains a pharmaceutical composition and that has been loaded by withdrawing a certain dose from e.g. a multidose vial.

Accordingly, in preferred embodiments, the pre-filled syringe has been loaded with a pharmaceutical composition comprising RNA by a manufacturer. Preferably, the loading process to obtain the pre-filled syringe has been performed under aseptic conditions, preferably in an automated manner (e.g. by a machine or device) and not by manually handling (e.g. by a medical doctor).

Typically, a pre-filled syringe comprises essentially one dose of the pharmaceutical composition to be applied to a subject.

In a particularly preferred embodiments of the second aspect, the pre-filled syringe for injection contains a pharmaceutical composition comprising RNA, wherein preferably less than 20% of the RNA of the contained pharmaceutical composition is agglomerated, wherein the syringe used for obtaining the pre-filled syringe is characterized by at least one of the following features
(i) the inner surface of the syringe barrel and/or the syringe plunger stopper is essentially free of silicone oils;
(ii) the syringe produces less than about 20% RNA agglomeration when incubated with an aqueous test formulation comprising RNA encapsulated in LNPs for 6 hours at 20° C.;
(iii) the syringe produces less than about 20% RNA agglomeration when incubated with the pharmaceutical composition comprising RNA for 6 hours at 20° C.; or
(iv) the syringe produces less than 10 mAU*min of extractable compounds in 20 μl of a 2-Propanol extract as determined by analytical (RP)HPLC.

In particularly preferred embodiments, the syringe used for obtaining the pre-filled syringe is characterized by features (i), (ii), (iii), and (iv). In particularly preferred embodiments, the syringe used for obtaining the pre-filled syringe is characterized by features (i), (ii), and (iii). In particularly preferred embodiments, the syringe used for obtaining the pre-filled syringe is characterized by features (i), (ii), and (iv). In particularly preferred embodiments, the syringe used for obtaining the pre-filled syringe is characterized by features (i), (iii), and (iv). In particularly preferred embodiments, the syringe used for obtaining the pre-filled syringe is characterized by features (i), and (ii). In particularly preferred embodiments, the syringe used for obtaining the pre-filled syringe is characterized by features (i), and (iii). In particularly preferred embodiments, the syringe used for obtaining the pre-filled syringe is characterized by features (i), and (iv). In preferred embodiments, the syringe used for obtaining the pre-filled syringe is characterized by features (ii), (iii), and (iv). In preferred embodiments, the syringe used for obtaining the pre-filled syringe is characterized by features (ii), (iii). In preferred embodiments, the syringe used for obtaining the pre-filled syringe is characterized by features (ii), (iv). In preferred embodiments, the syringe used for obtaining the pre-filled syringe is characterized by features (iii), and (iv).

In particularly preferred embodiments, the pre-filled syringe contains a pharmaceutical composition comprising RNA, wherein the syringe used for obtaining the pre-filled syringe is characterized by that (i) the inner surface of the syringe barrel and/or the syringe plunger stopper is essentially free of silicone oils. Suitably, the pre-filled syringe is free of silicone oils.

In preferred embodiments, the RNA is formulated in lipid-based carriers (as further described herein in detail e.g. in the context of the first aspect). Preferably, the RNA is a single stranded RNA. Preferably, the RNA is not an antisense RNA or an siRNA. Preferably, the contained composition does not comprise a protein based or peptide based medicament.

Preferably, the RNA is a long chain RNA, suitably wherein the long-chain RNA is larger than about 100 nucleotides (e.g. about 100 to about 10,000 nucleotides).

In preferred embodiments, the RNA is a single stranded or long chain RNA formulated in lipid-based carriers (as further specified in the first aspect).

In preferred embodiments, the RNA is a single stranded or long chain RNA formulated in lipid-based carriers (as further specified in the first aspect) and the contained composition does not comprise a peptide or protein.

Suitably, the less than 20% of the RNA of the contained pharmaceutical composition is agglomerated.

In preferred embodiments, the pre-filled syringe contains a pharmaceutical composition comprising RNA, wherein the syringe used for obtaining the pre-filled syringe is characterized by that (i) the inner surface of the syringe barrel and/or the syringe plunger stopper is essentially free of silicone oils, wherein the syringe used for obtaining the pre-filled syringe is further characterized by at least one of the

following features
(ii) the syringe produces less than about 20% RNA agglomeration when incubated with an aqueous test formulation comprising RNA encapsulated in LNPs for 6 hours at 20° C.;
(iii) the syringe produces less than about 20% RNA agglomeration when incubated with the pharmaceutical composition of component B for 6 hours at 20° C.;
(iv) the syringe produces less than 10 mAU*min of extractable compounds in 20 μl of a 2-Propanol extract as determined by analytical (RP)HPLC; or
(v) a combination of any of (ii) to (iv);
   preferably, wherein the RNA of component B is formulated in lipid-based carriers and/or wherein the RNA of component B is a single stranded or long chain RNA.

Whenever certain features and embodiments are provided herein that refer to a “syringe used for obtaining the pre-filled syringe”, these features of course also relate to the pre-filled syringe as such.

Accordingly, the pre-filled syringe is a silicone-oil free syringe. Suitably, the pre-filled syringe is further characterized by features (ii), (iii), (iv) and/or (v).

In preferred embodiments, the syringe used for obtaining the pre-filled syringe is configured for (sterile) pre-filling of a medicament.

Particularly preferred syringes may be selected from the materials or syringe systems (or combinations thereof) as described e.g. in the example section, preferably silicone-oil free syringe S1 (SOF-S1), silicone-oil free syringe S2 (SOF-S2), or silicone-oil free syringe S3 (SOF-S3). Such syringes may comprise a glass barrel or a cyclic olefin copolymer barrel (see also first aspect and Table A and Table B) In preferred embodiments, the pre-filled syringe comprises more than one compartment. For example, the syringe according to the invention may comprise one compartment where the pharmaceutical composition is contained (e.g. as a liquid composition or lyophilized composition), and additionally, one compartment where a buffer for dilution and/or re-constitution as defined herein is contained. Such an embodiment is particularly important in the case where the pharmaceutical composition is provided in a lyophilized or spray-dried or spray-freeze dried form, or where the pharmaceutical composition is provided in a higher concentration (e.g. a higher concentration of RNA formulated in lipid-based carriers) than the concentration needed for administration.

In preferred embodiments, the syringe used for obtaining the pre-filled syringe is configured for use in a freezing step and/or a lyophilization step.

In preferred embodiments, the syringe or the syringe barrel is of a cryo-resistant material.

Such embodiments are particularly important in the case where the pre-filled syringe contains a lyophilized pharmaceutical composition, and where the pre-filled syringe containing the lyophilized pharmaceutical composition is obtained by lyophilizing a liquid pharmaceutical composition in said syringe. Suitably, the syringe is of a cryo-resistant material that allows lyophilization (e.g. glass). In other embodiments, the syringe is of a cryo-resistant material that allows a deep freezing temperature, e.g. −80° C., −60° C., −20° C. This is particularly important in embodiments where the pre-filled syringe containing the pharmaceutical composition comprising RNA formulated in lipid-based carriers has to be stored at −80° C., −60° C., or −20° C.

In preferred embodiments, the pharmaceutical composition comprising RNA (contained in the syringe) is essentially free of silicone oils, lubricants oils or lubricants that are soluble in an organic solvent.

Accordingly, the pre-filled syringe is configured so that no silicone oils, lubricants oils, or lubricants that are soluble in an organic solvent is leaching into the pharmaceutical composition that is contained in the syringe. Without whishing to be bound to theory, silicone oils (or lubricant oils or lubricants that are soluble in an organic solvent) may cause RNA aggregation, therefore it is desirable that the pharmaceutical composition (contained in the syringe) is essentially free of silicone oils, lubricants oils or lubricants that are soluble in an organic solvent (e.g., 2-Propanol).

In preferred embodiments, the pharmaceutical composition in the pre-filled syringe comprises about 1 μg to about 200 μg of RNA, preferably about 1 μg to about 100 μg, more preferably about 1 μg to about 25 μg of RNA. In specific embodiments, the pharmaceutical composition in the pre-filled syringe comprises about 12 μg, about 30 μg or about 100 μg. In preferred embodiments, the pre-filled syringe comprises less than about 30 μg RNA, preferably less than about 20 μg RNA, e.g. 29 μg, 28 μg, 27 μg, 26 μg, 25 μg, 24 μg, 23 μg, 22 μg, 21 μg, 20 μg, 19 μg, 18 μg, 17 μg, 16 μg, 15 μg, 14 μg, 13 μg, 12 μg, 11 μg, 10 μg, 9 μg, 8 μg, 7 μg, 6 μg, 5 μg, 4 μg, 2 μg, 2 μg, 1 μg RNA.

In preferred embodiments, the pharmaceutical composition in the pre-filled syringe comprises RNA formulated in lipid-based carriers.

In preferred embodiments, the pre-filled syringe is suitable for use as a medicament, e.g. a vaccine.

In preferred embodiments, the pre-filled syringe, in particular the pharmaceutical composition or vaccine contained in the syringe, is stable for at least 30 minutes, 1 hour, 2 hours, 6 hours, preferably for at least about 6 hours.

In preferred embodiments, the pre-filled syringe, in particular the pharmaceutical composition or vaccine contained in the syringe, is stable for at least 30 minutes to about 6 months. In preferred embodiments, the pre-filled syringe, in particular the pharmaceutical composition or vaccine contained in the syringe, is stable for at least 6 hours, 1 day, 2 days, 3 days, 4 days, 5 days, 6 days, 7 days, 2 weeks, 3 weeks, 4 weeks, 1 month, 2 months, 3 months, 4 months, 5 months, or 6 months.

In embodiments, the pre-filled syringe, in particular the pharmaceutical composition or vaccine contained in the syringe is stable at a storage temperature in a range from about −80° C. to about 30° C., preferably in a range from about −20° C. to about 30° C., more preferably in a range from about 5° C. to about 25° C. In specific embodiments, the pre-filled syringe, in particular the pharmaceutical composition or vaccine contained in the syringe, is stable at a temperature of about −80° C., about −60° C., about −40° C., about −20° C., about −10° C., abbot 0° C., about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C. Preferred examples of a storage temperature in the context of the invention are −80° C., −20° C., 5° C., or room temperature (about 20° C.).

In preferred embodiments, the pre-filled syringe, in particular the pharmaceutical composition or vaccine contained in the syringe, is stable at a temperature of about 5° C. to about 25° C. In embodiments, the pre-filled syringe, in particular the pharmaceutical composition or vaccine contained in the syringe, is stable at a temperature of about 5° C., 6° C., 7° C., 8° C., 9° C., 10° C., 15° C., 20° C., or 25° C.

According to preferred embodiments, the pre-filled syringe, in particular the pharmaceutical composition or vaccine contained in the syringe, is stable at a temperature of about 5° C. to about 25° C. for at least 6 hours.

Advantageously, the physiochemical properties of the RNA and/or the lipid-based carriers contained in the syringe are stable over a time of storage (e.g. integrity of the RNA, amount of free RNA, percentage of RNA encapsulation, PDI value, Z-average size of the lipid based carriers, number of sub visible particles, potency of the composition or vaccine, reactogenicity of the composition or vaccine, RNA agglomeration, immune response, etc.).

In preferred embodiments, the integrity of the RNA in the pre-filled syringe decreases less than about 30%, preferably less than about 20%, more preferably less than about 10% over a time of storage.

In preferred embodiments, the amount of free RNA in the pre-filled syringe does not increase by more than 20%, preferably by not more than 10%, more preferably by not more than 5% over a time of storage.

In preferred embodiments, the percentage of RNA encapsulation in the pre-filled syringe does not decrease by more than 20%, preferably by not more than 10% over a time of storage.

In preferred embodiments, the PDI value of the composition/vaccine in the pre-filled syringe does not increase by more than a value of about 0.2, preferably by not more than a value of about 0.1 over a time of storage.

In preferred embodiments, the Z-average size of the lipid based carriers in the pre-filled syringe does not increase by more than 20%, preferably by not more than 10% over a time of storage

In preferred embodiments, the number of sub visible particles ≥2 μm (#per ml) in the composition or vaccine in the pre-filled syringe is not increased by more than 20%, preferably by not more than 10% over a time of storage.

In preferred embodiments, potency of the composition or vaccine in the pre-filled syringe decreases less than about 30%, preferably less than 20%, more preferably less than 10% over a time of storage.

In preferred embodiments, the reactogenicity of the composition or vaccine in the pre-filled syringe does not increase by more than 20%, preferably by not more than 10% over a time of storage.

In preferred embodiments, the composition or vaccine in the pre-filled syringe comprises less than about 20%, 19%, 18%, 17%, 16%, 15%, 14%, 13%, 12%, 11%, 10%, 9%, 8%, 7%, 6%, 5%, 4%, 3% or 2% RNA agglomeration over a time of storage.

In embodiments, the composition/vaccine in the pre-filled syringe elicits an adaptive immune response against at least one pathogen when administered to a cell or a subject, wherein the at least one pathogen may be selected from a bacterium, a protozoan, or a virus, for example from a pathogen provided in List 1.

In preferred embodiments the composition/vaccine in the pre-filled syringe elicits an adaptive immune response against a Coronavirus (e.g. SARS-CoV-2) when administered to a cell or a subject. In preferred embodiments, said adaptive immune response does not decrease by more than 20%, preferably by not more than 10% over a time of storage.

In embodiments, administration of a therapeutically effective amount of the composition/vaccine in the pre-filled syringe elicits neutralizing antibody titers against at least one pathogen, wherein the at least one pathogen may be selected from a bacterium, a protozoan, or a virus, for example from a pathogen provided in List 1. In preferred embodiments the composition/vaccine in the pre-filled syringe elicits neutralizing antibody titers against a Coronavirus (e.g. SARS-CoV-2) when administered to a cell or a subject. In preferred embodiments, said neutralizing antibody titers do not decrease by more than 20%, preferably by not more than 10% over a time of storage.

In embodiments, detectable levels of the respective antigen (e.g. SARS-CoV-2 antigen) are in the subject at about 1 to about 72 hours post administration of the composition/vaccine in the pre-filled syringe. In embodiments, detectable levels of the respective antigen are produced in the subject at about 1 to about 72 hours post administration over a time of storage.

In embodiments, the neutralizing antibody titer that is induced upon administration of the composition/vaccine to a subject is at least 100 neutralizing units per millilitre (NU/mL), at least 500 NU/mL, or at least 1000 NU/mL. In embodiments, the neutralizing antibody titer induced upon administration of the composition/vaccine to a subject is at least 100 neutralizing units per milliliter (NU/mL), at least 500 NU/mL, or at least 1000 NU/mL over a time of storage.

As used herein, the term “over a time of storage” has to be understood as a storage under a condition as defined herein. For example, storage may be for at least 30 minutes, 1 hour, 2 hours, 6 hours, 1 days, 2 days, 1 week, 2 weeks, 3 weeks, 1 month, 2 months, 3 months, 4 months, 5 months, or 6 months at about 5° C. Alternatively or additionally, storage may be for at least 30 minutes, 1 hour, 2 hours, 6 hours, 1 day, 2 days, 1 week, 2 weeks, 3 weeks, or 4 weeks at about 20° C.

In the following, particularly preferred embodiments of the pre-filled syringe for injection containing a pharmaceutical composition comprising RNA formulated in lipid-based carriers are listed in form of an item list.

### Item List: Pre-Filled Syringe Containing a Pharmaceutical Composition Comprising RNA

Item 1a: A pre-filled syringe for injection containing a pharmaceutical composition comprising RNA, optionally wherein less than 20% of the RNA of the contained pharmaceutical composition is agglomerated upon storage in the syringe, wherein the syringe or the syringe used for obtaining the pre-filled syringe is characterized by at least one of the following features
(i) the inner surface of the syringe barrel and/or the syringe plunger stopper is essentially free of silicone oils;
(ii) the syringe produces less than about 20% RNA agglomeration when incubated with an aqueous test formulation comprising RNA encapsulated in LNPs for 6 hours at 20° C.;
(iii) the syringe produces less than about 20% RNA agglomeration when incubated with the pharmaceutical composition comprising RNA for 6 hours at 20° C.; or
(iv) the syringe produces less than 10 mAU*min of extractable compounds in 20 μl of a 2-Propanol extract as determined by analytical RP-HPLC.
Item 1b: A pre-filled syringe for injection containing a pharmaceutical composition comprising RNA, wherein the syringe or the syringe used for obtaining the pre-filled syringe is characterized by that (i) the inner surface of the syringe barrel and/or the syringe plunger stopper is essentially free of silicone oils.
Item 2a: The pre-filled syringe of item 1a or 1b, wherein the RNA is formulated in lipid-based carriers Item 2b: The pre-filled syringe of any one of the preceding items, wherein the RNA is a single stranded RNA or a long chain RNA Item 2c: The pre-filled syringe of any one of the preceding items, wherein the RNA is not an antisense RNA or an siRNA.
Item 2d: The pre-filled syringe of any one of the preceding items, wherein the contained pharmaceutical composition does not comprise a peptide or protein medicament.
Item 2e: The pre-filled syringe of any one of the preceding items, wherein less than 20% of the RNA of the contained pharmaceutical composition is agglomerated.
Item 2f: The pre-filled syringe of items 1b to 2d, wherein the syringe or the syringe used for obtaining the pre-filled syringe is characterized by at least one of the following features
(ii) the syringe produces less than about 20% RNA agglomeration when incubated with an aqueous test formulation comprising RNA encapsulated in LNPs for 6 hours at 20° C.;
(iii) the syringe produces less than about 20% RNA agglomeration when incubated with the pharmaceutical composition of component B for 6 hours at 20° C.;
(iv) the syringe produces less than 10 mAU*min of extractable compounds in 20 μl of a 2-Propanol extract as determined by analytical (RP)HPLC; or
(v) a combination of any of (ii) to (iv).
Item 2g: The pre-filled syringe of any one of the preceding items, wherein the inner surface of the syringe barrel is essentially free of silicone oils, the syringe plunger is essentially free of silicone oils, the syringe plunger stopper is essentially free of silicone oils, the needle adapter is essentially free of silicone oils, and/or the needle hub is essentially free of silicone oils.
Item 3: The pre-filled syringe of any one of the preceding items, wherein the syringe barrel, the syringe plunger, and/or the syringe plunger stopper comprises a material that is suitable for use in a syringe without silicone oil or with a low amount of silicone oil.
Item 4: The pre-filled syringe of any one of the preceding items, wherein the syringe barrel comprises a polymer preferably selected from olefin polymer, cyclic olefin copolymer (COP), polypropylene, polysterene, polyethylene and/or polycarbonate, preferably polypropylene or COP.
Item 5A: The pre-filled syringe of any one of the preceding items, wherein the syringe barrel comprises glass.
Item 5B: The pre-filled syringe of any one of the preceding items, wherein the syringe barrel comprises a glass coating of the inner surface or a silicon dioxide coating of the inner surface.
Item 6: The pre-filled syringe of any one of the preceding items, wherein the syringe plunger stopper comprises a thermoplastic elastomer, a silicone polymer, or a rubber, and/or a coating that reduces the gliding force.
Item 7: The pre-filled syringe of any one of the preceding items, wherein the syringe is configured for intramuscular, intradermal, intratumoral, intravenous, or intraocular injection (e.g. intravitreal injection), preferably intramuscular injection.
Item 8: The pre-filled syringe of any one of the preceding items, wherein the syringe of has a volume of about 100 μl to about 25 ml, preferably 100 μl to about 10 ml, even more preferably 100 μl to 2 ml, still more preferably 100 μl to about 1 ml.
Item 9: The pre-filled syringe of any one of the preceding items, wherein the syringe comprises more than one compartment, preferably wherein one compartment contains the pharmaceutical composition comprising RNA formulated in lipid-based carriers and a compartment contains a buffer for re-constitution and/or dilution.
Item 10: The pre-filled syringe of any one of the preceding items, wherein the syringe barrel or the syringe is configured for use in a freezing step and/or a lyophilization step.
Item 11: The pre-filled syringe of any one of the preceding items, wherein the syringe barrel or the syringe is of a cryo-resistant material.
Item 12: The pre-filled syringe of any one of the preceding items, wherein less than about 20%, 19%, 18%, 17%, 16%, 15%, 14%, 13%, 12%, 11%, 10%, 9%, 8%, 7%, 6%, 5%, 4%, 3% or 2% of the RNA of the pharmaceutical composition is agglomerated (upon a time of storage).
Item 13: The pre-filled syringe of any one of the preceding items, wherein the syringe used for obtaining the pre-filled syringe produces less than about 20%, 19%, 18%, 17%, 16%, 15%, 14%, 13%, 12%, 11%, 10%, 9%, 8%, 7%, 6%, 5%, 4%, 3% or 2% RNA agglomeration when incubated with an aqueous test formulation comprising RNA encapsulated in LNPs for 6 hours at 20° C. or when incubated with the pharmaceutical composition for 6 hours at 20° C.
Item 14: The pre-filled syringe of any one of the preceding items, wherein the RNA agglomeration is measured using analytical (RP)HPLC of RNA isolated from the components of the aqueous test formulation or the pharmaceutical composition.
Item 15A: The pre-filled syringe of item 14, wherein the RNA agglomeration in % is determined based on the proportion of the relative peak area of the tail in the obtained chromatogram, e.g. the obtained HPLC chromatogram.
Item 15B: The pre-filled syringe of item 14 or 15A, wherein analytical (RP)HPLC is performed on an analytical monolithic poly(styrene-divinylbenzene) column.
Item 16: The pre-filled syringe of any one of the preceding items, wherein the syringe used for obtaining the pre-filled syringe produces less than 10 mAU*min, 9 mAU*min, 9 mAU*min, 7 mAU*min, 6 mAU*min, 5 mAU*min of detectable compounds in 20 μl of a 2-Propanol extract as determined by analytical (RP)HPLC (UV 260 nm).
Item 17: The pre-filled syringe of item 16, wherein the analytical (RP)HPLC is performed on a C18 modified analytical HPLC column, preferably a BEH C18 column.
Item 18: The pre-filled syringe of items 16 or 17, wherein the 2-Propanol extract is obtained by three repeated draw/eject cycles using 1 mL 2-Propanol room temperature.
Item 19: The pre-filled syringe of any one of the preceding items, wherein the contained pharmaceutical composition is essentially free of silicone oils, lubricants oils or extractable lubricant oils that are soluble in an organic solvent.
Item 20: The pre-filled syringe of any one of the preceding items, wherein the pharmaceutical composition contained in the pre-filled syringe, is stable at a temperature of about 5° C. to about 25° C. for at least 6 hours.
Item 21: The pre-filled syringe of any one of the preceding items, wherein the concentration of RNA in the pharmaceutical composition contained in the pre-filled syringe is in a range of about 0.1 μg/ml to about 500 μg/ml, preferably in a range of about 0.1 μg/ml to about 100 μg/ml, more preferably in a range of about 1 μg/ml to about 100 μg/ml.
Item 22: The pre-filled syringe of any one of the preceding items, wherein the concentration of RNA in the pharmaceutical composition contained in the pre-filled syringe is lower than 200 μg/ml, preferably lower than at least 100 μg/ml, more preferably lower than 50 μg/ml.
Item 23: The pre-filled syringe of any one of the preceding items, wherein the pharmaceutical composition contained in the pre-filled syringe comprises about 1 μg to about 200 μg of RNA, preferably about 1 μg to about 25 μg of RNA.
Item 24: The pre-filled syringe of any one of the preceding items, wherein the RNA of the pharmaceutical composition contained in the pre-filled syringe has an RNA integrity of at least about 50%, preferably of at least about 60%, more preferably of at least about 70%, most preferably of at least about 80%.
Item 25A: The pre-filled syringe of any one of the preceding items, wherein the RNA of the pharmaceutical composition contained in the pre-filled syringe has an RNA integrity of at least about 50%, preferably of at least about 60%, more preferably of at least about 70%, most preferably of at least about 80% upon storage.
Item 25B: The pre-filled syringe of any one of the preceding items, wherein the syringe used for obtaining the pre-filled syringe produces less than about 20%, 19%, 18%, 17%, 16%, 15%, 14%, 13%, 12%, 11%, 10%, 9%, 8%, 7%, 6%, 5%, 4%, 3% or 2% reduction in RNA integrity (or Delta RNA integrity in %) when incubated with an aqueous test formulation comprising RNA encapsulated in LNPs, preferably for 6 hours at 20° C.
Item 25C: The pre-filled syringe of any one of the preceding items, wherein the syringe produces less than about 20%, 19%, 18%, 17%, 16%, 15%, 14%, 13%, 12%, 11%, 10%, 9%, 8%, 7%, 6%, 5%, 4%, 3% or 2% reduction in RNA integrity when incubated with the pharmaceutical composition, preferably for 6 hours at 20° C.
Item 26: The pre-filled syringe of any one of the preceding items, wherein the pharmaceutical composition contained in the pre-filled syringe comprises less than about 20% free RNA, preferably less than about 15% free RNA, more preferably less than about 10% free RNA.
Item 27: The pre-filled syringe of any one of the preceding items, wherein the RNA is at least about 100 nucleotides in length, preferably at least about 1000 nucleotides in length.
Item 28: The pre-filled syringe of any one of the preceding items, wherein the RNA has a length ranging from about 100 nucleotides to about 10000 nucleotides, preferably ranging from about 500 nucleotides to about 10000 nucleotides, more preferably ranging from about 1000 nucleotides to about 10000 nucleotides.
Item 29a: The pre-filled syringe of any one of the preceding items, wherein the RNA consists of non-modified A, U, G, and C ribonucleotides, and optionally a 5′cap structure.
Item 29b: The pre-filled syringe of items 1 to 28, wherein the RNA of the pharmaceutical composition comprises chemically modified nucleotides preferably selected from pseudouridine (ψ) or N1-methylpseudouridine (ml p).
Item 30: The pre-filled syringe of any one of the preceding items, wherein the is an mRNA.
Item 31: The pre-filled syringe of any one of the preceding items, wherein RNA has a GC content of at least about 55%, preferably at least about 60%.
Item 32: The pre-filled syringe of any one of the preceding items, wherein the RNA comprises a 5′ cap structure, preferably a cap1 structure, more preferably wherein at least 70%, 80%, or 90% of the RNA species comprise a cap1 structure.
Item 33: The pre-filled syringe of any one of the preceding items, wherein the RNA comprises at least one poly(A) sequence, and/or at least one poly(C) sequence, and/or at least one histone stem-loop and/or at least one 5′-UTR and/or at least one 3′-UTR.
Item 34: The pre-filled syringe of any one of the preceding items, wherein the RNA comprises a coding sequence encoding at least one peptide or protein suitable for use in treatment or prevention of a disease, disorder or condition.
Item 35: The pre-filled syringe of item 34, wherein the at least one peptide or protein is selected or derived from an antibody, an intrabody, a receptor, a receptor agonist, a receptor antagonist, a binding protein, a CRISPR-associated endonuclease, a chaperone, a transporter protein, an ion channel, a membrane protein, a secreted protein, a transcription factor, an enzyme, a peptide or protein hormone, a growth factor, a structural protein, a cytoplasmic protein, a cytoskeletal protein, a viral antigen, a bacterial antigen, a protozoan antigen, an allergen, a tumor antigen, or fragments, variants, or combinations of any of these.
Item 36A: The pre-filled syringe of item 34, wherein the at least one peptide or protein is selected or derived from an antigen or epitope of a pathogen, preferably selected or derived from a Coronavirus, or a fragment or variant of any of these.
Item 36B: The pre-filled syringe any one of the preceding items, wherein the RNA comprises a coding sequence encoding a pre-fusion stabilized spike protein comprising at least one pre-fusion stabilizing K986P and V987P mutation, preferably wherein the at least one antigenic peptide or protein comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 1 or 2 or an immunogenic fragment or immunogenic variant of any of these.
Item 36C: The pre-filled syringe any one of the preceding items, wherein the RNA comprises a coding sequence encoding a pre-fusion stabilized spike protein comprising at least one pre-fusion stabilizing K986P and V987P mutation, preferably wherein the coding sequence comprises or consists of a G/C optimized coding sequence comprising a nucleic acid sequence being identical to SEQ ID NOs: 3 or 4 or a fragment or variant thereof.
Item 36D: The pre-filled syringe any one of the preceding items, wherein the RNA comprises a coding sequence encoding a pre-fusion stabilized spike protein comprising at least one pre-fusion stabilizing K986P and V987P mutation, preferably wherein the RNA comprises or consists of a nucleic acid sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 5 to 10 or a fragment or variant of that sequence.
Item 37: The pre-filled syringe of any one of the preceding items, wherein the RNA is formulated in at least one cationic or polycationic compound, e.g. cationic or polycationic peptides, proteins, lipids, polysaccharides, and/or polymers.
Item 38: The pre-filled syringe of any one of the preceding items, wherein the RNA is formulated in lipid-based carriers, preferably wherein the lipid-based carriers encapsulate the RNA.
Item 39: The pre-filled syringe of any one of the items 2a or 38, wherein the concentration of lipid of the pharmaceutical composition contained in the pre-filled syringe is in a range from about 2.5 μg/ml to about 12.50 mg/ml.
Item 40: The pre-filled syringe of any one of the items 2a to 39, wherein the wt/wt ratio of lipid to the RNA in the lipid-based carriers is from about 10:1 to about 60:1, preferably from about 20:1 to about 30:1, more preferably about 25:1.
Item 41: The pre-filled syringe of any one of the items 2a to 40, wherein the N/P ratio of the lipid-based carriers to the RNA is in a range from about 1 to about 10, preferably in a range from about 5 to about 7, more preferably about 6.
Item 42: The pre-filled syringe of any one of the items 2a to 41, wherein the lipid-based carriers have a polydispersity index (PDI) value of less than about 0.3, preferably of less than about 0.2, more preferably of less than about 0.1.
Item 43: The pre-filled syringe of any one of the items 2a to 42, wherein the lipid-based carriers have a Z-average size in a range from about 50 nm to about 150 nm, preferably in a range from about 50 nm to about 120 nm, more preferably in a range of about 60 nm to about 115 nm.
Item 44: The pre-filled syringe of any one of the items 2a to 43, wherein the lipid-based carriers are liposomes, lipid nanoparticles, lipoplexes, and/or nanoliposomes.
Item 45: The pre-filled syringe of any one of the items 2a to 44, wherein the lipid-based carriers are lipid nanoparticles.
Item 46: The pre-filled syringe of any one of the items 2a to 45, wherein the lipid-based carriers are lipid nanoparticles that encapsulate the RNA.
Item 47: The pre-filled syringe of any one of the items 2a to 46, wherein the lipid-based carriers comprise at least one aggregation-reducing lipid, at least one cationic lipid, at least one neutral lipid, and/or at least one steroid or steroid analogue.
Item 48: The pre-filled syringe of item 47, wherein the aggregation reducing lipid is a polymer conjugated lipid, e.g. a PEG-conjugated lipid.
Item 49a: The pre-filled syringe of item 48, wherein the polymer conjugated lipid is a PEG-conjugated lipid according to formula (IVa) or derived from formula (IVa), wherein n has a mean value ranging from 30 to 60, preferably wherein n has a mean value of about 45, 46, 47, 48, 49, 50, 51, 52, 53, 54, more preferably wherein n has a mean value of 45 or 49.
Item 49b: The pre-filled syringe of item 48 or 49a, wherein the polymer conjugated lipid is a PEG-conjugated lipid selected or derived from DMG-PEG 2000, C10-PEG2K, Cer8-PEG2K, or ALC-0159 (lipid of formula IVa), preferably ALC-0159.
Item 50a: The pre-filled syringe of any one of item 47 to 49, wherein the at least one cationic lipid is a lipid according to formula (III) or a lipid derived from formula (III), preferably a lipid according to formula (III-3) or a lipid derived from formula (III-3).
Item 50b: The pre-filled syringe of any one of item 47 to 50a, wherein the at least one cationic lipid is a lipid selected or derived from ALC-0315 (lipid of formula III), SM-102, SS-33/4PE-15, HEXA-C5DE-PipSS, or compound C26, preferably ALC-0315.
Item 51: The pre-filled syringe of any one of item 47 to 50, wherein the at least one neutral lipid is 1,2-distearoyl-sn-glycero-3-phosphocholine (DSPC), DHPC, or DphyPE, preferably DSPC.
Item 52: The pre-filled syringe of any one of item 47 to 51, wherein the steroid or steroid analogue is cholesterol or cholesteryl hemisuccinate (CHEMS), preferably cholesterol.
Item 53a: The pre-filled syringe of any one of item 2a to 52, wherein the lipid-based carriers of the pharmaceutical composition contained in the pre-filled syringe comprise
i. at least one cationic lipid, preferably as defined in item 50a or 50b;
ii. at least one neutral lipid, preferably as defined in item 51;
iii. at least one steroid or steroid analogue, preferably as defined item 52; and
iv. at least one aggregation reducing lipid, preferably as defined in items 48 to 49a/b.
Item 53b: The pre-filled syringe of any one of item 2a to 53a, wherein the lipid-based carriers of component B comprise (i) the cationic lipid ALC-0315 (lipid of formula III), (ii) the neutral lipid DSPC, (iii) cholesterol, and (iv) the aggregation reducing lipid ALC-0159 (lipid of formula IVa).
Item 53c: The pre-filled syringe of any one of item 2a to 53a, wherein the lipid-based carriers of component B comprise (i) the cationic lipid SM-102, (ii) the neutral lipid DSPC, (iii) cholesterol, and (iv) the aggregation reducing lipid DMG-PEG 2000.
Item 54: The pre-filled syringe of item 2a to 53c, wherein (i) to (iv) are in a molar ratio of about 20-60% cationic lipid, about 5-25% neutral lipid, about 25-55% steroid or steroid analogue, and about 0.5-15% aggregation reducing lipid.
Item 55: The pre-filled syringe of any one of item 2a to 53c, wherein (i) to (iv) are in a molar ratio of about 47.4% cationic lipid, 10% neutral lipid, 40.9% steroid or steroid analogue, and 1.7% aggregation reducing lipid.
Item 56: The pre-filled syringe of any one of the preceding items, wherein the pharmaceutical composition contained in the pre-filled syringe comprises less than about 500 ppM ethanol, preferably less than about 50 ppM ethanol, more preferably less than about 5 ppM ethanol.
Item 57: The pre-filled syringe of any one of the preceding items, wherein the pharmaceutical composition contained in the pre-filled syringe comprises a sugar in a concentration of about 5 mM to about 300 mM, preferably about 14 mM.
Item 58: The pre-filled syringe of any one of the preceding items, wherein the pharmaceutical composition contained in the pre-filled syringe comprises a salt in a concentration of about 10 mM to about 300 mM, preferably about 147 mM.
Item 59: The pre-filled syringe of any one of the preceding items, wherein the pharmaceutical composition contained in the pre-filled syringe is provided as a liquid composition.
Item 60: The pre-filled syringe of any one of items 1 to 59, wherein the pharmaceutical composition contained in the pre-filled syringe is provided as a frozen composition.
Item 61: The pre-filled syringe of any one of items 1 to 59, wherein the pharmaceutical composition contained in the pre-filled syringe is provided as a lyophilized or spray(freeze) dried composition.
Item 62: The pre-filled syringe of any one of the preceding items, wherein the pre-filled syringe additionally comprises a buffer for re-constitution and/or dilution of the pharmaceutical composition, preferably wherein the buffer is provided in a separate compartment of the syringe.
Item 63: The pre-filled syringe of item 62, wherein the buffer comprises a salt, preferably NaCl, optionally in a concentration of about 0.9%, and, optionally an antimicrobial preservative.
Item 64: The pre-filled syringe of any one of the preceding items, wherein the pharmaceutical composition contained in the pre-filled syringe is a vaccine.
Item 65: The pre-filled syringe of item 64, wherein the vaccine is against a pathogen, preferably against a virus, more preferably against a Coronavirus.
Item 65: The pre-filled syringe any one of the preceding items, wherein the pharmaceutical composition or vaccine contained in the pre-filled syringe comprises an RNA encoding an antigen or epitope selected or derived from a SARS-CoV-2 virus, wherein the RNA comprises or consists of a nucleic acid sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 5 to 10 or a fragment or variant of that sequence, wherein, preferably, the RNA does not comprise chemically modified nucleotides, wherein, preferably, the RNA is encapsulated in a lipid-based carriers, preferably LNPs, preferably comprising
(i) at least one cationic lipid, preferably a lipid of formula (III) of WO2018/078053A1, more preferably lipid III-3;
(ii) at least one neutral lipid, preferably 1,2-distearoyl-sn-glycero-3-phosphocholine (DSPC);
(iii) at least one steroid or steroid analogue, preferably 1,2-distearoyl-sn-glycero-3-phosphocholine (DSPC); and
(iv) at least one aggregation reducing lipid, preferably a PEG-conjugated lipid derived from formula (IVa); and
wherein (i) to (iv) are in a molar ratio of about 47.4% cationic lipid, 10% neutral lipid, 40.9% steroid or steroid analogue, and 1.7% aggregation reducing lipid, and wherein the N/P ratio of the lipid-based carriers to the RNA is in a range from about 1 to about 10, preferably in a range from about 5 to about 7, more preferably about 6.
Item 66: The pre-filled syringe of any one of the preceding items, wherein the pharmaceutical composition or vaccine contained in the syringe is stable at a temperature of about 5° C. to about 25° C. for at least 6 hours.

### First, Second, and Further Medical Uses of the Kit or the Pre-Filled Syringe:

In a third aspect, the present invention relates to the medical use of the kit or kit of parts (comprising (A) a syringe for injection and (B) a pharmaceutical composition or vaccine comprising RNA) of the first aspect, and the pre-filled syringe of the second aspect (containing a pharmaceutical composition or vaccine comprising RNA).

Notably, embodiments relating to the kit or kit of parts of the first aspect or the pre-filled syringe of the second aspect may likewise be read on and be understood as suitable embodiments of medical uses of the invention.

Accordingly, the invention provides the kit or kit of parts as defined in the first aspect for use as a medicament, and the pre-filled syringe as defined in the second aspect for use as a medicament.

In preferred embodiments, the kit or kit of parts as defined in the first aspect or the pre-filled syringe as defined in the second aspect may be used for human medical purposes and also for veterinary medical purposes, preferably for human medical purposes.

In other preferred embodiments, the kit or kit of parts as defined in the first aspect or the pre-filled syringe as defined in the second aspect may be in particular used and suitable for human medical purposes, in particular for young infants, newborns, immunocompromised recipients, pregnant and breast-feeding women, and elderly people.

In a fourth aspect, the present invention relates to second medical uses of the kit or kit of parts (comprising (A) a syringe for injection and (B) a pharmaceutical composition or vaccine comprising RNA) of the first aspect, and the pre-filled syringe of the second aspect (containing a pharmaceutical composition or vaccine comprising RNA).

In preferred aspects, the invention relates to the medical use of the kit or kit of parts of the first aspect or the pre-filled syringe of the second aspect for use in the treatment or prophylaxis of a tumour disease, or of a disorder related to such tumour disease.

Accordingly, in said embodiments, the RNA of the pharmaceutical composition may encode at least one tumour or cancer antigen and/or at least one therapeutic antibody (e.g. checkpoint inhibitor).

In other preferred aspects, the invention relates to the medical use of the kit or kit of parts of the first aspect or the pre-filled syringe of the second aspect use in the treatment or prophylaxis of a genetic disorder or condition.

Such a genetic disorder or condition may be a monogenetic disease, i.e. (hereditary) disease, or a genetic disease in general, diseases which have a genetic inherited background and which are typically caused by a defined gene defect and are inherited according to Mendel's laws.

Accordingly, in said embodiments, the RNA of the pharmaceutical composition may encode a CRISPR-associated endonuclease or another protein or enzyme suitable for genetic engineering. Such a composition may also comprise a guide RNA.

In other preferred aspects, the invention relates to the medical use of the kit or kit of parts of the first aspect or the pre-filled syringe of the second aspect in the treatment or prophylaxis of a protein or enzyme deficiency or protein replacement.

Accordingly, in said embodiments, the RNA of the pharmaceutical composition may encode at least one protein or enzyme. “Protein or enzyme deficiency” in that context has to be understood as a disease or deficiency where at least one protein is deficient, e.g. A1AT deficiency.

In yet another aspect, the invention relates to the medical use of the provided kit or kit of parts of the first aspect or the pre-filled syringe of the second aspect in the treatment or prophylaxis of autoimmune diseases, allergies or allergic diseases, cardiovascular diseases, neuronal diseases, diseases of the respiratory system, diseases of the digestive system, diseases of the skin, musculoskeletal disorders, disorders of the connective tissue, neoplasms, immune deficiencies, endocrine, nutritional and metabolic diseases, eye diseases, and ear diseases.

In particularly preferred aspects, the invention relates to the medical use of the provided kit or kit of parts of the first aspect or the pre-filled syringe of the second aspect in the treatment or prophylaxis of an infection, or of a disorder related to such an infection.

In that context, an infection may be caused by a pathogen selected from a bacterium, a protozoan, or a virus, e.g. from a pathogen provided in List 1. In preferred embodiments, the pathogen is a virus, e.g. a Coronavirus (e.g. SARS-CoV-2).

Accordingly, the invention relates to the medical use of the provided kit or kit of parts of the first aspect or the pre-filled syringe of the second aspect in the treatment or prophylaxis of an infection with a Coronavirus, preferably a SARS-CoV-2 coronavirus, or of a disorder related to such an infection.

In the context of a use in the treatment or prophylaxis of an infection, the kit or kit of parts of the first aspect or the pre-filled syringe of the second aspect may preferably be administered locally or systemically. In that context, administration may be by an intradermal, subcutaneous, intranasal, or intramuscular route. In embodiments, administration may be by conventional needle injection. Preferred is intramuscular injection. Alternatively, administration may be intravenous. Alternatively, administration may be intraocular (e.g. intravitreal).

In embodiments, the administration to a subject is performed more than once, preferably more than once a day, more than once a week, or more than once a month.

In embodiments, the RNA as comprised in the pharmaceutical composition as defined in the first or second aspect is provided in an amount of about 100 ng to about 500 ug, in an amount of about 1 ug to about 200 ug, in an amount of about 1 ug to about 100 ug, in an amount of about 5 ug to about 100 ug, preferably in an amount of about 10 ug to about 50 ug, specifically, in an amount of about 1 ug, 2 ug, 3 ug, 4 ug, 5 ug, 10 ug, 15 ug, 20 ug, 25 ug, 30 ug, 35 ug, 40 ug, 45 ug, 50 ug, 55 ug, 60 ug, 65 ug, 70 ug, 75 ug, 80 ug, 85 ug, 90 ug, 95 ug or 100 ug. Notably, the amount relates to the total amount of RNA comprised in the composition or vaccine.

In the context of a use in the treatment or prophylaxis of an infection, the immunization protocol for the treatment or prophylaxis of a subject against at least one pathogen, e.g. against a Coronavirus, preferably SARS-CoV-2 comprises one single dose. For example, one single dose of about 1 μg to about 12 μg. Alternatively, the immunization protocol for the treatment or prophylaxis of a subject against at least one pathogen, e.g. against a Coronavirus, preferably SARS-CoV-2 comprises two dose. For example, two dose of about 1 μg to about 12 μg each dose.

In preferred embodiments, the vaccination/immunization immunizes the subject against a Coronavirus infection (upon administration as defined herein) for at least 1 year, preferably at least 2 years. In preferred embodiments, the vaccine/composition immunizes the subject against a Coronavirus infection for more than 2 years, more preferably for more than 3 years, even more preferably for more than 4 years, even more preferably for more than 5-10 years.

### Method of Treatment of the Kit or the Pre-Filled Syringe:

In a fifth aspect, the present invention relates to a method of treating or preventing a disorder or condition.

Notably, embodiments relating to the kit or kit of parts of the first aspect, the pre-filled syringe of the second aspect, or the medical uses of the third and fourth aspects may likewise be read on and be understood as suitable embodiments of methods of treatment as provided herein.

Furthermore, specific features and embodiments relating to method of treatments as provided herein may also apply for medical uses of the invention.

Preventing (inhibiting) or treating a disease, in particular a virus infection relates to inhibiting the full development of a disease or condition, for example, in a subject who is at risk for a disease such as a virus infection. “Treatment” refers to a therapeutic intervention that ameliorates a sign or symptom of a disease or pathological condition after it has begun to develop. The term “ameliorating”, with reference to a disease or pathological condition, refers to any observable beneficial effect of the treatment. Inhibiting a disease can include preventing or reducing the risk of the disease, such as preventing or reducing the risk of viral infection. The beneficial effect can be evidenced, for example, by a delayed onset of clinical symptoms of the disease in a susceptible subject, a reduction in severity of some or all clinical symptoms of the disease, a slower progression of the disease, a reduction in the viral load, an improvement in the overall health or well-being of the subject, or by other parameters that are specific to the particular disease. A “prophylactic” treatment is a treatment administered to a subject who does not exhibit signs of a disease or exhibits only early signs for the purpose of decreasing the risk of developing pathology.

In preferred embodiments, the disorder is an infection with a pathogen selected from a bacterium, a protozoan, or a virus, for example from a pathogen provided in List 1. In preferred embodiments, the pathogen is a virus, e.g. a Coronavirus (e.g. SARS-CoV-2).

In particularly preferred embodiments, the disorder an infection with a Coronavirus, or a disorder related to such infections, in particular an infection with SARS-CoV-2, or a disorder related to such infections (e.g. COVID-19).

In other embodiments, the disorder is a tumour disease or a disorder related to such tumour disease, a protein or enzyme deficiency, or a genetic disorder or condition.

In preferred embodiments, the present invention relates to a method of treating or preventing a disorder, wherein the method comprises applying or administering to a subject in need thereof the kit or kit of parts of the first aspect or the pre-filled syringe of the second aspect.

In particularly preferred embodiments, the subject in need is a mammalian subject, preferably a human subject, e.g. new-born human subject, pregnant human subject, immunocompromised human subject, and/or elderly human subject.

In particular, the method treating or preventing a disorder may comprise the steps of:
(A) obtaining a pre-filled syringe as defined in the context of the second aspect; or
(B) obtaining a kit or kit of parts as defined in the context of the first aspect and (B1) preparing a syringe comprising RNA, preferably RNA formulated in lipid-based carriers; and
(C) injecting the pharmaceutical composition comprising RNA formulated in lipid-based carriers into the subject in need thereof using the syringe obtained in A) or B1).

In various embodiments, the injecting step C) is comprises
C1) applying or administering said pharmaceutical composition comprising RNA formulated in lipid-based carriers to a subject as a first dose, and optionally
C2) applying or administering said composition, vaccine, or kit or kit of parts to a subject as a second dose or a further dose, preferably at least 1, 2, 3, 4, 5, 6, 7, 8, 9, 10, 11, 12 weeks 1, 2, 3, 4, 5, 6, 7, 8, 9, 10, 11, 12 months or after the first dose.

In preferred embodiments of the fourth aspect, the disorder or condition is an infection with a pathogen, preferably an infection with a Coronavirus.

### Method for Providing Stable Storage of a Pharmaceutical Composition Comprising RNA:

In a sixth aspect, the present invention relates to a method of providing a stable storage of a pharmaceutical composition or vaccine comprising RNA.

Notably, embodiments relating to the kit or kit of parts of the first aspect or the pre-filled syringe of the second aspect and the medical uses of the third and fourth aspect, or the methods of treatment of the fifth aspect may likewise be read on and be understood as suitable embodiments of the methods of providing a stable storage as provided herein.

In a preferred embodiment of the sixth aspect, the method for providing stable storage of a pharmaceutical composition or vaccine comprising RNA formulated in lipid-based carriers comprises the steps of:
a) obtaining a liquid composition or vaccine comprising RNA formulated in lipid-based carriers;
b) transferring the liquid composition or vaccine to a syringe, wherein the syringe is characterized by at least one of the following features
   (i) the inner surface of the syringe barrel and/or the syringe plunger stopper is essentially free of silicone oils;
   (ii) the syringe produces less than about 20% RNA agglomeration when incubated with an aqueous test formulation comprising RNA encapsulated in LNPs for 6 hours at 20° C.;
   (iii) the syringe produces less than about 20% RNA agglomeration when incubated with the pharmaceutical composition for 6 hours at 20° C.; or
   (v) the syringe produces less than 10 mAU*min of extractable compounds in 20 μl of a 2-Propanol extract as determined by analytical (RP)HPLC;
c) obtaining a filled syringe containing a liquid composition comprising RNA formulated in lipid-based carriers;
d) optionally, freezing of the obtained filled syringe or lyophilizing the composition contained in the obtained filled syringe;
e) stably storing the obtained filled syringe

In a particularly preferred embodiment of the sixth aspect, the method for providing stable storage of a pharmaceutical composition or vaccine comprising RNA formulated in lipid-based carriers comprises the steps of:
a) obtaining a liquid composition or vaccine comprising RNA formulated in lipid-based carriers;
b) transferring the liquid composition or vaccine to a syringe, wherein the syringe is characterized by that (i) the inner surface of the syringe barrel and/or the syringe plunger stopper is essentially free of silicone oils;
c) obtaining a filled syringe containing a liquid composition comprising RNA formulated in lipid-based carriers;
d) optionally, freezing of the obtained filled syringe or lyophilizing the composition contained in the obtained filled syringe;
e) stably storing the obtained filled syringe

In preferred embodiments of the method, the RNA is formulated in lipid-based carriers. In preferred embodiments of the method, the RNA is a single stranded. In preferred embodiments of the method, the RNA is a long chain RNA. In preferred embodiments of the method, the RNA is not an siRNA.

In preferred embodiments of the method, the syringe is characterized by that (i) the inner surface of the syringe barrel and/or the syringe plunger stopper is essentially free of silicone oils and further characterized by at least one of the features (ii), (iii), (iv) or a combination thereof.

In preferred embodiments, the composition comprising RNA is a composition as defined in the context of the first aspect (e.g. component B). In preferred embodiments, the syringe used in step b) is a syringe as defined in the context of the first aspect (e.g. component A). In particularly preferred embodiments, the obtained filled syringe is a syringe as defined in the context of the second aspect (the pre-filled syringe).

In some embodiments, the method additionally comprises a step of stably storing the obtained filled syringe for at least 30 minutes, 1 hour, 2 hours, 6 hours, 1 day, 2 days, 3 days, 4 days, 5 days, 6 days, 7 days, 2 weeks, 3 weeks, 4 weeks, 1 month, 2 months, 3 months, 4 months, 5 months, or 6 months.

In preferred embodiments, the stably storing step is performed at a storage temperature in a range from about −80° C. to about 30° C., preferably in a range from about −20° C. to about 30° C., more preferably in a range from about 5° C. to about 25° C. In specific embodiments, the storing step is performed at a storage temperature of about −80° C., about −60° C., about −40° C., about −20° C., about −10° C., abbot 0° C., about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C.

Preferably, the syringe is further characterized by any one of the features of claim the first aspect (component A), and/or wherein the pharmaceutical composition is further characterized by any one of the features of claim the first aspect (component B), and/or wherein the obtained filled syringe is further characterized by any one of the features of the second aspect.

### Method for Determining the Suitability of a Syringe for Storing Compositions Comprising RNA:

In a seventh aspect, the present invention relates to a method for determining the suitability of a syringe for storing a pharmaceutical composition comprising RNA.

As outlined herein, some syringes can produce RNA agglomeration in a composition comprising RNA, e.g. in a pharmaceutical composition of the invention. To identify syringes that are suitable for storing composition comprising RNA, novel analytical approaches have to be developed.

Notably, embodiments relating to the kit or kit of parts, the pre-filled syringe, the medical uses, the methods of treatment, or the method of providing a stable storage may likewise be read on and be understood as suitable embodiments of the methods of determining the suitability of a syringe as provided herein.

In preferred embodiments, the method for determining the suitability of a syringe for storing a composition comprising RNA comprises the following steps:
A) drawing a solvent into an empty syringe;
B) emptying the syringe to obtain a solvent extract;
C) determining the amount of compounds in the obtained solvent extract produced by the syringe;
D) assigning suitability of the syringe based on the amount of compounds determined in the solvent extract.

In preferred embodiments, the volume of solvent used in step A) and B) is in a range from 0.1 mL to about 10 mL. In particularly preferred embodiments, the volume of solvent used in step A) and B) is 1 mL.

In preferred embodiments, the solvent used in step A) and B) can be selected from any suitable organic solvent. In particular, the organic solvent can be selected from ethanol, methanol 2-propanol (isopropanol), or acetonitrile. In particularly preferred embodiments, the organic solvent is 2-propanol (isopropanol).

In preferred embodiments, step A and B are performed at least once (one draw/eject cycle), preferably at least twice (two draw/eject cycle), more preferably at least three times (three draw/eject cycles).

In particularly preferred embodiments, step A and B are performed as three draw/eject cycles.

Accordingly, to generate a solvent extract for determining compounds that may cause RNA aggregation, a certain volume of solvent (e.g. 1 mL 2-Propanol) may be drawn into an empty fresh syringe (step (A) “drawing a solvent into an empty syringe”), followed by ejection of the full volume of the solvent (step (B) “emptying the syringe to obtain a solvent extract”) into e.g. a vial. Such a step would represent one draw/eject cycle. Following such a draw/eject cycle, the ejected solvent (e.g. 1 mL 2-Propanol) may again be drawn into the same syringe, followed by another ejection of the volume into a vial. The ejected solvent (e.g. 1 mL 2-Propanol) may be again drawn into the same syringe and ejected into a vial.

The method is preferably performed at room temperature.

Optionally, an incubation step of 1 min to 24 h is between step A and B.

The obtained 2-Propanol extract (e.g. 3 times extracted; three draw/eject cycles) may be analyzed using RP-HPLC as explained above. The extraction procedure as described herein ensures that all parts of the syringe that may be in contact with the pharmaceutical composition, e.g. needle, syringe barrel, plunger stopper, are extracted or washed with the organic solvent.

In preferred embodiments, determining step (C) comprises analyzing the solvent extract. Suitably, the solvent extract is analyzed using (RP)HPLC, (RP)HPLC-CAD or mass spectrometry. In particularly preferred embodiments, the solvent extract is analyzed using (RP)HPLC.

(RP)HPLC may be performed essentially according to the following procedure:

20 μl extract may be injected into an HPLC column (e.g. an analytic C18 column). Analytical (RP)HPLC may be performed using the following conditions: Gradient 1: Buffer A: 0.1 M TEAA (pH 7.0); Buffer B: acetonitrile/methanol (50%/50%), 0.1% NH4OH. Starting at 10% buffer B, holding 10% B for 1 min, following to 100% buffer B in 15 min. Additional holding for 6 min at 100% buffer B, followed by decreasing to 10% buffer B. Flow rate of 0.5 ml/min (50° C. column temperature).

HPLC chromatograms are typically recorded at a wavelength of 260 nm. The obtained chromatograms may be evaluated using a software and the total area of detectable compounds may be determined (mAU*min). The total area of detectable compounds in the chromatogram expressed as “mAU*min” indicates the amount of detectable compounds that have been extracted from the syringe (compounds that can potentially cause RNA agglomeration). Such detectable compounds comprise extractable compounds that are soluble in organic solvents, e.g. lubricant oils comprising silicone oil. For determining the identity of extracted compounds, methods such as mass spectrometry may be used. In preferred embodiments, the analytical RP-HPLC (UV 260 nm) for analyzing the solvent extract is performed on a C18 modified analytical HPLC column, preferably a BEH C18 column.

In preferred embodiments, the method is performed according to Example 3.

In preferred embodiments, suitability of the syringe according to step D) is assigned when less than 10 mAU*min of detectable compounds are in the solvent extract, preferably 20 μl of a 2-Propanol extract, as determined by analytical RP-HPLC (UV 260 nm). In even more preferred embodiments, suitability of the syringe is assigned when less than 10 mAU*min, 9 mAU*min, 9 mAU*min, 7 mAU*min, 6 mAU*min, 5 mAU*min of detectable compounds are in the solvent extract, preferably 20 μl of a 2-Propanol extract, as determined by analytical RP-HPLC (UV 260 nm).

In preferred embodiments, the method additionally comprises a comparison to a reference standard. The reference standard may comprise a solvent extract obtained from a syringe for which suitability according to step D) has not been assigned (negative reference standard), or may comprise a solvent extract obtained from a syringe for which suitability according to step D) has been assigned (positive reference standard). Comparing the obtained solvent extract to a positive and/or a negative reference standard may streamline the analytic procedure.

In preferred embodiments, the method of the seventh aspect is for determining the suitability of a syringe for storing a composition comprising RNA, wherein the composition is a pharmaceutical composition as characterized in the context of the first aspect (“component B”). The method of the seventh aspect may be used an applied for controlling the quality or the suitability of pharmaceutical compositions comprising RNA. The method may therefore used as a quality control step in the manufacturing of the kit or kit of parts of the first aspect or the pre-filled syringe of the second aspect.

### Further Method for Determining the Suitability of a Syringe for Storing Compositions Comprising RNA:

In a eighth aspect, the present invention relates to a further method for determining the suitability of a syringe for storing a pharmaceutical composition comprising RNA.

As outlined herein, some syringes can produce RNA agglomeration in a composition comprising RNA, e.g. in a pharmaceutical composition of the invention. To identify syringes that are suitable for storing composition comprising RNA, novel analytical approaches have to be developed.

Notably, embodiments relating to the kit or kit of parts, the pre-filled syringe, the medical uses, the methods of treatment, or the method of providing a stable storage may likewise be read on and be understood as suitable embodiments of the methods of determining the suitability of a syringe as provided herein.

In preferred embodiments, the method for determining the suitability of a syringe for storing a composition comprising RNA comprises the following steps:
A) drawing a composition comprising RNA into an empty syringe;
B) incubating the syringe containing said composition;
C) determining the amount of RNA agglomeration in the composition produced by the syringe after incubation;
D) assigning suitability of the syringe based on the amount of RNA agglomeration.

In preferred embodiments, the volume of composition used in step A) and B) is in a range from 0.1 mL to about 10 mL, preferably 1 mL.

In preferred embodiments, the incubation step B) is performed for about 30 min to about 1d, preferably for about 6 h.

In preferred embodiments, the incubation step B) is performed at a temperature ranging from −80° C. to about 20° C., preferably ranging from about −20° C. to about 20° C., more preferably ranging from about 5° C. to about 20° C.

In preferred embodiments, the composition used in the method is an aqueous test formulation comprising RNA encapsulated in LNPs (as defined in the context of the first aspect).

In other preferred embodiments, the composition used in the method is a pharmaceutical composition as characterized in the context of the first aspect

In preferred embodiments, the RNA agglomeration in step C) is determined by using analytical (RP)HPLC of RNA In various embodiments, the RNA used for (RP)HPLC analysis has been isolated from the components of the composition (e.g. the aqueous test formulation or the pharmaceutical composition).

An isolation step from the components of the composition may be required in embodiments where the composition comprises RNA that is complexed or formulated (e.g. with lipids to generate LNPs or with peptides/proteins or polymers).

Accordingly, the composition may be treated with a detergent (e.g. about 2% Triton X100) to dissociate the lipid based carrier (if used as a formulation) and to release the encapsulated RNA. In embodiments where the RNA is formulated in cationic or polycationic peptides of proteins, the RNA may be treated with e.g. heparin. The released RNA may be captured using suitable binding compounds, e.g. Agencourt AMPure XP beads (Beckman Coulter, Brea, CA, USA) essentially according to the manufacturer's instructions. Following preparation of the RNA sample, analytical (RP)HPLC may be performed to determine the amount of RNA agglomeration.

In preferred embodiments, the RNA agglomeration is determined based on the proportion of the relative peak area of the tail in the obtained chromatogram, e.g. the obtained HPLC chromatogram, preferably (RP)HPLC chromatogram.

(RP)HPLC may be performed essentially according to the following procedure:

RNA samples may be diluted to e.g. an RNA concentration of 0.05 g/I using e.g. water for injection (WFI). A certain volume (e.g. 10 μl) of the diluted RNA sample may be injected into an HPLC column (e.g. a monolithic poly(styrene-divinylbenzene), e.g. 4.6 mm×50 mm). Analytical (RP)HPLC may be performed using the following conditions: Gradient 1: Buffer A (0.1 M TEAA (pH 7.0); Buffer B (0.1 M TEAA (pH 7.0) containing 25% acetonitrile. Starting at 35% buffer B followed by an extension to 55% buffer B over 20 minutes at a flow rate of 1 ml/min (70° C. column temperature). HPLC chromatograms are typically recorded at a wavelength of 260 nm. The obtained chromatograms were evaluated using a software and the relative peak areas of the tail was determined in percent (%) as described herein. The relative area of the tail fractions in the chromatogram indicates the amount of RNA that has a longer elution time than the RNA with the expected size (which can comprise agglomerated RNA). Since the amount of the RNA injected into the HPLC is typically known, the analysis of the relative peak area of the tail fractions provides information on the amount of RNA agglomeration. That value may be used to calculate an RNA agglomeration value (RNA agglomeration [%]). To arrive at a respective RNA agglomeration value, the peak areas of the tail in % of a corresponding control sample (a control same not exposed to a syringe) may be subtracted. For example, if the relative peak area of the tail in the control sample is 10%, and the relative peak area of the tail in the sample subjected to a syringe is 30%, the calculated RNA agglomeration value is 20%. In preferred embodiments, the analytical RP-HPLC (UV 260 nm) is performed on a analytical monolithic poly(styrene-divinylbenzene column).

In preferred embodiments, the method is performed according to Example 2.

In preferred embodiments, suitability of the syringe according to step D) is assigned when less than 20% of the RNA of the composition is agglomerated upon incubation in the syringe, preferably as determined by analytical RP-HPLC (UV 260 nm).

In preferred embodiments, suitability of the syringe according to step D) is assigned when less than about 20%, 19%, 18%, 17%, 16%, 15%, 14%, 13%, 12%, 11%, 10%, 9%, 8%, 7%, 6%, 5%, 4%, 3% or 2% of the RNA of the composition is agglomerated upon incubation in the syringe, preferably as determined by analytical RP-HPLC (UV 260 nm).

In preferred embodiments, the method additionally comprises a comparison to a reference standard. The reference standard may comprise a composition obtained from a syringe for which suitability according to step D) has not been assigned (negative reference standard, e.g. comprising more than 20% RNA agglomeration), or may comprise a solvent extract obtained from a syringe for which suitability according to step D) has been assigned (positive reference standard, e.g. comprising less than 20% RNA agglomeration). Comparing the obtained composition to a positive and/or a negative reference standard may streamline the analytic procedure.

In preferred embodiments, the method of the eighths aspect is for determining the suitability of a syringe for storing a composition comprising RNA, wherein the composition is a pharmaceutical composition as characterized in the context of the first aspect (“component B”). The method of the eighths aspect may be used an applied for controlling the quality or the suitability of pharmaceutical compositions comprising RNA. The method may therefore used as a quality control step in the manufacturing of the kit or kit of parts of the first aspect or the pre-filled syringe of the second aspect.

Method for Determining RNA Agglomeration Upon Exposure with an Article

In a ninth aspect, the present invention relates to a method for determining RNA agglomeration upon exposure of a pharmaceutical composition comprising RNA with an article.

As outlined herein, some articles of manufacture (e.g. syringes) can produce RNA agglomeration in a composition comprising RNA, e.g. in a pharmaceutical composition of the invention. To identify articles of manufacture that are suitable for exposing composition comprising RNA, novel analytical approaches have to be developed.

Notably, embodiments relating to the kit or kit of parts, the pre-filled syringe, the medical uses, the methods of treatment, the method of providing a stable storage, or the methods of determining the suitability of a syringe may likewise be read on and be understood as suitable embodiments of the methods of determining RNA agglomeration upon exposure with an article as provided herein.

In preferred embodiments of the ninth aspect, the method for determining RNA agglomeration upon exposure of a composition comprising RNA with an article comprises the following steps
A) adding a composition comprising RNA into an article;
B) incubating the article comprising the composition;
C) determining the amount of RNA agglomeration in the composition after incubation produced by the article.

In preferred embodiments, the RNA agglomeration in step C) is determined by using analytical HPLC, preferably on RNA isolated from the components of the composition as described herein.

In preferred embodiments, the RNA agglomeration in % is determined based on the proportion of the relative peak area of the tail in the obtained HPLC chromatogram as described herein. Suitably, the analytical RP-HPLC (UV 260 nm) is performed on a analytical monolithic poly(styrene-divinylbenzene column) as described herein.

In preferred embodiments, the article is an article selected from a vial, a syringe, or a container, or a bag. The article may comprise at least one material selected from organic polymers (e.g. olefin polymer, cyclic olefin copolymer, polypropylene, polyester, polysterene, polyethylene, polycarbonate), glass, thermoplastic elastomers, silicone polymers, rubbers, metals, etc. In particularly preferred embodiments, the article is a syringe.

In preferred embodiments, the composition used in the method is an aqueous test formulation comprising RNA encapsulated in LNPs as defined herein (e.g. in the first aspect) or a pharmaceutical composition as defined herein (e.g. in the first aspect).

### Use of a Syringe for Storing a Pharmaceutical Composition Comprising RNA

In a tenth aspect, the present invention relates to the use of a syringe for storing a composition comprising RNA.

Notably, embodiments relating to the kit or kit of parts, the pre-filled syringe, the medical uses, the methods of treatment may likewise be read and applied to the use of a syringe as outlined herein.

In particularly preferred embodiments of the use of the tenth aspect, the syringe is characterized by at least one of the following features
(i) the inner surface of the syringe barrel and/or the syringe plunger stopper is essentially free of silicone oils;
(ii) the syringe produces less than about 20% RNA agglomeration when incubated with an aqueous test formulation comprising RNA encapsulated in LNPs for 6 hours at 20° C.;
(iii) the syringe produces less than about 20% RNA agglomeration when incubated with the pharmaceutical composition of component B for 6 hours at 20° C.; or
(iv) the syringe produces less than 10 mAU*min of extractable compounds in 20 μl of a 2-Propanol extract as determined by analytical (RP)HPLC.

In particularly preferred embodiments of the use, the syringe is characterized by that (i) the inner surface of the syringe barrel and/or the syringe plunger stopper is essentially free of silicone oils.

In preferred embodiments of the use, the RNA is formulated in lipid-based carriers. In preferred embodiments of the use, the RNA is single stranded RNA. In preferred embodiments of the use, the RNA is a long chain stranded RNA. In preferred embodiments of the use, the RNA is not an siRNA.

In preferred embodiments of the use, the syringe is characterized by that (i) the inner surface of the syringe barrel and/or the syringe plunger stopper is essentially free of silicone oils and further characterized by at least one of the features (ii), (iii), (iv) or a combination thereof.

Syringes as characterized herein are particularly suitable for the use of storing a pharmaceutical composition comprising RNA. As defined herein, “storage” may be understood as a prolonged exposure or containment of a composition in the syringe, wherein the duration of storage may be in a range of about 30 minutes to about 6 months. For example, storage may be for at least about 30 minutes, 1 hour, 2 hours, 6 hours, 1 days, 2 days, 1 week, 2 weeks, 3 weeks, 1 month, 2 months, 3 months, 4 months, 5 months, or 6 months. The temperature conditions under which storage according to the invention may occur can range from −80° C. to about 30° C., preferably in a range from about −20° C. to about 30° C., more preferably in a range from about 5° C. to about 20° C. Typically, when reference is made to “storage”, such a storage has to be understood as having a duration of about 30 minutes to about 6 months, and as having a temperature in a range from about 5° C. to about 20° C.

Methods for determining RNA agglomeration or for analyzing 2-Propanol extracts are suitably used as defined herein.

In preferred embodiments of the use, the pharmaceutical composition is selected from a pharmaceutical composition as defined in the first aspect. In preferred embodiments of the use, the syringe is selected from syringes as defined in the first aspect or as defined in the context of the second aspect.

### Use of a Syringe for Reducing or Preventing RNA Agglomeration of a Composition Comprising RNA

In a eleventh aspect, the present invention relates to the use of a syringe for reducing or preventing RNA agglomeration of a composition comprising RNA.

Notably, embodiments relating to the kit or kit of parts, the pre-filled syringe, the medical uses, the methods of treatment may likewise be read and applied to the use of a syringe as outlined herein.

In particularly preferred embodiments of the use of the eleventh aspect, the syringe is characterized by at least one of the following features
(i) the inner surface of the syringe barrel and/or the syringe plunger stopper is essentially free of silicone oils;
(ii) the syringe produces less than about 20% RNA agglomeration when incubated with an aqueous test formulation comprising RNA encapsulated in LNPs for 6 hours at 20° C.;
(iii) the syringe produces less than about 20% RNA agglomeration when incubated with the pharmaceutical composition of component B for 6 hours at 20° C.; or
(iv) the syringe produces less than 10 mAU*min of extractable compounds in 20 μl of a 2-Propanol extract as determined by analytical (RP)HPLC.

In particularly preferred embodiments of the use, the syringe is characterized by that (i) the inner surface of the syringe barrel and/or the syringe plunger stopper is essentially free of silicone oils.

In preferred embodiments of the use, the RNA is formulated in lipid-based carriers. In preferred embodiments of the use, the RNA is single stranded RNA. In preferred embodiments of the use, the RNA is a long chain stranded RNA. In preferred embodiments of the use, the RNA is not an siRNA.

In preferred embodiments of the use, the syringe is characterized by that (i) the inner surface of the syringe barrel and/or the syringe plunger stopper is essentially free of silicone oils and further characterized by at least one of the features (ii), (iii), (iv) or a combination thereof.

Methods for determining RNA agglomeration or for analyzing 2-Propanol extracts are suitably used as defined herein.

In preferred embodiments of the use, the pharmaceutical composition is selected from a pharmaceutical composition as defined in the first aspect. In preferred embodiments of the use, the syringe is selected from syringes as defined in the first aspect or as defined in the second aspect.

### First, Second, and Further Medical Uses of a Pharmaceutical Composition:

In a twelfth aspect, the present invention relates to the medical use of a pharmaceutical composition comprising RNA.

Notably, embodiments relating to the kit or kit of parts of the first aspect, the pre-filled syringe of the second aspect, the medical uses of the third and fourth aspect, and the methods of treatment of the fifth may likewise be read and applied to the medical uses as outlined herein.

As shown herein, the inventors surprisingly found that certain syringes can generate RNA agglomeration in pharmaceutical compositions comprising RNA. That newly described problem of RNA agglomeration is solved by using syringes as defined herein e.g. characterized in the context of the first aspect or the second aspect (pre-filled syringe).

Accordingly, the invention provides a pharmaceutical composition comprising RNA formulated in lipid-based carriers as defined herein for use as a medicament, wherein the pharmaceutical composition is administered to a subject using a syringe for injection that is characterized by any of the features of the first aspect (in the context of component A) or using a pre-filled syringe that is characterized by any of the features of the second aspect.

Suitably, the invention provides a pharmaceutical composition comprising RNA formulated in lipid-based carriers as defined herein for use as a medicament, wherein the pharmaceutical composition is administered to a subject using a syringe for injection that is characterized by that (i) the inner surface of the syringe barrel and/or the syringe plunger stopper is essentially free of silicone oils.

In various embodiments, the pharmaceutical composition comprising RNA formulated in lipid-based carriers has undergone storage in the syringe before the administration to the subject. Suitably, the pharmaceutical composition is stable upon the storage. Stability and storage conditions are preferably as defined in the context of the first or the second aspect.

Suitably, the less than 20% of the RNA of the contained pharmaceutical composition is agglomerated when administered to the subject.

Notably, the pharmaceutical composition comprising RNA formulated in lipid-based carriers is suitably characterized by features of the first and second aspect.

In preferred embodiments in that context, the RNA is single stranded RNA. In preferred embodiments in that context, the RNA is a long chain RNA. In preferred embodiments in that context, the RNA is not an siRNA. In preferred embodiments in that context, the pharmaceutical composition does not comprise a peptide or a protein based medicament.

In preferred embodiments, the pharmaceutical composition is a vaccine as defined herein.

In preferred embodiments in that context, the syringe used for administration is further characterized at least one of the following features
(ii) the syringe produces less than about 20% RNA agglomeration when incubated with an aqueous test formulation comprising RNA encapsulated in LNPs for 6 hours at 20° C.;
(iii) the syringe produces less than about 20% RNA agglomeration when incubated with the pharmaceutical composition of component B for 6 hours at 20° C.;
(iv) the syringe produces less than 10 mAU*min of extractable compounds in 20 μl of a 2-Propanol extract as determined by analytical (RP)HPLC; or
(v) a combination of any of (ii) to (iv).

In preferred embodiments, the pharmaceutical composition or vaccine comprising RNA formulated in lipid-based carriers as defined herein may be used for human medical purposes and also for veterinary medical purposes, preferably for human medical purposes.

In other preferred embodiments, the pharmaceutical composition or vaccine comprising RNA formulated in lipid-based carriers as defined herein may be in particular used and suitable for human medical purposes, in particular for young infants, new-borns, immunocompromised recipients, pregnant and breast-feeding women, and elderly people.

Notably, the syringe used for administration of the pharmaceutical composition or vaccine as defined herein is further characterized by any of the features provided in the context of the first aspect (component A) or the second aspect (pre-filled syringe).

In another aspect, the invention provides a pharmaceutical composition comprising RNA formulated in lipid-based carriers as defined herein for use in the treatment or prophylaxis of tumour disease, wherein the pharmaceutical composition is administered to a subject using a syringe for injection that is characterized by any of the features of the first aspect (in the context of component A) or using a pre-filled syringe that is characterized by any of the features of the second aspect. Preferably, the syringe for injection that is characterized by that (I) the inner surface of the syringe barrel and/or the syringe plunger stopper is essentially free of silicone oils.

Accordingly, in said embodiments, the RNA of the pharmaceutical composition may encode at least one tumour or cancer antigen and/or at least one therapeutic antibody (e.g. checkpoint inhibitor).

In other aspects, the invention provides a pharmaceutical composition comprising RNA formulated in lipid-based carriers as defined herein for use in the treatment or prophylaxis of a genetic disorder or condition, wherein the pharmaceutical composition is administered to a subject using a syringe for injection that is characterized by any of the features of the first aspect (in the context of component A) or using a pre-filled syringe that is characterized by any of the features of the second aspect. Preferably, the syringe for injection that is characterized by that (i) the inner surface of the syringe barrel and/or the syringe plunger stopper is essentially free of silicone oils.

Such a genetic disorder or condition may be a monogenetic disease, i.e. (hereditary) disease, or a genetic disease in general, diseases which have a genetic inherited background and which are typically caused by a defined gene defect and are inherited according to Mendel's laws.

Accordingly, in said embodiments, the RNA of the pharmaceutical composition may encode a CRISPR-associated endonuclease or another protein or enzyme suitable for genetic engineering. Such a composition may also comprise a guide RNA.

In other aspects, the invention provides a pharmaceutical composition comprising RNA formulated in lipid-based carriers as defined herein for use in the treatment or prophylaxis of a protein or enzyme deficiency or protein replacement, wherein the pharmaceutical composition is administered to a subject using a syringe for injection that is characterized by any of the features of the first aspect (in the context of component A) or using a pre-filled syringe that is characterized by any of the features of the second aspect. Preferably, the syringe for injection that is characterized by that (I) the inner surface of the syringe barrel and/or the syringe plunger stopper is essentially free of silicone oils.

Accordingly, in said embodiments, the RNA of the pharmaceutical composition may encode at least one protein or enzyme. “Protein or enzyme deficiency” in that context has to be understood as a disease or deficiency where at least one protein is deficient, e.g. A1AT deficiency.

In yet another aspects, the invention provides a pharmaceutical composition comprising RNA formulated in lipid-based carriers as defined herein for use in the treatment or prophylaxis of autoimmune diseases, allergies or allergic diseases, cardiovascular diseases, neuronal diseases, diseases of the respiratory system, diseases of the digestive system, diseases of the skin, musculoskeletal disorders, disorders of the connective tissue, neoplasms, immune deficiencies, endocrine, nutritional and metabolic diseases, eye diseases, and ear diseases, wherein the pharmaceutical composition is administered to a subject using a syringe for injection that is characterized by any of the features of the first aspect (in the context of component A) or using a pre-filled syringe that is characterized by any of the features of the second aspect.

Preferably, the syringe for injection that is characterized by that (i) the inner surface of the syringe barrel and/or the syringe plunger stopper is essentially free of silicone oils.

In preferred aspects, the invention provides a pharmaceutical composition comprising RNA formulated in lipid-based carriers as defined herein for use in the treatment or prophylaxis of an infection, or of a disorder related to such an infection, wherein the pharmaceutical composition is administered to a subject using a syringe for injection that is characterized by any of the features of the first aspect (in the context of component A) or using a pre-filled syringe that is characterized by any of the features of the second aspect. Preferably, the syringe for injection that is characterized by that (i) the inner surface of the syringe barrel and/or the syringe plunger stopper is essentially free of silicone oils.

In that context, an infection may be caused by a pathogen selected from a bacterium, a protozoan, or a virus, e.g. from a pathogen provided in List 1. In preferred embodiments, the pathogen is a virus, e.g. a Coronavirus (e.g. SARS-CoV-2).

Accordingly, the invention relates to the medical use of the pharmaceutical composition comprising RNA formulated in lipid-based carriers in the treatment or prophylaxis of an infection with a Coronavirus, preferably a SARS-CoV-2 coronavirus, or of a disorder related to such an infection, wherein the pharmaceutical composition is administered to a subject using a syringe for injection that is characterized by any of the features of the first aspect (in the context of component A) or using a pre-filled syringe that is characterized by any of the features of the second aspect. Preferably, the syringe for injection that is characterized by that (i) the inner surface of the syringe barrel and/or the syringe plunger stopper is essentially free of silicone oils.

In embodiments, the RNA as comprised in the pharmaceutical composition as defined herein is provided in an amount of about 100 ng to about 500 ug, in an amount of about 1 ug to about 200 ug, in an amount of about 1 ug to about 100 ug, in an amount of about 5 ug to about 100 ug, preferably in an amount of about 10 ug to about 50 ug, specifically, in an amount of about 1 ug, 2 ug, 3 ug, 4 ug, 5 ug, 10 ug, 15 ug, 20 ug, 25 ug, 30 ug, 35 ug, 40 ug, 45 ug, 50 ug, 55 ug, 60 ug, 65 ug, 70 ug, 75 ug, 80 ug, 85 ug, 90 ug, 95 ug or 100 ug. Notably, the amount relates to the total amount of RNA comprised in the composition or vaccine.

In embodiments, the administration to a subject is performed more than once, preferably more than once a day, more than once a week, or more than once a month.

In embodiments, the administration to a subject is performed intramuscularly or intraocularly.

### Method of Treatment of a Pharmaceutical Composition:

In a thirteenth aspect, the present invention relates to a method of treating or preventing a disorder or condition.

Notably, embodiments relating to the kit or kit of parts of the first aspect, the pre-filled syringe of the second aspect, the medical uses of the third and fourth aspect, and the methods of treatment of the fifth aspect may likewise be read and applied to the medical uses as outlined herein.

In particular, embodiments described in the context of the twelfth aspect may likewise apply the embodiments of the thirteenth aspect.

In particular, the invention relates to a method of treating or preventing a disease, disorder or condition, wherein the method comprises applying or administering to a subject in need thereof an effective amount of a pharmaceutical composition or vaccine comprising RNA formulated in lipid-based carriers, wherein the applying or administering to a subject is performed by using a syringe for injection that is characterized by any of the features of the first aspect (in the context of component A) or using a pre-filled syringe that is characterized by any of the features of the second aspect. Preferably, the syringe for injection that is characterized by that (i) the inner surface of the syringe barrel and/or the syringe plunger stopper is essentially free of silicone oils.

Notably, the pharmaceutical composition comprising RNA formulated in lipid-based carriers is suitably characterized by features of the first and second aspect.

In preferred embodiments in that context, the RNA is single stranded RNA. In preferred embodiments in that context, the RNA is a long chain RNA. In preferred embodiments in that context, the RNA is not an siRNA. In preferred embodiments in that context, the pharmaceutical composition does not comprise a peptide or a protein based medicament.

In preferred embodiments, the pharmaceutical composition is a vaccine as defined herein.

In preferred embodiments in that context, the syringe used for administration is further characterized at least one of the following features
(ii) the syringe produces less than about 20% RNA agglomeration when incubated with an aqueous test formulation comprising RNA encapsulated in LNPs for 6 hours at 20° C.;
(iii) the syringe produces less than about 20% RNA agglomeration when incubated with the pharmaceutical composition of component B for 6 hours at 20° C.;
(iv) the syringe produces less than 10 mAU*min of extractable compounds in 20 μl of a 2-Propanol extract as determined by analytical (RP)HPLC; or
(v) a combination of any of (ii) to (iv).

Notably, the syringe used for administration of the pharmaceutical composition or vaccine as defined herein is further characterized by any of the features provided in the context of the first aspect (component A) or the second aspect (pre-filled syringe).

In preferred embodiments, the disorder is an infection with a pathogen selected from a bacterium, a protozoan, or a virus, for example from a pathogen provided in List 1. In preferred embodiments, the pathogen is a virus, e.g. a Coronavirus (e.g. SARS-CoV-2).

In particularly preferred embodiments, the disorder an infection with a Coronavirus, or a disorder related to such infections, in particular an infection with SARS-CoV-2, or a disorder related to such infections (e.g. COVID-19).

In other embodiments, the disorder is a tumour disease or a disorder related to such tumour disease, a protein or enzyme deficiency, or a genetic disorder or condition.

In particularly preferred embodiments, the subject in need is a mammalian subject, preferably a human subject, e.g. new-born human subject, pregnant human subject, immunocompromised human subject, and/or elderly human subject.

### BRIEF DESCRIPTION OF LISTS AND TABLES

Table A: Preferred syringe barrels of SOF-S1, SOF-S2 and SOF-S3
Table B: Preferred syringe plunger stopper of SOF-S1, SOF-S2 and SOF-S3
List 1: Suitable pathogens of the invention
Item list: Pre-filled syringe containing a pharmaceutical composition comprising RNA
Table 1: mRNA used in the examples
Table 2: Lipid-based carrier composition of the examples
List 2: Syringes used in the Examples section (Examples 2 to 4)
Table 3: Effect of syringes S1 and S5 on the test formulation comprising RNA encapsulated in LNP
Table 4: Effect of syringes S5 and S6 on the test formulation comprising RNA encapsulated in LNP
Table 5: Effect of syringes S9 and S11 on the test formulation comprising RNA encapsulated in LNP
Table 6: Determined compounds (UV 260 nm) obtained by 2-Propanol extraction
Table 7: Washed syringes show reduced RNA agglomeration on the TF comprising RNA encapsulated in LNP
Table 8: Effect of syringes SOF-S1, SOF-S2, and SOF-S3 on the TF comprising RNA encapsulated in LNP

### BRIEF DESCRIPTION OF THE DRAWINGS

FIG. 1 shows a section of an exemplary chromatograms (overlay) obtained in the analysis of RNA agglomeration produced by a syringe (see e.g. Example 2). FIG. 1A shows the test formulation (TF) comprising RNA formulated in LNPs that has not been exposed to a syringe. FIG. 1B shows the TF that has been exposed to certain syringes. As indicated by the arrows in 1B, additional peaks in the tail of the chromatogram are occurring in the TF that has been exposed to certain syringes. (1) indicates the front peaks, (2) and the grey area indicates the main peak (comprising the desired RNA product), (3) indicates the tail peaks (that may comprise RNA agglomerates). Arrows indicates RNA agglomeration peaks produced by certain syringes.

FIG. 2 shows a section of an exemplary chromatogram (overlay; only main and tail) obtained in the analysis of RNA agglomeration produced by a syringe (see e.g. Example 2). As indicated by the arrows, additional peaks in the tail of the chromatogram are occurring in the TF that has been exposed to syringes S1 or S5. (2) indicates the main peak (comprising the desired RNA product), (3) indicates the tail peaks (that may comprise RNA agglomerates). Arrows indicates prominent RNA agglomeration peaks produced by a syringes S1 or S5.

### EXAMPLES

In the following, examples illustrating various embodiments and aspects of the invention are presented. However, the present invention shall not to be limited in scope by the specific embodiments presented herein, and should rather be understood as being applicable to other compositions and/or vaccines and/or uses as for example defined in the specification. Accordingly, the following preparations and examples are given to enable those skilled in the art to more clearly understand and to practice the present invention. Indeed, various modifications of the invention in addition to those described herein will become readily apparent to those skilled in the art from the foregoing description, accompanying figures and the examples below.

### Example 1: Preparation of Compositions Comprising Lipid-Based Carriers Encapsulating an RNA

The present example provides methods of obtaining the RNA of the invention as well as methods of generating a composition or a vaccine of the invention comprising RNA, in particular RNA formulated in lipid-based carriers.

### 1.1. Preparation of DNA Templates for RNA In Vitro Transcription:

DNA sequences encoding a Coronavirus spike antigen (full length prefusion stabilized SARS-CoV-2 spike protein comprising K986P, V987P substitutions) were prepared and used for subsequent RNA in vitro transcription reactions. Said DNA sequences were prepared by modifying the wild type encoding DNA sequences by introducing a G/C optimized coding sequence for stabilization and expression optimization. Sequences were introduced into a pUC derived DNA vector to comprise a stabilizing 3′-UTR sequences and a stretch of adenosines (A64), a histone-stem-loop (hSL) structure and a stretch of 30 cytosines (C30) (see Table 1). The obtained plasmid DNA templates were transformed and propagated in bacteria using common protocols known in the art. Eventually, the plasmid DNA templates were extracted, purified, and used for linearization reaction using EcoRI as digestion enzyme.

TABLE 1
mRNA construct used in Examples

| | | SEQ ID | SEQ ID | SEQ ID |
|---|---|---|---|---|
| RNA ID | Name | NO: Protein | NO: CDS | NO: mRNA |
| R9515 | SARS-CoV-2 | SEQ ID | SEQ ID | SEQ ID |
| | Spike protein | NO: 1 | NO: 3 | NO: 5 |

1.3. RNA In Vitro Transcription from Plasmid DNA Templates:

A linearized DNA template encoding R9515 was used for DNA dependent RNA in vitro transcription using T7 RNA polymerase in the presence of a sequence optimized nucleotide mixture (ATP/GTP/CTP/UTP) and cap analog (for Cap1: m7G(5′)ppp(5′)(2′OMeA)pG) under suitable buffer conditions. After RNA in vitro transcription, the obtained RNA IVT reaction comprising the mRNA was subjected to purification steps comprising TFF and RP-HPLC.

1.4. Preparation of Lipid-Based Carriers (LNP) Encapsulating the mRNA:

An ethanolic lipid solution was prepared by solubilizing the cationic lipid according to formula III-3, DSPC, cholesterol, and the aggregation reducing lipid (PEG-conjugated lipid) according to formula IVa in ethanol at a molar ratio of approximately 47.4:10:40.9:1.7. (see Table 2).

TABLE 2
Lipid-based carrier composition of the examples

| | | Ratio | | |
|---|---|---|---|---|
| | Compounds | (mol %) | Structure | Mass |
| 1 | Cholesterol | 40.9 | 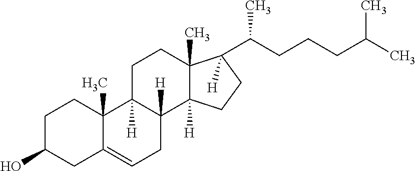 | 386.4 |
| 2 | 1,2-distearoyl- sn-glycero-3- phosphocholine (DSPC) | 10 | 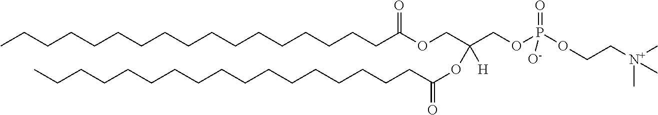 [SMILES: CCCCCCCCCCCCCCCCCC(=O)OCC(COP(=O)([O-])OCC[N+](C)(C)C)OC(=O)CCCCCCCCCCCCCCCCC] | 789.6 |
| 3 | Cationic Lipid (ALC-0315) | 47.4 | 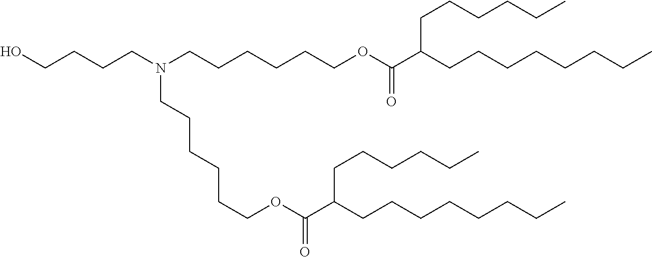 [SMILES: CCCCCCCCC(CCCCCC)C(=O)OCCCCCCN(CCCCO)CCCCCCOC(=O)C(CCCCCC)CCCCCCCC] | 765.7 |
| 4 | PEG Lipid (ALC-0159) | 1.7 | 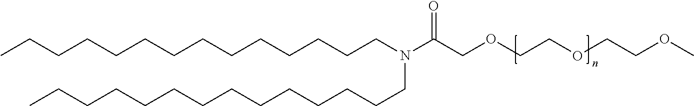 | 2010.1 |
| | | | Average n = ~49 | |

An aqueous RNA solution was prepared by adjusting the purified RNA (obtained according to Example 1.3) to a concentration of about 0.2 mg/mL in 50 mM citrate buffer, pH 4.0.

Lipid nanoparticles were prepared according to the general procedures described in PCT Pub. Nos. WO 2015/199952, WO 2017/004143 and WO 2017/075531, the full disclosures of which are incorporated herein by reference.

In short, lipid nanoparticles (LNP) were prepared at a ratio of mRNA to total Lipid of 0.03-0.04 w/w. Pumps were used to combine the ethanolic lipid solution with a flow rate F1 and the mRNA aqueous solution with a flow rate F2 at a ratio of about 1:5 to 1:3 (vol/vol) in a T-piece system. F1 and/or F2 were adjusted to flow rates above 15 ml/min to allow the formation of LNPs encapsulating the RNA that have a Z-average size in a range from about 60 nm to about 115 nm. After formulation, the ethanol was removed by at least one TEE step and at least one clarifying filtration step. After clarifying filtration, the filtrate was adjusted to a desired concentration (typically 1 g/l RNA) using a buffer comprising 150 mM sucrose, 75 mM sodium chloride, 10 mM sodium phosphate, pH 7.4. Subsequently, the resulting formulation was filtered through sterilizing filters to reduce bioburden.

### Example 2: Screening of Syringes Suitable for Compositions Comprising RNA

The goal of the experiment was to test the stability of RNA formulations obtained according to Example 1 in respective syringes for injection. Unexpectedly it has been found that some syringes produced RNA agglomeration upon storage in a syringe, an effect that has not been described in the art. RNA agglomeration is of course an unwanted effect and has to be avoided in a pharmaceutical product. Accordingly, syringe screening was performed to identify syringes that are suitable for the administration of RNA formulations.

The syringes used in the screening are provided in List 2. Certain characteristics of the syringes such as materials, coatings, and lubricants are also provided in List 2.

### List 2: Shrines Used in the Examples Section (Examples 2 to 4)

Syringe 1 (S1): Becton Dickinson, Plastipak Luer-Lok™ Tip, (polycarbonate barrel, lubricant in syringe barrel silicone oil) BD 309628.
Syringe 5 (S5): B. Braun Melsungen, Omnifix-F Luer Solo 0.01 mL-1 mL, (polypropylen barrel, Polystyrol plunger plunger stopper synthetic rubber; low amount of silicone oil) REF No. 9161406V;
Syringe 6 (S6): Becton Dickinson; Luer Slip Syringe (0.01 ml Scale); Polypropylene; <25 μg/mm² silicone oil; BD 303172;
Syringe 9 (S9): Becton Dickinson; silicone oil unknown; Flu 0.25-1 mL 23Gx1″ (0.6×25 mm) blau Ref 305832;
Syringe 11 (S11): Becton Dickinson; silicone oil unknown; Solo Shot 0.5 mL 25Gx1″ (0.5×25 mm) orange Ref 302248;
Silicone-oil free syringe S1 (SOF-S1): Terumo Plajex 1 mL Long Luer Lock SOF Ref: PJ-B1 LL2FTF1 lot: 180709B1)+PJ-R1LNBM1 Plajex 1 mL Long Plunger Lot: 190517R1; Cyclo olefin polymer (COP) barrel;
Silicone-oil free syringe S2 (SOF-S2): SiO2 medical products; 1 mL Luer+OVS Quadlayer+Crosslinked lot. 2020-728-S+1 mL Long Plunger NovaPure RU SP 4023/50G West Item 11402014; Cyclic olefin polymer (COP) barrel;
Silicone-oil free syringe S3 (SOF-S3): BD PIR3-090 Hypak SCF1 mL PRT REF: 47406710 lot: 7111128+Hypak PR1 mL PSTYP Cristal lot: 974165 Ref: 47404008+Hypak TSCF1-3 mL 4023 Flur S Lot:4237707 Ref:47190510;

### 2.1. Test Procedure

The RNA formulation comprising RNA formulated in lipid-based carriers (1 g/I RNA, drug product, DP) obtained according to Example 1 was diluted to a concentration of 10 μg/ml using 0.9% NaCl solution to obtain an aqueous test formulation (TF)

The aqueous test formulation (TF) comprises LNPs which have a molar ratio of approximately 47.4:10:40.9:1.7 proportion (mol %) of cationic lipid III-3, DSPC, cholesterol and PEG-lipid of formula (IVa) (with n=49 or with n=45). The LNPs encapsulate an RNA, wherein the RNA is the RNA according to SEQ ID NO: 5. The RNA comprises a 5′ Cap1 structure and does not comprise chemically modified nucleotides. The RNA integrity was at least about 80% (as determined using RP(HPLC)) and the encapsulation efficiency was at least 80% (as determined by a Ribogreen assay) and the Z-average particle size was in a range of about 60 nm to about 115 nm (as determined by DLS). N/P ratio of the LNPs to the RNA in the aqueous test formulation was about 6, and the wt/wt ratio of lipid to the RNA in the aqueous test formulation was about 25:1.

500 μl of diluted TF was drawn into a fresh syringe (through the needle). DP was incubated for a defined period of time (e.g. 30 min, 4 h, 6 h) at room temperature (RT) or at 5° C. in respective syringes. After incubation, the impact of incubation in the respective syringes on the mRNA was evaluated using RP HPLC as described below. Further, the impact of incubation in the respective syringes on the lipid based carrier was evaluated (e.g. encapsulation efficiency using a RiboGreen assay as known in the art, LNP particle size using DLS as known in the art, polydispersity index using DLS as known in the art).

### 2.2. Determination of the RNA Quality Upon Incubation in a Syringe

The RNA quality was determined using analytical (RP)HPLC, using a commercially available HPLC system.

Samples of the TF comprising the lipid based carrier encapsulating the RNA were treated with a detergent (3.3% (v/v) Triton X100 end concentration) to dissociate the lipid based carrier and to release the RNA. After 15 minutes incubation at room temperature, the released RNA was captured using Agencourt AMPure XP beads (Beckman Coulter, Brea, CA, USA) essentially according to the manufacturer's instructions. In short, the beads capturing the RNA were washed with 75% ethanol (3 times). Subsequently, ethanol was removed and the washed beads capturing the RNA were dried at room temperature for about 10 minutes. Next, RNA was eluted from the beads using water for injection (WFI; incubation for about 15 minutes), and the beads were removed by magnetic separation and filtration.

Following preparation of the RNA sample, analytical (RP)HPLC was performed to determine the quality of the RNA (e.g. RNA integrity in % and RNA agglomeration in %). For determining RNA integrity and RNA agglomeration, the RNA sample was diluted to a concentration of 0.05 g/I using water for injection (WFI). 10 μl of the diluted RNA sample was injected into an HPLC column (a monolithic poly(styrene-divinylbenzene column, 4.6 mm×50 mm). Analytical (RP)HPLC was performed using the following conditions: Gradient 1: Buffer A (0.1 M TEAA (pH 7.0±0.5); Buffer B (0.1 M TEAA (pH 7.0±0.5) containing 25% acetonitrile. Starting at 35% buffer B followed by an extension to 55% buffer B over 20 minutes at a flow rate of 1 ml/min (70° C. column temperature). HPLC chromatograms were recorded at a wavelength of 260 nm.

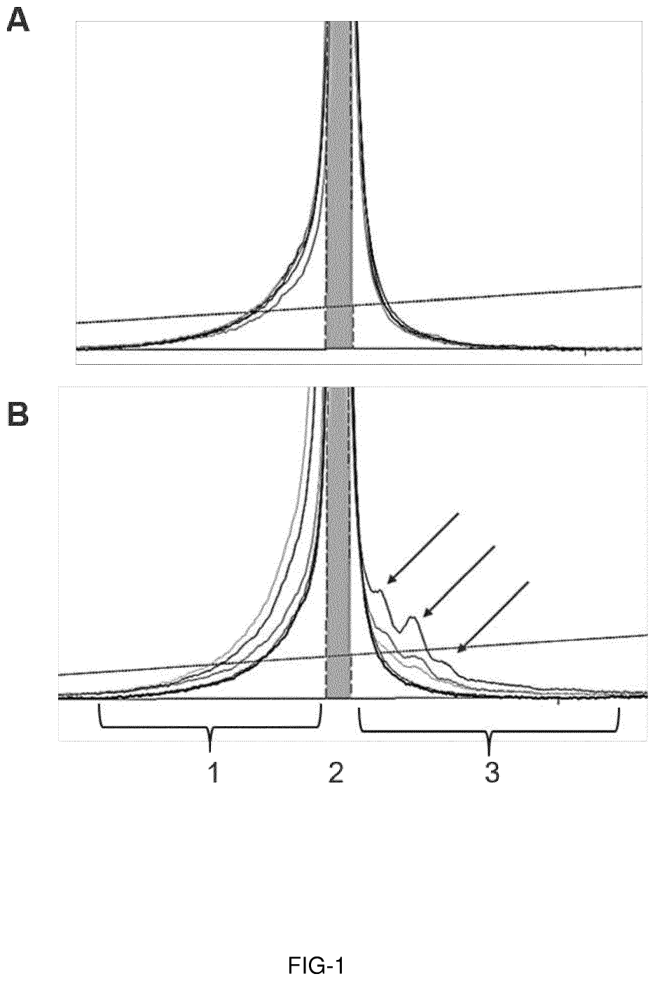

A typical HPLC chromatogram of an RNA comprises lead peaks, the main peak, and tail peaks (see e.g. FIG. 1). The main peak comprises the expected full length RNA product, the lead peaks comprise short abortive RNA fragments, and the tail peaks comprise RNA species with longer elution time, e.g. including RNA agglomerations.

The obtained HPLC chromatograms were evaluated using a software and the relative peak area of the expected RNA main peak was determined in percent (%). That value was used to assign an integrity value (RNA integrity [%]) to the sample (also referred to as “main”). The relative peak area of the main fraction indicates the amount of RNA that has 100% RNA integrity, e.g. the amount of RNA that has the expected size/length. Since the amount of the RNA injected into the HPLC is typically known, the analysis of the relative peak area of the main fraction provides information on the integrity of the RNA. For calculating the effect of the syringe on RNA integrity, RNA integrity values obtained after syringe incubation were subtracted with the RNA integrity values of a control (not incubated in a syringe). That value is indicated as “Delta RNA integrity in %” in the result Tables. For example, the value “12” means for the respective syringe, that the RNA integrity is 12% lower than the RNA integrity of the control.

In addition, the obtained chromatograms were evaluated using a software and the relative peak areas of the tail was determined in percent (%). The relative area of the tail fractions in the chromatogram indicates the amount of RNA that has a longer elution time than the RNA with the expected size (which can comprise agglomerated RNA). Since the amount of the RNA injected into the HPLC is typically known, the analysis of the relative peak area of the tail fractions provides information on the amount of RNA agglomeration. That value was used to calculate an agglomerated value (RNA agglomeration [%]). To arrive at a respective RNA agglomeration value, the peak areas of the tail in % of a corresponding control sample (the same composition not exposed to a syringe) was subtracted. For example, if the relative peak area of the tail in the control sample was 10%, and the relative peak area of the tail in the sample subjected to a syringe was 30%, the calculated RNA agglomeration value is 20%. That value is indicated as “RNA agglomeration in %” in the results Tables.

Surprisingly, some tested syringes produced RNA agglomeration, while the lipid-based carriers were not affected by a storage in the syringe (particle size, pdi, encapsulation efficiency; data not shown). As exemplarily shown in FIG. 1, RNA agglomeration does not appear in the test formulation that has not been exposed to syringes (FIG. 1A), whereas RNA agglomeration is observed after incubation of the test formulation in some syringes (FIG. 1B). As shown in FIG. 1, the peak in the chromatogram can be divided in front peaks (1), main (2) and the tail peaks (3). RNA agglomeration can be observed as the tail peak fractions increase (see also arrows in FIG. 1B).

The results are summarized in Tables 3-5.

TABLE 3
Effect of syringes S1 and S5 on the test
formulation comprising RNA encapsulated in LNP
Conditions: Storage at room temperature; 10
ng/μl RNA formulated in LNPs; Storage 6 h;

| | | | | | RNA | |
|---|---|---|---|---|---|---|
| | | | | | agglom- | Delta RNA |
| Syringe | Incubation | Front | main | tail | eration | integrity |
| type | time | peaks | peak | peaks | in % | in % |
| NO | 6 h | 37 | 54 | 9 | — | — |
| SYRINGE | | | | | | |
| S1 | 6 h | 29 | 37 | 34 | 25 | 17 |
| S1 | 6 h | 33 | 28 | 38 | 29 | 26 |
| S5 | 6 h | 32 | 52 | 16 | 7 | 2 |
| S5 | 6 h | 29 | 60 | 11 | 2 | −6 |

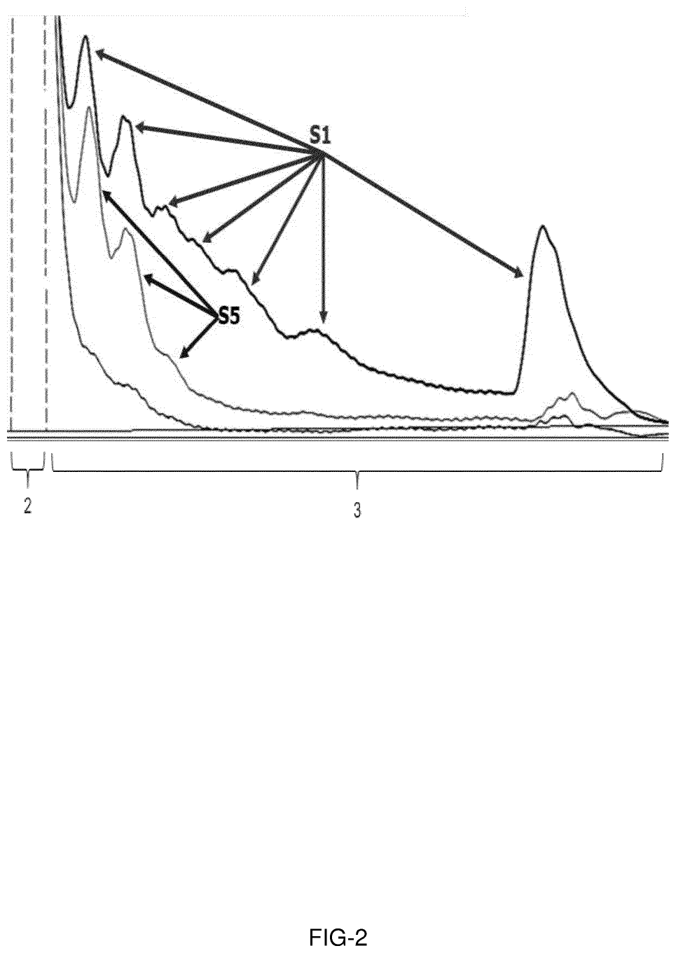

An exemplary chromatogram of that analysis is also provided in FIG. 2. As shown in FIG. 2, syringe S1 produces a stronger RNA agglomeration compared to Syringe S5.

TABLE 4
Effect of syringes S5 and S6 on the test
formulation comprising RNA encapsulated in LNP
Conditions: Storage at room temperature; 10 ng/μl
RNA formulated in LNPs; Storage 30 min or 6 h;

| | | | | | | RNA | |
|---|---|---|---|---|---|---|---|
| | | | | | | agglom- | Delta RNA |
| Syringe | Incubation | | Front | main | tail | eration | integrity |
| type | time | | peaks | peak | peaks | in % | in % |
| NO | 6 | h | 31 | 60 | 10 | — | — |
| SYRINGE | | | | | | | |
| S5 | 0.5 | h | 41 | 54 | 9 | −1 | 6 |
| S5 | 6 | h | 29 | 54 | 18 | 8 | 6 |
| S6 | 0.5 | h | 36 | 55 | 9 | −1 | 5 |
| S6 | 6 | h | 27 | 56 | 16 | 6 | 4 |

TABLE 5
Effect of syringes S9 and S11 on the test
formulation comprising RNA encapsulated in LNP
Conditions: Storage at room temperature; 10 ng/μl
RNA formulated in LNPs; Storage 30 min or 6 h;

| | | | | | | RNA | |
|---|---|---|---|---|---|---|---|
| | | | | | | agglom- | Delta RNA |
| Syringe | Incubation | | Front | main | tail | eration | integrity |
| type | time | | peaks | peak | peaks | in % | in % |
| NO | 6 | h | 26 | 64 | 10 | — | — |
| SYRINGE | | | | | | | |
| S9 | 0.5 | h | 27 | 63 | 10 | 0 | 1 |
| S9 | 6 | h | 25 | 60 | 16 | 8 | 10 |
| S11 | 0.5 | h | 28 | 63 | 9 | −1 | −1 |
| S11 | 6 | h | 28 | 63 | 9 | −1 | −1 |

### 2.3. Results of Example 2:

As surprisingly found in the present example, some syringes induced strong RNA agglomeration, an effect that has not been described in the art. Interestingly, syringe S1 that comprises silicone oil as a lubricant induced the strongest RNA agglomeration, suggesting that silicon oils (used as lubricants in syringes) are a cause of the observed RNA agglomeration.

### Example 3: Test Procedure to Identify Suitable Syringes

The present example describes a test procedure based on solvent extraction for analyzing the suitability of syringes for RNA compositions. The results show that the amount of detectable compounds in syringe extracts correlates with RNA agglomeration. Accordingly, the method as provided herein can be used to predict the suitability of syringes for RNA compositions.

### 3.1. Extraction Procedure

2-Propanol was used to extract compounds from the syringes. The 2-Propanol extract was obtained by three repeated draw/eject cycles at room temperature and with 10 syringes of the same typ. 1 mL 2-Propanol (isopropanol) was drawn into a fresh syringe (through the needle), followed by ejection of the full volume of into a vial. The ejected 1 mL 2-Propanol was again drawn from the vial into the syringe (through the needle), followed by another ejection into the vial. The ejected 2-Propanol was again drawn from the vial into the syringe ((through the needle) and ejected into a vial. The obtained 2-Propanol extract (extracted via three draw/eject cycles) was then analyzed using RP-HPLC as explained below. The extraction procedure as described herein ensured that all parts of the syringe that can be in contact with the pharmaceutical composition, e.g. needle, syringe barrel, plunger stopper, were extracted 2-Propanol.

The obtained 2-Propanol extract was analyzed using analytical RP-HPLC. 10 μl, 20 μl, 40 μl and 80 μl of the obtained 2-Propanol extract was injected into an HPLC column (Acquity UPLC Oligonucleotide BEH C18 column 130A, 1.7 μm, 2.1 mm×50 mm). Analytical (RP)HPLC was performed using the following conditions:

Buffer A: 100 mM TEAA (pH 7.0), and Buffer B: acetonitrile/methanol (50%/50%), 0.1% NH₄OH; After injection of the probe, the HPLC started with 10% buffer B, holding 10% buffer B for 1 min, following an increase to 100% buffer B in 15 min. After a holding time for 6 min at 100% buffer B, buffer B was decreased to 10%. Flow rate was 0.5 ml/min. Temperature was 50° C. HPLC chromatograms were recorded at a wavelength of 260 nm.

The result of the 2-Propanol extraction is provided in Table 6.

TABLE 6
Determined compounds (UV 260 nm)
obtained by 2-Propanol extraction

| | | Syringe 5 | | Syringe 1 |
|---|---|---|---|---|
| | | RNA | | RNA |
| Volume | Syringe 5 | Agglomeration | Syringe 1 | Agglomeration |
| [μl] | [mAU*min] | [%](6 h, RT) | [mAU*min] | [%](6 h, RT) |
| 10 | 2.83 | 2-8%* | 8.82 | 25-29%* |
| 20 | 5.68 | | 19.77 | |
| 40 | 10.12 | | 36.98 | |
| 80 | 23.16 | | 73.64 | |

*compare with Table 3 and 4

### 3.2. Re-Use of Washed Syringes and Determination of RNA Agglomeration

The Syringes that were extracted with 2-Propanol were again exposed to TF to determine whether the effect of RNA agglomeration could be reduced. RNA agglomeration was analyzed according to paragraph 2.2 and 2.3.

The result of the re-use study is provided in Table 7.

TABLE 7
Washed syringes show reduced RNA agglomeration on the
test formulation comprising RNA encapsulated in LNP

| | Incubation | | RNA agglomeration |
|---|---|---|---|
| Syringe type | time | tail peaks | in % |
| NO SYRINGE | 14 h | 13 | — |
| S1_2-Propanol washed | 14 h | 18 | 5 |
| S1_untreated | 14 h | 26 | 13 |
| S5_2-Propanol washed | 14 h | 17 | 4 |
| S5_untreated | 14 h | 20 | 7 |

### 3.3. Results of Example 3:

As shown in the example, the described 2-Propanol extraction procedure and the subsequent HPLC analysis provides an analytical method to predict the unwanted effect of syringes on RNA agglomeration. Importantly, the amount of detectable compounds in analytical HPLC correlates with the effect of RNA agglomeration (see Table 6). Furthermore, the re-use of 2-Propanol washed syringes strongly reduced the effect of RNA agglomeration (see Table 7). Accordingly, the experiments show that the amount of detectable compounds in the 2-Propanol extract (compounds may comprise silicone oils, other lubricant oils) is predictive for the effect of syringes on RNA agglomeration, and that such compounds, e.g. silicon oils are the cause of the observed RNA agglomeration.

### Example 4: Analysis of Silicone Oil Free Syringes Suitable for Pre-Filling

As suggested by the findings of example 2 and 3, silicone oil may be the cause of the observed RNA agglomeration in certain syringes. To test that, three different silicone oil free syringes suitable for obtaining pre-filled syringes were analyzed (SOF-S1, SOF-S2, SOF-S3, see List 2) according to section 2.2 and section 2.3. The results are provided in Table 8.

TABLE 8
Effect of syringes SOF-S1, SOF-S2, and SOF-S3 on
the test formulation comprising RNA encapsulated in LNP
Conditions: Storage at room temperature; TF adjusted to
0.02 g/l RNA formulated in LNPs; Storage 24 h, 48 h;

| | | | | | RNA | |
|---|---|---|---|---|---|---|
| | | | | | agglom- | Delta RNA |
| Syringe | Incubation | Front | main | tail | eration | integrity |
| type | time | peaks | peak | peaks | in % | in % |
| NO | 24 h | 34 | 57 | 10 | — | — |
| SYRINGE | | | | | | |
| NO | 48 h | 31 | 58 | 11 | — | — |
| SYRINGE | | | | | | |
| SOF-S1 | 24 h | 35 | 57 | 9 | −1 | 0 |
| SOF-S1 | 48 h | 38 | 53 | 9 | −2 | 5 |
| SOF-S2 | 24 h | 32 | 59 | 10 | 0 | −2 |
| SOF-S2 | 48 h | 34 | 56 | 9 | −2 | 1 |
| SOF-S3 | 24 h | 37 | 55 | 9 | −1 | 2 |
| SOF-S3 | 48 h | 38 | 53 | 9 | −2 | 5 |

### Results of Example 4:

As shown in Table 8, even with extended incubation period (up to 48 hours), no RNA agglomeration was detected in the three tested silicone oil free syringes, showing that the newly described problem of RNA agglomeration can be prevented in silicone-oil free pre-filled syringes.

The data clearly demonstrates that it is crucial to use silicone oil free syringes (or syringes with reduced amount of silicone oil) to prevent the formation of RNA agglomeration. This finding is particularly important for pre-filled syringes, where the pharmaceutical compositions comprising RNA are stored in the syringe for a longer period of time.

1. A kit or kit of parts comprising the following components (A) a syringe for injection, and (B) a pharmaceutical composition comprising RNA, wherein the syringe of component A is characterized by that (i) the inner surface of the syringe barrel and/or the syringe plunger stopper is essentially free of silicone oils.

2. The kit or kit of parts of claim 1, wherein the RNA of component B is formulated in lipid-based carriers.

3. The kit or kit of parts of claim 1, wherein the RNA of component B is a single stranded RNA.

4. The kit or kit of parts of claim 1, wherein the RNA of component B is a long chain RNA.

5. The kit or kit of parts of claim 1, wherein the syringe of component A is characterized by at least one of the following features
(ii) the syringe produces less than about 20% RNA agglomeration when incubated with an aqueous test formulation comprising RNA encapsulated in LNPs for 6 hours at 20° C.;
(iii) the syringe produces less than about 20% RNA agglomeration when incubated with the pharmaceutical composition of component B for 6 hours at 20° C.;
(iv) the syringe produces less than 10 mAU*min of extractable compounds in 20 μl of a 2-Propanol extract as determined by analytical (RP)HPLC; or
(v) a combination of any of (ii) to (iv).

6. The kit or kit of parts of claim 1, wherein the inner surface of the syringe barrel of component A is essentially free of silicone oils, the syringe plunger of component A is essentially free of silicone oils, the syringe plunger stopper of component A is essentially free of silicone oils, the needle adapter of component A is essentially free of silicone oils, and/or the needle hub of component A is essentially free of silicone oils.

7. The kit or kit of parts of claim 6, wherein the syringe barrel of component A comprises a polymer selected from olefin polymer, cyclic olefin copolymer (COP), polypropylene, polysterene, polyethylene, polycarbonate, or a combination of any of these.

8. The kit or kit of parts of claim 6, wherein the syringe barrel of component A comprises glass.

9. The kit or kit of parts of claim 8, wherein the syringe barrel of component A comprises a glass coating of the inner surface or a silicon dioxide coating of the inner surface.

10. The kit or kit of parts of claim 6, wherein the syringe plunger stopper of component A comprises a thermoplastic elastomer, a silicone polymer, or a rubber.

11. The kit or kit of parts of claim 6, wherein the syringe plunger stopper comprises a coating to reduce the gliding force needed for an injection.

12-20. (canceled)

21. The kit or kit of parts of claim 6, wherein the concentration of RNA in the pharmaceutical composition of component B is in a range of about 0.1 μg/ml to about 500 g/ml.

22. (canceled)

23. The kit or kit of parts of claim 21, wherein the RNA of the pharmaceutical composition of component B has an RNA integrity of at least about 70%.

24. (canceled)

25. The kit or kit of parts of claim 23, wherein the pharmaceutical composition of component B comprises less than about 20% free RNA.

26. The kit or kit of parts of claim 25, wherein the RNA of the pharmaceutical composition of component B is at least about 1000 nucleotides in length.

27-28. (canceled)

29. The kit or kit of parts of claim 26, wherein the RNA of the pharmaceutical composition comprises chemically modified nucleotides selected from pseudouridine (ψ) or N1-methylpseudouridine (ml).

30. The kit or kit of parts of claim 29, wherein the RNA of the pharmaceutical composition of component B is an mRNA.

31-69. (canceled)

70. A pre-filled syringe for injection containing a pharmaceutical composition comprising RNA, wherein the syringe used for obtaining the pre-filled syringe is characterized by that (i) the inner surface of the syringe barrel and/or the syringe plunger stopper is essentially free of silicone oils.

71-84. (canceled)

85. A method of treating or preventing a disorder or condition in a subject comprising the steps
(A) obtaining a pre-filled syringe as defined in claim 70; and
(B) injecting the pharmaceutical composition comprising RNA into the subject in need thereof using the syringe obtained in A) or B1).

86. (canceled)

87. A method for providing stable storage of a pharmaceutical composition or vaccine comprising RNA comprising:
a) obtaining a liquid composition or vaccine comprising RNA;
b) transferring the liquid composition or vaccine to a syringe, wherein the syringe is characterized by that (i) the inner surface of the syringe barrel and/or the syringe plunger stopper is essentially free of silicone oils;
c) obtaining a syringe containing a liquid composition or vaccine comprising RNA;
d) optionally, freezing of the obtained filled syringe or lyophilizing the composition contained in the obtained filled syringe;
e) stably storing the obtained filled syringe.

88-131. (canceled)


SEQUENCE LISTING (10 sequences)

SEQ ID NO: 1 - protein, 1273 aa, Artificial Sequence
  SARS-CoV-2 spike protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 1270 aa, Artificial Sequence
  SARS-CoV-2 spike protein
MFVFLVLLPLVSSQCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHISYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - RNA, 3822 nt, Artificial Sequence
  SARS-CoV-2 spike CDS
auguucguguuccugguccuccugccccuggugagcucccagugcgugaaccucaccacccgcacgcagcugccgcccgccuacaccaacagcuucacccggggcgucuacuaccccgacaagguguuccgcuccagcgugcugcacuccacccaggaccucuuccugcccuucuucagcaacgucacgugguuccacgccauccacguguccgggaccaacggcaccaagcgguucgacaacccggugcugcccuucaacgacggggucuacuucgcgagcaccgagaaguccaacaucauccgcggcuggaucuucgggacgacccucgacagcaagacccagucccugcugaucgugaacaacgccaccaacguggucaucaaggugugcgaguuccaguucugcaacgaccccuuccucggcguguacuaccacaagaacaacaagagcuggauggaguccgaguuccgggucuacagcuccgccaacaacugcacguucgaguacgugagccagcccuuccugauggaccuggagggcaagcaggggaacuucaagaaccuccgcgaguucguguucaagaacaucgacggcuacuucaagaucuacuccaagcacaccccgaucaaccugguccgggaccugccccagggguucagcgcccucgagccccugguggaccugcccaucggcaucaacaucacccgcuuccagacccuccuggcccugcaccgguccuaccucacgccgggggacagcagcuccggcuggaccgcgggcgccgccgccuacuacgugggguaccugcagccccgcaccuuccugcucaaguacaacgagaacggcaccaucacggacgcggucgacugcgcccuggacccccugagcgagaccaagugcacccucaaguccuucaccguggagaaggggaucuaccagacgagcaacuuccgggugcagcccaccgaguccaucguccgcuucccgaacaucaccaaccugugccccuucggcgagguguucaacgccacccgguucgccagcgucuacgccuggaaccgcaagcggaucuccaacugcguggcggacuacagcgugcuguacaacuccgccagcuucuccacguucaagugcuacggcgucagccccaccaagcucaacgaccugugcuucaccaacguguacgccgacuccuucgugauccgcggggacgagguccggcagaucgcccccggccagaccgggaagaucgccgacuacaacuacaagcugccggacgacuucacgggcugcgugaucgcguggaacagcaacaaccucgacuccaaggucgggggcaacuacaacuaccuguaccgccuguuccgcaagagcaaccucaagcccuucgagcgggacaucuccaccgagaucuaccaggccggcagcacccccugcaacgggguggagggcuucaacugcuacuucccccugcaguccuacggguuccagccgaccaacggcgugggguaccagcccuaccggguggucgugcugagcuucgagcuccugcacgcccccgccacggugugcggccccaagaaguccaccaaccuggucaagaacaagugcgugaacuucaacuucaacggccucaccgggaccggcgugcugaccgagagcaacaagaaguuccugccguuccagcaguucgggcgcgacaucgccgacacgaccgacgcgguccgcgacccccagacccuggagauccucgacaucacccccugcagcuucggcgggguguccgugaucacgccgggcaccaacaccagcaaccaggucgccgugcuguaccaggacgugaacugcaccgagguccccguggccauccacgccgaccagcugacgcccaccuggcggguguacuccaccggcagcaacgucuuccagacccgcgccgggugccucaucggcgcggagcacgugaacaacuccuacgagugcgacauccccaucggggccggcaucugcgccagcuaccagacgcagaccaacuccccgcggcgcgcccggagcguggcgucccagagcaucaucgccuacaccaugucccugggcgccgagaacagcgucgccuacuccaacaacagcaucgccauccccaccaacuucacgaucuccgugaccaccgagauccugcccgugagcaugaccaagacgagcgucgacugcaccauguacaucugcggggacuccaccgagugcagcaaccuccugcugcaguacggcuccuucugcacccagcucaaccgcgcgcugacggggaucgccguggagcaggacaagaacacccaggagguguucgcccaggucaagcagaucuacaagacgcccccgaucaaggacuucggcggguucaacuucagccagauccugcccgaccccuccaagcccagcaagcgguccuucaucgaggaccuccuguucaacaaggugacccuggccgacgccggcuucaucaagcaguacggcgacugccucggggacaucgcggcccgcgaccugaucugcgcccagaaguucaacggccugaccgugcucccgccccugcugaccgacgagaugaucgcccaguacaccagcgcccuccuggcggggaccaucacguccggcuggaccuucggggccggcgccgcccugcagauccccuucgcgaugcagauggccuaccgguucaacggcaucggggucacccagaacgugcucuacgagaaccagaagcugaucgccaaccaguucaacagcgccaucggcaagauccaggacucccugagcuccaccgccagcgcgcucgggaagcugcaggacguggucaaccagaacgcccaggcccugaacacgcucgugaagcagcuguccagcaacuucggcgccaucuccagcgugcugaacgacauccucucccgccuggacccgcccgaggccgaggugcagaucgaccggcugaucaccgggcgccuccagagccugcagaccuacgugacccagcagcugauccgggcggccgagauccgcgccagcgccaaccucgccgcgaccaagauguccgagugcguccugggccagagcaagcggguggacuucugcggcaagggguaccaccugauguccuucccccagagcgccccgcacggcguggucuuccuccacgugacguacguccccgcccaggagaagaacuucaccaccgcccccgcgaucugccacgacgggaaggcccacuucccccgcgagggcguguucguguccaacgggacccacugguucgucacgcagcggaacuucuacgagccgcagaucaucaccaccgacaacaccuucgugagcggcaacugcgacguggucaucggcaucgugaacaacacgguguacgacccccugcagcccgagcuggacuccuucaaggaggagcucgacaaguacuucaagaaccacaccagccccgacgucgaccugggggacaucuccggcaucaacgccagcguggugaacauccagaaggagaucgaccgccugaacgaggucgccaagaaccucaacgagucccugaucgaccugcaggagcucgggaaguacgagcaguacaucaaguggccgugguacaucuggcugggcuucaucgccggccugaucgcgaucgugauggugaccaucaugcucugcugcaugaccagcugcugcuccugccugaaggggugcugcagcugcggcuccugcugcaaguucgacgaggacgacagcgagcccguccugaagggggugaagcuccacuacacguga

SEQ ID NO: 4 - RNA, 3813 nt, Artificial Sequence
  SARS-CoV-2 spike CDS
auguucguguuccugguccuccugccccuggugagcucccagugcgugaacuucaccacccgcacgcagcugccgcccgccuacaccaacagcuucacccggggcgucuacuaccccgacaagguguuccgcuccagcgugcugcacuccacccaggaccucuuccugcccuucuucagcaacgucacgugguuccacgccauccacguguccgggaccaacggcaccaagcgguucgccaacccggugcugcccuucaacgacggggucuacuucgcgagcaccgagaaguccaacaucauccgcggcuggaucuucgggacgacccucgacagcaagacccagucccugcugaucgugaacaacgccaccaacguggucaucaaggugugcgaguuccaguucugcaacgaccccuuccucggcguguacuaccacaagaacaacaagagcuggauggaguccgaguuccgggucuacagcuccgccaacaacugcacguucgaguacgugagccagcccuuccugauggaccuggagggcaagcaggggaacuucaagaaccuccgcgaguucguguucaagaacaucgacggcuacuucaagaucuacuccaagcacaccccgaucaaccugguccggggccugccccagggguucagcgcccucgagccccugguggaccugcccaucggcaucaacaucacccgcuuccagacccuccacaucuccuaccucacgccgggggacagcagcuccggcuggaccgcgggcgccgccgccuacuacgugggguaccugcagccccgcaccuuccugcucaaguacaacgagaacggcaccaucacggacgcggucgacugcgcccuggacccccugagcgagaccaagugcacccucaaguccuucaccguggagaaggggaucuaccagacgagcaacuuccgggugcagcccaccgaguccaucguccgcuucccgaacaucaccaaccugugccccuucggcgagguguucaacgccacccgguucgccagcgucuacgccuggaaccgcaagcggaucuccaacugcguggcggacuacagcgugcuguacaacuccgccagcuucuccacguucaagugcuacggcgucagccccaccaagcucaacgaccugugcuucaccaacguguacgccgacuccuucgugauccgcggggacgagguccggcagaucgcccccggccagaccgggaacaucgccgacuacaacuacaagcugccggacgacuucacgggcugcgugaucgcguggaacagcaacaaccucgacuccaaggucgggggcaacuacaacuaccuguaccgccuguuccgcaagagcaaccucaagcccuucgagcgggacaucuccaccgagaucuaccaggccggcagcacccccugcaacggggugaagggcuucaacugcuacuucccccugcaguccuacggguuccagccgaccuacggcgugggguaccagcccuaccggguggucgugcugagcuucgagcuccugcacgcccccgccacggugugcggccccaagaaguccaccaaccuggucaagaacaagugcgugaacuucaacuucaacggccucaccgggaccggcgugcugaccgagagcaacaagaaguuccugccguuccagcaguucgggcgcgacaucgccgacacgaccgacgcgguccgcgacccccagacccuggagauccucgacaucacccccugcagcuucggcgggguguccgugaucacgccgggcaccaacaccagcaaccaggucgccgugcuguaccagggcgugaacugcaccgagguccccguggccauccacgccgaccagcugacgcccaccuggcggguguacuccaccggcagcaacgucuuccagacccgcgccgggugccucaucggcgcggagcacgugaacaacuccuacgagugcgacauccccaucggggccggcaucugcgccagcuaccagacgcagaccaacuccccgcggcgcgcccggagcguggcgucccagagcaucaucgccuacaccaugucccugggcguggagaacagcgucgccuacuccaacaacagcaucgccauccccaccaacuucacgaucuccgugaccaccgagauccugcccgugagcaugaccaagacgagcgucgacugcaccauguacaucugcggggacuccaccgagugcagcaaccuccugcugcaguacggcuccuucugcacccagcucaaccgcgcgcugacggggaucgccguggagcaggacaagaacacccaggagguguucgcccaggucaagcagaucuacaagacgcccccgaucaaggacuucggcggguucaacuucagccagauccugcccgaccccuccaagcccagcaagcgguccuucaucgaggaccuccuguucaacaaggugacccuggccgacgccggcuucaucaagcaguacggcgacugccucggggacaucgcggcccgcgaccugaucugcgcccagaaguucaacggccugaccgugcucccgccccugcugaccgacgagaugaucgcccaguacaccagcgcccuccuggcggggaccaucacguccggcuggaccuucggggccggcgccgcccugcagauccccuucgcgaugcagauggccuaccgguucaacggcaucggggucacccagaacgugcucuacgagaaccagaagcugaucgccaaccaguucaacagcgccaucggcaagauccaggacucccugagcuccaccgccagcgcgcucgggaagcugcaggacguggucaaccagaacgcccaggcccugaacacgcucgugaagcagcuguccagcaacuucggcgccaucuccagcgugcugaacgacauccucucccgccuggacccgcccgaggccgaggugcagaucgaccggcugaucaccgggcgccuccagagccugcagaccuacgugacccagcagcugauccgggcggccgagauccgcgccagcgccaaccucgccgcgaccaagauguccgagugcguccugggccagagcaagcggguggacuucugcggcaagggguaccaccugauguccuucccccagagcgccccgcacggcguggucuuccuccacgugacguacguccccgcccaggagaagaacuucaccaccgcccccgcgaucugccacgacgggaaggcccacuucccccgcgagggcguguucguguccaacgggacccacugguucgucacgcagcggaacuucuacgagccgcagaucaucaccaccgacaacaccuucgugagcggcaacugcgacguggucaucggcaucgugaacaacacgguguacgacccccugcagcccgagcuggacuccuucaaggaggagcucgacaaguacuucaagaaccacaccagccccgacgucgaccugggggacaucuccggcaucaacgccagcguggugaacauccagaaggagaucgaccgccugaacgaggucgccaagaaccucaacgagucccugaucgaccugcaggagcucgggaaguacgagcaguacaucaaguggccgugguacaucuggcugggcuucaucgccggccugaucgcgaucgugauggugaccaucaugcucugcugcaugaccagcugcugcuccugccugaaggggugcugcagcugcggcuccugcugcaaguucgacgaggacgacagcgagcccguccugaagggggugaagcuccacuacacguga

SEQ ID NO: 5 - RNA, 4039 nt, Artificial Sequence
  SARS-CoV-2 spike mRNA R9515
aggagaaagcuuaccauguucguguuccugguccuccugccccuggugagcucccagugcgugaaccucaccacccgcacgcagcugccgcccgccuacaccaacagcuucacccggggcgucuacuaccccgacaagguguuccgcuccagcgugcugcacuccacccaggaccucuuccugcccuucuucagcaacgucacgugguuccacgccauccacguguccgggaccaacggcaccaagcgguucgacaacccggugcugcccuucaacgacggggucuacuucgcgagcaccgagaaguccaacaucauccgcggcuggaucuucgggacgacccucgacagcaagacccagucccugcugaucgugaacaacgccaccaacguggucaucaaggugugcgaguuccaguucugcaacgaccccuuccucggcguguacuaccacaagaacaacaagagcuggauggaguccgaguuccgggucuacagcuccgccaacaacugcacguucgaguacgugagccagcccuuccugauggaccuggagggcaagcaggggaacuucaagaaccuccgcgaguucguguucaagaacaucgacggcuacuucaagaucuacuccaagcacaccccgaucaaccugguccgggaccugccccagggguucagcgcccucgagccccugguggaccugcccaucggcaucaacaucacccgcuuccagacccuccuggcccugcaccgguccuaccucacgccgggggacagcagcuccggcuggaccgcgggcgccgccgccuacuacgugggguaccugcagccccgcaccuuccugcucaaguacaacgagaacggcaccaucacggacgcggucgacugcgcccuggacccccugagcgagaccaagugcacccucaaguccuucaccguggagaaggggaucuaccagacgagcaacuuccgggugcagcccaccgaguccaucguccgcuucccgaacaucaccaaccugugccccuucggcgagguguucaacgccacccgguucgccagcgucuacgccuggaaccgcaagcggaucuccaacugcguggcggacuacagcgugcuguacaacuccgccagcuucuccacguucaagugcuacggcgucagccccaccaagcucaacgaccugugcuucaccaacguguacgccgacuccuucgugauccgcggggacgagguccggcagaucgcccccggccagaccgggaagaucgccgacuacaacuacaagcugccggacgacuucacgggcugcgugaucgcguggaacagcaacaaccucgacuccaaggucgggggcaacuacaacuaccuguaccgccuguuccgcaagagcaaccucaagcccuucgagcgggacaucuccaccgagaucuaccaggccggcagcacccccugcaacgggguggagggcuucaacugcuacuucccccugcaguccuacggguuccagccgaccaacggcgugggguaccagcccuaccggguggucgugcugagcuucgagcuccugcacgcccccgccacggugugcggccccaagaaguccaccaaccuggucaagaacaagugcgugaacuucaacuucaacggccucaccgggaccggcgugcugaccgagagcaacaagaaguuccugccguuccagcaguucgggcgcgacaucgccgacacgaccgacgcgguccgcgacccccagacccuggagauccucgacaucacccccugcagcuucggcgggguguccgugaucacgccgggcaccaacaccagcaaccaggucgccgugcuguaccaggacgugaacugcaccgagguccccguggccauccacgccgaccagcugacgcccaccuggcggguguacuccaccggcagcaacgucuuccagacccgcgccgggugccucaucggcgcggagcacgugaacaacuccuacgagugcgacauccccaucggggccggcaucugcgccagcuaccagacgcagaccaacuccccgcggcgcgcccggagcguggcgucccagagcaucaucgccuacaccaugucccugggcgccgagaacagcgucgccuacuccaacaacagcaucgccauccccaccaacuucacgaucuccgugaccaccgagauccugcccgugagcaugaccaagacgagcgucgacugcaccauguacaucugcggggacuccaccgagugcagcaaccuccugcugcaguacggcuccuucugcacccagcucaaccgcgcgcugacggggaucgccguggagcaggacaagaacacccaggagguguucgcccaggucaagcagaucuacaagacgcccccgaucaaggacuucggcggguucaacuucagccagauccugcccgaccccuccaagcccagcaagcgguccuucaucgaggaccuccuguucaacaaggugacccuggccgacgccggcuucaucaagcaguacggcgacugccucggggacaucgcggcccgcgaccugaucugcgcccagaaguucaacggccugaccgugcucccgccccugcugaccgacgagaugaucgcccaguacaccagcgcccuccuggcggggaccaucacguccggcuggaccuucggggccggcgccgcccugcagauccccuucgcgaugcagauggccuaccgguucaacggcaucggggucacccagaacgugcucuacgagaaccagaagcugaucgccaaccaguucaacagcgccaucggcaagauccaggacucccugagcuccaccgccagcgcgcucgggaagcugcaggacguggucaaccagaacgcccaggcccugaacacgcucgugaagcagcuguccagcaacuucggcgccaucuccagcgugcugaacgacauccucucccgccuggacccgcccgaggccgaggugcagaucgaccggcugaucaccgggcgccuccagagccugcagaccuacgugacccagcagcugauccgggcggccgagauccgcgccagcgccaaccucgccgcgaccaagauguccgagugcguccugggccagagcaagcggguggacuucugcggcaagggguaccaccugauguccuucccccagagcgccccgcacggcguggucuuccuccacgugacguacguccccgcccaggagaagaacuucaccaccgcccccgcgaucugccacgacgggaaggcccacuucccccgcgagggcguguucguguccaacgggacccacugguucgucacgcagcggaacuucuacgagccgcagaucaucaccaccgacaacaccuucgugagcggcaacugcgacguggucaucggcaucgugaacaacacgguguacgacccccugcagcccgagcuggacuccuucaaggaggagcucgacaaguacuucaagaaccacaccagccccgacgucgaccugggggacaucuccggcaucaacgccagcguggugaacauccagaaggagaucgaccgccugaacgaggucgccaagaaccucaacgagucccugaucgaccugcaggagcucgggaaguacgagcaguacaucaaguggccgugguacaucuggcugggcuucaucgccggccugaucgcgaucgugauggugaccaucaugcucugcugcaugaccagcugcugcuccugccugaaggggugcugcagcugcggcuccugcugcaaguucgacgaggacgacagcgagcccguccugaagggggugaagcuccacuacacgugaggacuaguuauaagacugacuagcccgaugggccucccaacgggcccuccuccccuccuugcaccgagauuaauaaaaaaaaaaaaaaaaaaaaaaaaaaaaaaaaaaaaaaaaaaaaaaaaaaaaaaaaaaaaaaaaugcaucccccccccccccccccccccccccccccccaaaggcucuuuucagagccaccagaauu

SEQ ID NO: 6 - RNA, 4030 nt, Artificial Sequence
  SARS-CoV-2 spike mRNA R10379
aggagaaagcuuaccauguucguguuccugguccuccugccccuggugagcucccagugcgugaacuucaccacccgcacgcagcugccgcccgccuacaccaacagcuucacccggggcgucuacuaccccgacaagguguuccgcuccagcgugcugcacuccacccaggaccucuuccugcccuucuucagcaacgucacgugguuccacgccauccacguguccgggaccaacggcaccaagcgguucgccaacccggugcugcccuucaacgacggggucuacuucgcgagcaccgagaaguccaacaucauccgcggcuggaucuucgggacgacccucgacagcaagacccagucccugcugaucgugaacaacgccaccaacguggucaucaaggugugcgaguuccaguucugcaacgaccccuuccucggcguguacuaccacaagaacaacaagagcuggauggaguccgaguuccgggucuacagcuccgccaacaacugcacguucgaguacgugagccagcccuuccugauggaccuggagggcaagcaggggaacuucaagaaccuccgcgaguucguguucaagaacaucgacggcuacuucaagaucuacuccaagcacaccccgaucaaccugguccggggccugccccagggguucagcgcccucgagccccugguggaccugcccaucggcaucaacaucacccgcuuccagacccuccacaucuccuaccucacgccgggggacagcagcuccggcuggaccgcgggcgccgccgccuacuacgugggguaccugcagccccgcaccuuccugcucaaguacaacgagaacggcaccaucacggacgcggucgacugcgcccuggacccccugagcgagaccaagugcacccucaaguccuucaccguggagaaggggaucuaccagacgagcaacuuccgggugcagcccaccgaguccaucguccgcuucccgaacaucaccaaccugugccccuucggcgagguguucaacgccacccgguucgccagcgucuacgccuggaaccgcaagcggaucuccaacugcguggcggacuacagcgugcuguacaacuccgccagcuucuccacguucaagugcuacggcgucagccccaccaagcucaacgaccugugcuucaccaacguguacgccgacuccuucgugauccgcggggacgagguccggcagaucgcccccggccagaccgggaacaucgccgacuacaacuacaagcugccggacgacuucacgggcugcgugaucgcguggaacagcaacaaccucgacuccaaggucgggggcaacuacaacuaccuguaccgccuguuccgcaagagcaaccucaagcccuucgagcgggacaucuccaccgagaucuaccaggccggcagcacccccugcaacggggugaagggcuucaacugcuacuucccccugcaguccuacggguuccagccgaccuacggcgugggguaccagcccuaccggguggucgugcugagcuucgagcuccugcacgcccccgccacggugugcggccccaagaaguccaccaaccuggucaagaacaagugcgugaacuucaacuucaacggccucaccgggaccggcgugcugaccgagagcaacaagaaguuccugccguuccagcaguucgggcgcgacaucgccgacacgaccgacgcgguccgcgacccccagacccuggagauccucgacaucacccccugcagcuucggcgggguguccgugaucacgccgggcaccaacaccagcaaccaggucgccgugcuguaccagggcgugaacugcaccgagguccccguggccauccacgccgaccagcugacgcccaccuggcggguguacuccaccggcagcaacgucuuccagacccgcgccgggugccucaucggcgcggagcacgugaacaacuccuacgagugcgacauccccaucggggccggcaucugcgccagcuaccagacgcagaccaacuccccgcggcgcgcccggagcguggcgucccagagcaucaucgccuacaccaugucccugggcguggagaacagcgucgccuacuccaacaacagcaucgccauccccaccaacuucacgaucuccgugaccaccgagauccugcccgugagcaugaccaagacgagcgucgacugcaccauguacaucugcggggacuccaccgagugcagcaaccuccugcugcaguacggcuccuucugcacccagcucaaccgcgcgcugacggggaucgccguggagcaggacaagaacacccaggagguguucgcccaggucaagcagaucuacaagacgcccccgaucaaggacuucggcggguucaacuucagccagauccugcccgaccccuccaagcccagcaagcgguccuucaucgaggaccuccuguucaacaaggugacccuggccgacgccggcuucaucaagcaguacggcgacugccucggggacaucgcggcccgcgaccugaucugcgcccagaaguucaacggccugaccgugcucccgccccugcugaccgacgagaugaucgcccaguacaccagcgcccuccuggcggggaccaucacguccggcuggaccuucggggccggcgccgcccugcagauccccuucgcgaugcagauggccuaccgguucaacggcaucggggucacccagaacgugcucuacgagaaccagaagcugaucgccaaccaguucaacagcgccaucggcaagauccaggacucccugagcuccaccgccagcgcgcucgggaagcugcaggacguggucaaccagaacgcccaggcccugaacacgcucgugaagcagcuguccagcaacuucggcgccaucuccagcgugcugaacgacauccucucccgccuggacccgcccgaggccgaggugcagaucgaccggcugaucaccgggcgccuccagagccugcagaccuacgugacccagcagcugauccgggcggccgagauccgcgccagcgccaaccucgccgcgaccaagauguccgagugcguccugggccagagcaagcggguggacuucugcggcaagggguaccaccugauguccuucccccagagcgccccgcacggcguggucuuccuccacgugacguacguccccgcccaggagaagaacuucaccaccgcccccgcgaucugccacgacgggaaggcccacuucccccgcgagggcguguucguguccaacgggacccacugguucgucacgcagcggaacuucuacgagccgcagaucaucaccaccgacaacaccuucgugagcggcaacugcgacguggucaucggcaucgugaacaacacgguguacgacccccugcagcccgagcuggacuccuucaaggaggagcucgacaaguacuucaagaaccacaccagccccgacgucgaccugggggacaucuccggcaucaacgccagcguggugaacauccagaaggagaucgaccgccugaacgaggucgccaagaaccucaacgagucccugaucgaccugcaggagcucgggaaguacgagcaguacaucaaguggccgugguacaucuggcugggcuucaucgccggccugaucgcgaucgugauggugaccaucaugcucugcugcaugaccagcugcugcuccugccugaaggggugcugcagcugcggcuccugcugcaaguucgacgaggacgacagcgagcccguccugaagggggugaagcuccacuacacgugaggacuaguuauaagacugacuagcccgaugggccucccaacgggcccuccuccccuccuugcaccgagauuaauaaaaaaaaaaaaaaaaaaaaaaaaaaaaaaaaaaaaaaaaaaaaaaaaaaaaaaaaaaaaaaaaugcaucccccccccccccccccccccccccccccccaaaggcucuuuucagagccaccagaauu

SEQ ID NO: 7 - RNA, 4094 nt, Artificial Sequence
  SARS-CoV-2 spike mRNA R9709
aggagagucccgcagucggcguccagcggcucugcuuguucgugugugugucguugcaggccuuauucaagcuuaccauguucguguuccugguccuccugccccuggugagcucccagugcgugaaccucaccacccgcacgcagcugccgcccgccuacaccaacagcuucacccggggcgucuacuaccccgacaagguguuccgcuccagcgugcugcacuccacccaggaccucuuccugcccuucuucagcaacgucacgugguuccacgccauccacguguccgggaccaacggcaccaagcgguucgacaacccggugcugcccuucaacgacggggucuacuucgcgagcaccgagaaguccaacaucauccgcggcuggaucuucgggacgacccucgacagcaagacccagucccugcugaucgugaacaacgccaccaacguggucaucaaggugugcgaguuccaguucugcaacgaccccuuccucggcguguacuaccacaagaacaacaagagcuggauggaguccgaguuccgggucuacagcuccgccaacaacugcacguucgaguacgugagccagcccuuccugauggaccuggagggcaagcaggggaacuucaagaaccuccgcgaguucguguucaagaacaucgacggcuacuucaagaucuacuccaagcacaccccgaucaaccugguccgggaccugccccagggguucagcgcccucgagccccugguggaccugcccaucggcaucaacaucacccgcuuccagacccuccuggcccugcaccgguccuaccucacgccgggggacagcagcuccggcuggaccgcgggcgccgccgccuacuacgugggguaccugcagccccgcaccuuccugcucaaguacaacgagaacggcaccaucacggacgcggucgacugcgcccuggacccccugagcgagaccaagugcacccucaaguccuucaccguggagaaggggaucuaccagacgagcaacuuccgggugcagcccaccgaguccaucguccgcuucccgaacaucaccaaccugugccccuucggcgagguguucaacgccacccgguucgccagcgucuacgccuggaaccgcaagcggaucuccaacugcguggcggacuacagcgugcuguacaacuccgccagcuucuccacguucaagugcuacggcgucagccccaccaagcucaacgaccugugcuucaccaacguguacgccgacuccuucgugauccgcggggacgagguccggcagaucgcccccggccagaccgggaagaucgccgacuacaacuacaagcugccggacgacuucacgggcugcgugaucgcguggaacagcaacaaccucgacuccaaggucgggggcaacuacaacuaccuguaccgccuguuccgcaagagcaaccucaagcccuucgagcgggacaucuccaccgagaucuaccaggccggcagcacccccugcaacgggguggagggcuucaacugcuacuucccccugcaguccuacggguuccagccgaccaacggcgugggguaccagcccuaccggguggucgugcugagcuucgagcuccugcacgcccccgccacggugugcggccccaagaaguccaccaaccuggucaagaacaagugcgugaacuucaacuucaacggccucaccgggaccggcgugcugaccgagagcaacaagaaguuccugccguuccagcaguucgggcgcgacaucgccgacacgaccgacgcgguccgcgacccccagacccuggagauccucgacaucacccccugcagcuucggcgggguguccgugaucacgccgggcaccaacaccagcaaccaggucgccgugcuguaccaggacgugaacugcaccgagguccccguggccauccacgccgaccagcugacgcccaccuggcggguguacuccaccggcagcaacgucuuccagacccgcgccgggugccucaucggcgcggagcacgugaacaacuccuacgagugcgacauccccaucggggccggcaucugcgccagcuaccagacgcagaccaacuccccgcggcgcgcccggagcguggcgucccagagcaucaucgccuacaccaugucccugggcgccgagaacagcgucgccuacuccaacaacagcaucgccauccccaccaacuucacgaucuccgugaccaccgagauccugcccgugagcaugaccaagacgagcgucgacugcaccauguacaucugcggggacuccaccgagugcagcaaccuccugcugcaguacggcuccuucugcacccagcucaaccgcgcgcugacggggaucgccguggagcaggacaagaacacccaggagguguucgcccaggucaagcagaucuacaagacgcccccgaucaaggacuucggcggguucaacuucagccagauccugcccgaccccuccaagcccagcaagcgguccuucaucgaggaccuccuguucaacaaggugacccuggccgacgccggcuucaucaagcaguacggcgacugccucggggacaucgcggcccgcgaccugaucugcgcccagaaguucaacggccugaccgugcucccgccccugcugaccgacgagaugaucgcccaguacaccagcgcccuccuggcggggaccaucacguccggcuggaccuucggggccggcgccgcccugcagauccccuucgcgaugcagauggccuaccgguucaacggcaucggggucacccagaacgugcucuacgagaaccagaagcugaucgccaaccaguucaacagcgccaucggcaagauccaggacucccugagcuccaccgccagcgcgcucgggaagcugcaggacguggucaaccagaacgcccaggcccugaacacgcucgugaagcagcuguccagcaacuucggcgccaucuccagcgugcugaacgacauccucucccgccuggacccgcccgaggccgaggugcagaucgaccggcugaucaccgggcgccuccagagccugcagaccuacgugacccagcagcugauccgggcggccgagauccgcgccagcgccaaccucgccgcgaccaagauguccgagugcguccugggccagagcaagcggguggacuucugcggcaagggguaccaccugauguccuucccccagagcgccccgcacggcguggucuuccuccacgugacguacguccccgcccaggagaagaacuucaccaccgcccccgcgaucugccacgacgggaaggcccacuucccccgcgagggcguguucguguccaacgggacccacugguucgucacgcagcggaacuucuacgagccgcagaucaucaccaccgacaacaccuucgugagcggcaacugcgacguggucaucggcaucgugaacaacacgguguacgacccccugcagcccgagcuggacuccuucaaggaggagcucgacaaguacuucaagaaccacaccagccccgacgucgaccugggggacaucuccggcaucaacgccagcguggugaacauccagaaggagaucgaccgccugaacgaggucgccaagaaccucaacgagucccugaucgaccugcaggagcucgggaaguacgagcaguacaucaaguggccgugguacaucuggcugggcuucaucgccggccugaucgcgaucgugauggugaccaucaugcucugcugcaugaccagcugcugcuccugccugaaggggugcugcagcugcggcuccugcugcaaguucgacgaggacgacagcgagcccguccugaagggggugaagcuccacuacacgugaggacuagucccuguucccagagcccacuuuuuuuucuuuuuuugaaauaaaauagccugucuuucagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 8 - RNA, 4085 nt, Artificial Sequence
  SARS-CoV-2 spike mRNA R10384
aggagagucccgcagucggcguccagcggcucugcuuguucgugugugugucguugcaggccuuauucaagcuuaccauguucguguuccugguccuccugccccuggugagcucccagugcgugaacuucaccacccgcacgcagcugccgcccgccuacaccaacagcuucacccggggcgucuacuaccccgacaagguguuccgcuccagcgugcugcacuccacccaggaccucuuccugcccuucuucagcaacgucacgugguuccacgccauccacguguccgggaccaacggcaccaagcgguucgccaacccggugcugcccuucaacgacggggucuacuucgcgagcaccgagaaguccaacaucauccgcggcuggaucuucgggacgacccucgacagcaagacccagucccugcugaucgugaacaacgccaccaacguggucaucaaggugugcgaguuccaguucugcaacgaccccuuccucggcguguacuaccacaagaacaacaagagcuggauggaguccgaguuccgggucuacagcuccgccaacaacugcacguucgaguacgugagccagcccuuccugauggaccuggagggcaagcaggggaacuucaagaaccuccgcgaguucguguucaagaacaucgacggcuacuucaagaucuacuccaagcacaccccgaucaaccugguccggggccugccccagggguucagcgcccucgagccccugguggaccugcccaucggcaucaacaucacccgcuuccagacccuccacaucuccuaccucacgccgggggacagcagcuccggcuggaccgcgggcgccgccgccuacuacgugggguaccugcagccccgcaccuuccugcucaaguacaacgagaacggcaccaucacggacgcggucgacugcgcccuggacccccugagcgagaccaagugcacccucaaguccuucaccguggagaaggggaucuaccagacgagcaacuuccgggugcagcccaccgaguccaucguccgcuucccgaacaucaccaaccugugccccuucggcgagguguucaacgccacccgguucgccagcgucuacgccuggaaccgcaagcggaucuccaacugcguggcggacuacagcgugcuguacaacuccgccagcuucuccacguucaagugcuacggcgucagccccaccaagcucaacgaccugugcuucaccaacguguacgccgacuccuucgugauccgcggggacgagguccggcagaucgcccccggccagaccgggaacaucgccgacuacaacuacaagcugccggacgacuucacgggcugcgugaucgcguggaacagcaacaaccucgacuccaaggucgggggcaacuacaacuaccuguaccgccuguuccgcaagagcaaccucaagcccuucgagcgggacaucuccaccgagaucuaccaggccggcagcacccccugcaacggggugaagggcuucaacugcuacuucccccugcaguccuacggguuccagccgaccuacggcgugggguaccagcccuaccggguggucgugcugagcuucgagcuccugcacgcccccgccacggugugcggccccaagaaguccaccaaccuggucaagaacaagugcgugaacuucaacuucaacggccucaccgggaccggcgugcugaccgagagcaacaagaaguuccugccguuccagcaguucgggcgcgacaucgccgacacgaccgacgcgguccgcgacccccagacccuggagauccucgacaucacccccugcagcuucggcgggguguccgugaucacgccgggcaccaacaccagcaaccaggucgccgugcuguaccagggcgugaacugcaccgagguccccguggccauccacgccgaccagcugacgcccaccuggcggguguacuccaccggcagcaacgucuuccagacccgcgccgggugccucaucggcgcggagcacgugaacaacuccuacgagugcgacauccccaucggggccggcaucugcgccagcuaccagacgcagaccaacuccccgcggcgcgcccggagcguggcgucccagagcaucaucgccuacaccaugucccugggcguggagaacagcgucgccuacuccaacaacagcaucgccauccccaccaacuucacgaucuccgugaccaccgagauccugcccgugagcaugaccaagacgagcgucgacugcaccauguacaucugcggggacuccaccgagugcagcaaccuccugcugcaguacggcuccuucugcacccagcucaaccgcgcgcugacggggaucgccguggagcaggacaagaacacccaggagguguucgcccaggucaagcagaucuacaagacgcccccgaucaaggacuucggcggguucaacuucagccagauccugcccgaccccuccaagcccagcaagcgguccuucaucgaggaccuccuguucaacaaggugacccuggccgacgccggcuucaucaagcaguacggcgacugccucggggacaucgcggcccgcgaccugaucugcgcccagaaguucaacggccugaccgugcucccgccccugcugaccgacgagaugaucgcccaguacaccagcgcccuccuggcggggaccaucacguccggcuggaccuucggggccggcgccgcccugcagauccccuucgcgaugcagauggccuaccgguucaacggcaucggggucacccagaacgugcucuacgagaaccagaagcugaucgccaaccaguucaacagcgccaucggcaagauccaggacucccugagcuccaccgccagcgcgcucgggaagcugcaggacguggucaaccagaacgcccaggcccugaacacgcucgugaagcagcuguccagcaacuucggcgccaucuccagcgugcugaacgacauccucucccgccuggacccgcccgaggccgaggugcagaucgaccggcugaucaccgggcgccuccagagccugcagaccuacgugacccagcagcugauccgggcggccgagauccgcgccagcgccaaccucgccgcgaccaagauguccgagugcguccugggccagagcaagcggguggacuucugcggcaagggguaccaccugauguccuucccccagagcgccccgcacggcguggucuuccuccacgugacguacguccccgcccaggagaagaacuucaccaccgcccccgcgaucugccacgacgggaaggcccacuucccccgcgagggcguguucguguccaacgggacccacugguucgucacgcagcggaacuucuacgagccgcagaucaucaccaccgacaacaccuucgugagcggcaacugcgacguggucaucggcaucgugaacaacacgguguacgacccccugcagcccgagcuggacuccuucaaggaggagcucgacaaguacuucaagaaccacaccagccccgacgucgaccugggggacaucuccggcaucaacgccagcguggugaacauccagaaggagaucgaccgccugaacgaggucgccaagaaccucaacgagucccugaucgaccugcaggagcucgggaaguacgagcaguacaucaaguggccgugguacaucuggcugggcuucaucgccggccugaucgcgaucgugauggugaccaucaugcucugcugcaugaccagcugcugcuccugccugaaggggugcugcagcugcggcuccugcugcaaguucgacgaggacgacagcgagcccguccugaagggggugaagcuccacuacacgugaggacuagucccuguucccagagcccacuuuuuuuucuuuuuuugaaauaaaauagccugucuuucagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 9 - RNA, 4070 nt, Artificial Sequence
  SARS-CoV-2 spike mRNA R10153
aggagagucccgcagucggcguccagcggcucugcuuguucgugugugugucguugcaggccuuauucaagcuuaccauguucguguuccugguccuccugccccuggugagcucccagugcgugaaccucaccacccgcacgcagcugccgcccgccuacaccaacagcuucacccggggcgucuacuaccccgacaagguguuccgcuccagcgugcugcacuccacccaggaccucuuccugcccuucuucagcaacgucacgugguuccacgccauccacguguccgggaccaacggcaccaagcgguucgacaacccggugcugcccuucaacgacggggucuacuucgcgagcaccgagaaguccaacaucauccgcggcuggaucuucgggacgacccucgacagcaagacccagucccugcugaucgugaacaacgccaccaacguggucaucaaggugugcgaguuccaguucugcaacgaccccuuccucggcguguacuaccacaagaacaacaagagcuggauggaguccgaguuccgggucuacagcuccgccaacaacugcacguucgaguacgugagccagcccuuccugauggaccuggagggcaagcaggggaacuucaagaaccuccgcgaguucguguucaagaacaucgacggcuacuucaagaucuacuccaagcacaccccgaucaaccugguccgggaccugccccagggguucagcgcccucgagccccugguggaccugcccaucggcaucaacaucacccgcuuccagacccuccuggcccugcaccgguccuaccucacgccgggggacagcagcuccggcuggaccgcgggcgccgccgccuacuacgugggguaccugcagccccgcaccuuccugcucaaguacaacgagaacggcaccaucacggacgcggucgacugcgcccuggacccccugagcgagaccaagugcacccucaaguccuucaccguggagaaggggaucuaccagacgagcaacuuccgggugcagcccaccgaguccaucguccgcuucccgaacaucaccaaccugugccccuucggcgagguguucaacgccacccgguucgccagcgucuacgccuggaaccgcaagcggaucuccaacugcguggcggacuacagcgugcuguacaacuccgccagcuucuccacguucaagugcuacggcgucagccccaccaagcucaacgaccugugcuucaccaacguguacgccgacuccuucgugauccgcggggacgagguccggcagaucgcccccggccagaccgggaagaucgccgacuacaacuacaagcugccggacgacuucacgggcugcgugaucgcguggaacagcaacaaccucgacuccaaggucgggggcaacuacaacuaccuguaccgccuguuccgcaagagcaaccucaagcccuucgagcgggacaucuccaccgagaucuaccaggccggcagcacccccugcaacgggguggagggcuucaacugcuacuucccccugcaguccuacggguuccagccgaccaacggcgugggguaccagcccuaccggguggucgugcugagcuucgagcuccugcacgcccccgccacggugugcggccccaagaaguccaccaaccuggucaagaacaagugcgugaacuucaacuucaacggccucaccgggaccggcgugcugaccgagagcaacaagaaguuccugccguuccagcaguucgggcgcgacaucgccgacacgaccgacgcgguccgcgacccccagacccuggagauccucgacaucacccccugcagcuucggcgggguguccgugaucacgccgggcaccaacaccagcaaccaggucgccgugcuguaccaggacgugaacugcaccgagguccccguggccauccacgccgaccagcugacgcccaccuggcggguguacuccaccggcagcaacgucuuccagacccgcgccgggugccucaucggcgcggagcacgugaacaacuccuacgagugcgacauccccaucggggccggcaucugcgccagcuaccagacgcagaccaacuccccgcggcgcgcccggagcguggcgucccagagcaucaucgccuacaccaugucccugggcgccgagaacagcgucgccuacuccaacaacagcaucgccauccccaccaacuucacgaucuccgugaccaccgagauccugcccgugagcaugaccaagacgagcgucgacugcaccauguacaucugcggggacuccaccgagugcagcaaccuccugcugcaguacggcuccuucugcacccagcucaaccgcgcgcugacggggaucgccguggagcaggacaagaacacccaggagguguucgcccaggucaagcagaucuacaagacgcccccgaucaaggacuucggcggguucaacuucagccagauccugcccgaccccuccaagcccagcaagcgguccuucaucgaggaccuccuguucaacaaggugacccuggccgacgccggcuucaucaagcaguacggcgacugccucggggacaucgcggcccgcgaccugaucugcgcccagaaguucaacggccugaccgugcucccgccccugcugaccgacgagaugaucgcccaguacaccagcgcccuccuggcggggaccaucacguccggcuggaccuucggggccggcgccgcccugcagauccccuucgcgaugcagauggccuaccgguucaacggcaucggggucacccagaacgugcucuacgagaaccagaagcugaucgccaaccaguucaacagcgccaucggcaagauccaggacucccugagcuccaccgccagcgcgcucgggaagcugcaggacguggucaaccagaacgcccaggcccugaacacgcucgugaagcagcuguccagcaacuucggcgccaucuccagcgugcugaacgacauccucucccgccuggacccgcccgaggccgaggugcagaucgaccggcugaucaccgggcgccuccagagccugcagaccuacgugacccagcagcugauccgggcggccgagauccgcgccagcgccaaccucgccgcgaccaagauguccgagugcguccugggccagagcaagcggguggacuucugcggcaagggguaccaccugauguccuucccccagagcgccccgcacggcguggucuuccuccacgugacguacguccccgcccaggagaagaacuucaccaccgcccccgcgaucugccacgacgggaaggcccacuucccccgcgagggcguguucguguccaacgggacccacugguucgucacgcagcggaacuucuacgagccgcagaucaucaccaccgacaacaccuucgugagcggcaacugcgacguggucaucggcaucgugaacaacacgguguacgacccccugcagcccgagcuggacuccuucaaggaggagcucgacaaguacuucaagaaccacaccagccccgacgucgaccugggggacaucuccggcaucaacgccagcguggugaacauccagaaggagaucgaccgccugaacgaggucgccaagaaccucaacgagucccugaucgaccugcaggagcucgggaaguacgagcaguacaucaaguggccgugguacaucuggcugggcuucaucgccggccugaucgcgaucgugauggugaccaucaugcucugcugcaugaccagcugcugcuccugccugaaggggugcugcagcugcggcuccugcugcaaguucgacgaggacgacagcgagcccguccugaagggggugaagcuccacuacacgugaggacuagucccuguucccagagcccacuuuuuuuucuuuuuuugaaauaaaauagccugucuuucagaucuaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 10 - RNA, 4061 nt, Artificial Sequence
  SARS-CoV-2 spike mRNA R10452
aggagagucccgcagucggcguccagcggcucugcuuguucgugugugugucguugcaggccuuauucaagcuuaccauguucguguuccugguccuccugccccuggugagcucccagugcgugaacuucaccacccgcacgcagcugccgcccgccuacaccaacagcuucacccggggcgucuacuaccccgacaagguguuccgcuccagcgugcugcacuccacccaggaccucuuccugcccuucuucagcaacgucacgugguuccacgccauccacguguccgggaccaacggcaccaagcgguucgccaacccggugcugcccuucaacgacggggucuacuucgcgagcaccgagaaguccaacaucauccgcggcuggaucuucgggacgacccucgacagcaagacccagucccugcugaucgugaacaacgccaccaacguggucaucaaggugugcgaguuccaguucugcaacgaccccuuccucggcguguacuaccacaagaacaacaagagcuggauggaguccgaguuccgggucuacagcuccgccaacaacugcacguucgaguacgugagccagcccuuccugauggaccuggagggcaagcaggggaacuucaagaaccuccgcgaguucguguucaagaacaucgacggcuacuucaagaucuacuccaagcacaccccgaucaaccugguccggggccugccccagggguucagcgcccucgagccccugguggaccugcccaucggcaucaacaucacccgcuuccagacccuccacaucuccuaccucacgccgggggacagcagcuccggcuggaccgcgggcgccgccgccuacuacgugggguaccugcagccccgcaccuuccugcucaaguacaacgagaacggcaccaucacggacgcggucgacugcgcccuggacccccugagcgagaccaagugcacccucaaguccuucaccguggagaaggggaucuaccagacgagcaacuuccgggugcagcccaccgaguccaucguccgcuucccgaacaucaccaaccugugccccuucggcgagguguucaacgccacccgguucgccagcgucuacgccuggaaccgcaagcggaucuccaacugcguggcggacuacagcgugcuguacaacuccgccagcuucuccacguucaagugcuacggcgucagccccaccaagcucaacgaccugugcuucaccaacguguacgccgacuccuucgugauccgcggggacgagguccggcagaucgcccccggccagaccgggaacaucgccgacuacaacuacaagcugccggacgacuucacgggcugcgugaucgcguggaacagcaacaaccucgacuccaaggucgggggcaacuacaacuaccuguaccgccuguuccgcaagagcaaccucaagcccuucgagcgggacaucuccaccgagaucuaccaggccggcagcacccccugcaacggggugaagggcuucaacugcuacuucccccugcaguccuacggguuccagccgaccuacggcgugggguaccagcccuaccggguggucgugcugagcuucgagcuccugcacgcccccgccacggugugcggccccaagaaguccaccaaccuggucaagaacaagugcgugaacuucaacuucaacggccucaccgggaccggcgugcugaccgagagcaacaagaaguuccugccguuccagcaguucgggcgcgacaucgccgacacgaccgacgcgguccgcgacccccagacccuggagauccucgacaucacccccugcagcuucggcgggguguccgugaucacgccgggcaccaacaccagcaaccaggucgccgugcuguaccagggcgugaacugcaccgagguccccguggccauccacgccgaccagcugacgcccaccuggcggguguacuccaccggcagcaacgucuuccagacccgcgccgggugccucaucggcgcggagcacgugaacaacuccuacgagugcgacauccccaucggggccggcaucugcgccagcuaccagacgcagaccaacuccccgcggcgcgcccggagcguggcgucccagagcaucaucgccuacaccaugucccugggcguggagaacagcgucgccuacuccaacaacagcaucgccauccccaccaacuucacgaucuccgugaccaccgagauccugcccgugagcaugaccaagacgagcgucgacugcaccauguacaucugcggggacuccaccgagugcagcaaccuccugcugcaguacggcuccuucugcacccagcucaaccgcgcgcugacggggaucgccguggagcaggacaagaacacccaggagguguucgcccaggucaagcagaucuacaagacgcccccgaucaaggacuucggcggguucaacuucagccagauccugcccgaccccuccaagcccagcaagcgguccuucaucgaggaccuccuguucaacaaggugacccuggccgacgccggcuucaucaagcaguacggcgacugccucggggacaucgcggcccgcgaccugaucugcgcccagaaguucaacggccugaccgugcucccgccccugcugaccgacgagaugaucgcccaguacaccagcgcccuccuggcggggaccaucacguccggcuggaccuucggggccggcgccgcccugcagauccccuucgcgaugcagauggccuaccgguucaacggcaucggggucacccagaacgugcucuacgagaaccagaagcugaucgccaaccaguucaacagcgccaucggcaagauccaggacucccugagcuccaccgccagcgcgcucgggaagcugcaggacguggucaaccagaacgcccaggcccugaacacgcucgugaagcagcuguccagcaacuucggcgccaucuccagcgugcugaacgacauccucucccgccuggacccgcccgaggccgaggugcagaucgaccggcugaucaccgggcgccuccagagccugcagaccuacgugacccagcagcugauccgggcggccgagauccgcgccagcgccaaccucgccgcgaccaagauguccgagugcguccugggccagagcaagcggguggacuucugcggcaagggguaccaccugauguccuucccccagagcgccccgcacggcguggucuuccuccacgugacguacguccccgcccaggagaagaacuucaccaccgcccccgcgaucugccacgacgggaaggcccacuucccccgcgagggcguguucguguccaacgggacccacugguucgucacgcagcggaacuucuacgagccgcagaucaucaccaccgacaacaccuucgugagcggcaacugcgacguggucaucggcaucgugaacaacacgguguacgacccccugcagcccgagcuggacuccuucaaggaggagcucgacaaguacuucaagaaccacaccagccccgacgucgaccugggggacaucuccggcaucaacgccagcguggugaacauccagaaggagaucgaccgccugaacgaggucgccaagaaccucaacgagucccugaucgaccugcaggagcucgggaaguacgagcaguacaucaaguggccgugguacaucuggcugggcuucaucgccggccugaucgcgaucgugauggugaccaucaugcucugcugcaugaccagcugcugcuccugccugaaggggugcugcagcugcggcuccugcugcaaguucgacgaggacgacagcgagcccguccugaagggggugaagcuccacuacacgugaggacuagucccuguucccagagcccacuuuuuuuucuuuuuuugaaauaaaauagccugucuuucagaucuaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa